(19) United States
(12) Patent Application Publication
(10) Pub. No.: US 2011/0038888 A1
(43) Pub. Date: Feb. 17, 2011

(54) ADJUVANT COMPOSITIONS COMPRISING POLY-IC AND A CATIONIC POLYMER

(76) Inventor: Peter Emtage, San Diego, CA (US)

Correspondence Address: WILMERHALE/NEW YORK, 399 PARK AVENUE, NEW YORK, NY, 10022, US

(21) Appl. No.: 12/812,544

(22) PCT Filed: Jan. 9, 2009
(86) PCT No.: PCT/CA2009/000035

§ 371 Date: Oct. 29, 2010

Related U.S. Application Data
(60) Provisional application No. 61/050,097, filed on May 2, 2008.
(60) Provisional application No. 61/020,320, filed on Jan. 10, 2008.
(60) Provisional application No. 61/085,673, filed on Aug. 1, 2008.
(60) Provisional application No. 61/119,344, filed on Dec. 2, 2008.

(51) Int. Cl. A61K 39/00; A61K 47/30; A61K 39/02; A61K 39/12; C12N 5/0784; C12N 5/078; A61P 35/00; A61P 37/04

(52) U.S. Cl. 424/192.1; 424/280.1; 424/184.1; 424/234.1; 424/204.1; 424/274.1; 424/277.1; 435/377; 435/375

(57) ABSTRACT

An adjuvant composition comprising polyIC and a cationic polymer; the cationic polymer comprising from about 100 to about 500 amino acid residues, wherein the mass:mass ratio of the polyIC:cationic polymer is from about 4:1.5 to about 4:3 is described.

### FIELD OF INVENTION

The present invention relates to the field of immunology, and immunostimulatory agents. More specifically, the present invention provides compositions comprising polycationic polymers and immunostimulatory nucleic acids.

### BACKGROUND OF THE INVENTION

The innate immune system has a role as both a ‘first-line’ of defense for an invading pathogen, and also a supporting role for the adaptive immune response. Toll-like receptors (TLRs) are one family of receptors that have a key role in the initiation of both the innate and adaptive immune response. TLRs respond individually to various infectious agent hallmarks, for example, TLR4 is particularly responsive to lipopolysaccharides, TLR9 preferentially responds to methylated nucleic acids, while dsRNAs are the preferred agonist of TLR3.

Double-stranded RNA (dsRNA) is a common replicative intermediate of viral infections. TLR3 initiates a non-specific innate immune response when viral replication occurs in the host, or when a host is exposed to viral replication mimics such as polyIC double-stranded RNA. Stimulation of TLR3 leads to activation of NF-kB and subsequent production of inflammatory cytokines including interferons, which in turn enhance the adaptive immune response by stimulating increased expression of MHC class I and class II.

The immunostimulatory characteristic of dsRNAs has been of interest with respect to the development of cancer therapeutics. The use of polyIC as an adjuvant and used in combination with therapeutic agents is well known. Furthermore, PolyIC dsRNA has been combined with other agents to improve stability. U.S. Pat. No. 4,346,538 describes polyIC complexes comprising relatively high molecular weight polyIC, poly-L-lysine in a MW range of 13-35 kDa and carboxymethylcellulose (“polyICLC”); and methods of preparation and using such compositions. The use of polyICLC as a therapeutic agent for the treatment of some cancers, some viral diseases such as HIV or Ebola, and also in multiple sclerosis has also been suggested (US Publication 2006/0223742).

Other dsRNAs have also been demonstrated to have some potential as cancer therapeutic agents. For example, dsRNAs in combination with lymphokines have been described as having a synergistic effect as therapeutic agents for treatment of melanoma (EP 0281380). TLR3 agonists, including polyIC and polyAU, for use in improved methods in treating cancers have also been described (US 2006/0110746).

Zhu et al (J. Translational Medicine 2007 5:10 doi:10.1186/1479-5876-5-10) describes a combination of polyICLC (administered intramuscularly) and specific tumor immunogens (administered subcutaneously in combination with IFA) as an effective treatment for mice bearing CNS gliomas. U.S. Pat. No. 3,952,097 to Levy describes polyICLC compositions and methods of making them. US 2004/0005998 and US 2006/0223742, both to Salazar, describe methods for preparation and uses of compositions comprising polyICLC.

Some PolyI:C compositions have been used in treatment of chronic fatigue syndrome, and in combination with antiviral agents in treatment of HIV infection variants (Thompson et al., Eur J Clin Microbiol Infect Dis. 1996 July; 15(7):580-7; Gillespie et al., In Vivo. 1994 May-June; 8(3):375-81; Strayer et al., Clin Infect Dis 1994 January; 18 Suppl 1:S88-95).

PolyIC is an RNA molecule and thus subject to degradation by the immune responses it stimulates, as a part of the innate viral defense response. dsRNA adjuvants with improved stability, suitable for co-administration as an adjuvant with at least one therapeutic agent, for example but not limited to a viral immunogen, and capable of enhancing the immunostimulatory activity of the viral immunogen are desired.

Other specific oligonucleotide motifs have been identified as having immunostimulatory effects, for example CpG dinucleotides. Some unmethylated CpG motifs in DNA are TLR9 agonists, and have been proposed as cancer therapeutics (Krieg A M. 2007 J. Clin Invest 117:1184-94). U.S. Pat. No. 7,148,191 describes an antigenic composition comprising, a polycationic peptide and a nucleic acid comprising inosine and cytosine, for use in combination with a small (6-20 amino acids) antigen. WO 01/93905 describes immunostimulatory oligodeoxynucleotides that exclude CpG motifs, citing side effects such as high systemic TNF-alpha and a lack of specificity.

Therapeutic nucleic acids, including RNAs, may be subject to degradation by the immune response that they stimulate, as part of the innate viral defense response. U.S. Pat. No. 6,194,388 (and references therein) teach that exchanging deoxyribose nucleosides for ribose nucleosides in the nucleic acid compositions is not effective in increasing stability, as the specific form the ribose sugar appears to be required for immune activation. Increasing the dose does not circumvent the stability issues either, as toxicity is dose-dependent.

Adjuvants with improved stability, suitable for co-administration in combination with at least one therapeutic agent, for example, but not limited to, a viral immunogen, and capable of enhancing the immunostimulatory activity of the viral immunogen are desired.

U.S. Pat. No. 7,105,162 to Schmidt describes a pharmaceutical composition comprising an antigenic peptide that fits into the binding form of an MHC molecule in combination with a cationic polypeptide, such as polyarginine with a degree of polymerization from about 15 to about 500.

U.S. Pat. No. 7,148,191 to Egyed describes a pharmaceutical composition comprising a 6-20 amino acid antigen (recognized by T-cells), a polycationic peptide and a nucleic acid based on inosine and cytosine. The particular polycationic peptide used by Egyed in the experimental examples was a polyarginine product obtained from Sigma, with a degree of polymerization of 60. Egyed teaches an example of a short polyR product with a dp of 60 (about 10,000 Da), and used in a mass:mass ratio with polyIC in a range of 0.5:1 to 3:1 (polyIC:polyarginine).

U.S. Pat. No. 6,869,607 to Buschle describes vaccine formulations, comprising peptides and agents for making the vaccine isotonic. Sorbitol in combination with polyarginine is exemplified as such a vaccine formulation.

CA 1,326,999 to Carter describes anti-inflammatory disease compositions comprising a dsRNA and a carrier; the compositions may further comprise carboxymethylcellulose or lysine.

U.S. Pat. Nos. 3,679,654 and 3,725,545 to Maes describes products capable of enhancing antibody production in response to an antigen. Polymers of basic amino acids are discussed, including maximizing an antibody response to an antigen complexed with polyIC and protamine sulfate. Polylysine is described as the least efficacious amino acid polymer. (column 6, lines 40-49)

### SUMMARY OF THE INVENTION

The present invention relates to immunology, and immunostimulatory agents. More specifically, the present invention provides compositions comprising polycationic polymers and immunostimulatory nucleic acids. The invention provides improved adjuvant compositions comprising PolyIC and polyarginine, or polylysine.

It is an object of the invention to provide an improved adjuvant compound comprising an immunostimulatory nucleic acid and a polycationic polymer.

In accordance with one aspect of the invention, there is provided an adjuvant composition comprising polyIC and a cationic polymer; the cationic polymer comprising from about 100 to about 700 amino acid residues, wherein the mass:mass ratio of the polyIC:cationic polymer is from about 4:1.4 to about 4:3. The polyIC may have an average molecular mass from about 100 to about 5000 kDa. The polycationic polymer may be polyarginine, polylysine, or polyornithine.

In accordance with another aspect of the invention, there is provided a method of inducing dendritic cell maturation comprising administering to a subject a composition comprising polyIC and a cationic polymer; the cationic polymer comprising from about 100 to about 700 amino acid residues, wherein the mass:mass ratio of the polyIC:cationinc polymer is from about 4:1.4 to about 4:3. The polyIC may have an average molecular mass from about 100 to about 5000 kDa. The polycationic polymer may be polyarginine, polylysine, or polyornithine. The composition may also comprise an immunogen present at an amount from about 0.1 ug to about 20 mg. The immunogen may comprise a bacterial immunogen, a viral immunogen, a fungal immunogen, a killed whole-organism, a protein, a peptide, a fusion protein, a fusion peptide, a recombinant protein, a recombinant peptide, one or more than one fragment or portion of a killed whole-organism, a tumor antigen, a tumor-derived antigen, an antigen found in association with a cancer, a heat shock protein, a heat shock fusion protein and HspE7. The composition may be administered subcutaneously, intravenously, intraperitoneally, intramuscularly, intranasally, or topically.

In accordance with another aspect of the invention, there is provided a method of decreasing the size or number, or size and number, of a tumor or tumors, or tumor cells in a subject, comprising administering to a subject a composition comprising polyIC and a cationic polymer; the cationic polymer comprising from about 100 to about 700 amino acid residues, wherein the mass:mass ratio of the polyIC:cationic polymer is from about 4:1.4 to about 4:3. The polyIC may have an average molecular mass from about 100 to about 5000 kDa. The polycationic polymer may be polyarginine, polylysine, or polyornithine. The composition may also comprise an immunogen present at an amount from about 0.1 ug to about 20 mg. The immunogen may comprise a bacterial immunogen, a viral immunogen, a fungal immunogen, a killed whole-organism, a protein, a peptide, a fusion protein, a fusion peptide, a recombinant protein, a recombinant peptide, one or more than one fragment or portion of a killed whole-organism, a tumor antigen, a tumor-derived antigen, an antigen found in association with a cancer, a heat shock protein, a heat shock fusion protein and HspE7. The composition may be administered subcutaneously, intravenously, intraperitoneally, intramuscularly, intranasally, or topically.

In accordance with another aspect of the invention, there is provided a use of a composition comprising polyIC and a cationic polymer for co-stimulation of the TLR3 and TLR8 receptors. The composition may also comprise an immunogen present at an amount from about 0.1 ug to about 20 mg. The immunogen may comprise a bacterial immunogen, a viral immunogen, a fungal immunogen, a killed whole-organism, a protein, a peptide, a fusion protein, a fusion peptide, a recombinant protein, a recombinant peptide, one or more than one fragment or portion of a killed whole-organism, a tumor antigen, a tumor-derived antigen, an antigen found in association with a cancer, a heat shock protein, a heat shock fusion protein and HspE7. The composition may be administered subcutaneously, intravenously, intraperitoneally, intramuscularly, intranasally, or topically. The cationic polymer comprises from about 100 to about 500 amino acid residues, wherein the mass:mass ratio of the polyIC:cationic polymer is from about 4:1.5 to about 4:3. The polycationic polymer may be polyarginine, polylysine, or polyornithine. The polyIC may have an average molecular mass from about 100 to about 5000 kDa.

In accordance with another aspect of the invention, there is provided a TLR8 agonist comprising polyIC and a cationic polymer; the cationic polymer comprising from about 100 to about 500 amino acid residues, wherein the mass:mass ratio of the polyIC:cationic polymer is from about 4:1.5 to about 4:3. The polycationic polymer may be polyarginine, polylysine, or polyornithine. The polyIC may have an average molecular mass from about 100 to about 5000 kDa.

The present invention also pertains to a method of inducing dendritic cell maturation comprising administering a composition to a subject comprising a TLR3 agonist and a TLR8 agonist, thereby inducing dendritic cell maturation. The TLR3 agonist may be polyIC, and the TLR8 agonist may be a combination of polyIC and a cationic polymer comprising from about 100 to about 700 amino acid residues, the mass:mass ratio of the polyIC:cationic polymer is from about 4:1.5 to about 4:3. The polycationic polymer may be polyarginine, polylysine, or polyornithine. The polyIC may have an average molecular mass from about 100 to about 5000 kDa. The composition may also comprise an immunogen present at an amount from about 0.1 ug to about 20 mg. The immunogen may comprise a bacterial immunogen, a viral immunogen, a fungal immunogen, a killed whole-organism, a protein, a peptide, a fusion protein, a fusion peptide, a recombinant protein, a recombinant peptide, one or more than one fragment or portion of a killed whole-organism, a tumor antigen, a tumor-derived antigen, an antigen found in association with a cancer, a heat shock protein, a heat shock fusion protein and HspE7. The composition may be administered subcutaneously, intravenously, intraperitoneally, intramuscularly, intranasally, or topically.

In accordance with another aspect of the invention, there is provided a method of increasing the biological activity of an immunogen in a subject, comprising administering the immunogen in combination with an immune stimulant comprising a TLR3 and a TLR8 agonist to the subject, thereby increasing the biological activity of the immunogen. The TLR3 agonist may be polyIC, and the TLR8 agonist may be a combination of polyIC and a cationic polymer comprising from about 100 to about 700 amino acid residues, the mass:mass ratio of the polyIC:cationic polymer is from about 4:1.5 to about 4:3. The polycationic polymer may be polyarginine, polylysine, or polyornithine. The polyIC may have an average molecular mass from about 100 to about 5000 kDa. The composition may also comprise an immunogen present at an amount from about 0.1 ug to about 20 mg. The immunogen may comprise a bacterial immunogen, a viral immunogen, a fungal immunogen, a killed whole-organism, a protein, a peptide, a fusion protein, a fusion peptide, a recombinant protein, a recombinant peptide, one or more than one fragment or portion of a killed whole-organism, a tumor antigen, a tumor-derived antigen, an antigen found in association with a cancer, a heat shock protein, a heat shock fusion protein and HspE7. The composition may be administered subcutaneously, intravenously, intraperitoneally, intramuscularly, intranasally, or topically.

In accordance with another aspect of the invention, there is provided a method of inducing an immune cell comprising, administering a composition comprising polyIC and a cationic polymer; the cationic polymer comprising from about 100 to about 700 amino acid residues; and the mass:mass ratio of the polyIC:cationic polymer is from about 4:1.5 to about 4:3. The composition may also comprise an immunogen present at an amount from about 0.1 ug to about 20 mg. The immunogen may comprise a bacterial immunogen, a viral immunogen, a fungal immunogen, a killed whole-organism, a protein, a peptide, a fusion protein, a fusion peptide, a recombinant protein, a recombinant peptide, one or more than one fragment or portion of a killed whole-organism, a tumor antigen, a tumor-derived antigen, an antigen found in association with a cancer, a heat shock protein, a heat shock fusion protein and HspE7. The polyIC may have an average molecular mass from about 100 to about 5000 kDa. The polycationic polymer may be polyarginine, polylysine, or polyornithine.

In accordance with another aspect of the invention, there is provided a method of inducing cytokine production in a cell comprising: administering a composition comprising polyIC and a cationic polymer; the cationic polymer comprising from about 100 to about 700 amino acid residues; and the mass:mass ratio of the polyIC:cationic polymer is from about 4:1.5 to about 4:3, thereby inducing cytokine production in the cell. The cell may be selected from a peripheral blood mononuclear cell, a dendritic cell or a CD8+ cell. The cytokine may be selected from the group consisting of IL-1α, IL-β, IL-2, 11-4, IL-5, IL-6, IL-7, IL-8, IL-9, IL-10, IL-12 (p70), IL-13, IL-15, IL-17, IL-18, IFNα (alpha), IFNβ (beta), IFNγ (gamma), GM-CSF, TNFα (alpha), G-CSF, MIP-1α (alpha), MIP-113 (beta), MCP-1, EOTAXIN, RANTES, FGF-basic, and VEGF. The polyIC may have an average molecular mass from about 100 to about 5000 kDa. The polycationic polymer may be polyarginine, polylysine, or polyornithine.

In accordance with another aspect of the invention, there is provided a method of inducing an immune response to an immunogen in a subject, comprising: administering a composition comprising an immunogen, polyIC and a cationic polymer; the cationic polymer comprising from about 100 to about 700 amino acid residues; the mass:mass ratio of the polyIC:cationic polymer is from about 4:1.5 to about 4:3; the immune response comprising one or more than one of: a decrease in size of a tumor, a decrease in number of tumors, a decrease insize and number of tumors in the subject; induction of an immune cell, induction of dendritic cell activation, induction of immunogen-specific serum antibody, increase in immunogen-specific serum antibody and induction of cytokine production. The cytokine may be selected from the group consisting of IL-1α, IL-β, IL-2, 11-4, IL-5, IL-6, IL-7, IL-8, IL-9, IL-10, IL-12 (p70), IL-13, IL-15, IL-17, IL-18, IFNα (alpha), IFNβ (beta), IFNγ (gamma), GM-CSF, TNFα (alpha), G-CSF, MIP-1α (alpha), MIP-1β (beta), MCP-1, EOTAXIN, RANTES, FGF-basic, and VEGF. The polyIC may have an average molecular mass from about 100 to about 5000 kDa. The polycationic polymer may be polyarginine, polylysine, or polyornithine. The composition may be administered subcutaneously, intravenously, intraperitoneally, intramuscularly, intranasally, or topically.

The present invention further provides an adjuvant composition comprising polyIC and a cationic polymer; the cationic polymer comprising from about 100 to about 700 amino acid residues, wherein the mass:mass ratio of the polyIC:cationic polymer is from about 4:1.4 to about 4:3. The polyIC may have an average molecular mass from about 100 to about 5000 kDa. The polycationic polymer may be polyarginine, polylysine, or polyornithine. The composition may further comprise a second adjuvant composition. The second adjuvant composition may comprise carboxymethylcellulose, poly-L-lysine, aluminium hydroxide, alum, aluminum trihydrate or other aluminium salts, virosomes, nucleic acids comprising CpG motifs, squalene, oils, saponins, virus-like particles, monophosphoryl-lipidA/trehalose dicorynomycolate, toll-like receptor agonists or copolymers such as polyoxypropylene and polyoxyethylene.

The present invention also provides for a method of stabilizing a polyIC complex comprising mixing polyIC with a cationic polymer comprising from about 100 to about 700 amino acid residues, to obtain a mass:mass ratio of the polyIC:cationic polymer of from about 4:1.5 to about 4:3. The polyIC may have an average molecular mass from about 100 to about 5000 kDa. The polycationic polymer may be polyarginine, polylysine, or polyornithine.

The present invention also provides for a method of making an adjuvant composition comprising combining a solution of polyIC and a solution of a cationic polymer, in a mass:mass ratio of the polyIC:cationic polymer from about 4:1.5 to about 4:3; and wherein the cationic polymer comprising from about 100 to about 700 amino acid residues. The polyIC may have an average molecular mass from about 100 to about 5000 kDa. The polycationic polymer may be polyarginine, polylysine, or polyornithine.

The present invention also provides for an adjuvant composition for topical administration comprising polyIC and a cationic polymer, and a topical base; the cationic polymer comprising from about 100 to about 700 amino acid residues, wherein the mass:mass ratio of the polyIC:cationic polymer is from about 4:1.4 to about 4:3. The polyIC may have an average molecular mass from about 100 to about 5000 kDa. The polycationic polymer may be polyarginine, polylysine, or polyornithine.

The present invention also provides for a method of inducing a localized immune response at or near an epithelial surface comprising applying a composition comprising PolyIC and a cationic polymer, and a topical base to an epithelial surface of a subject, the cationic polymer comprising from about 100 to about 700 amino acid residues, wherein the mass:mass ratio of the polyIC:cationic polymer is from about 4:1.5 to about 4:3

This summary of the invention does not necessarily describe all features of the invention. Other aspects and features of the present invention will become apparent to those of ordinary skill in the art upon review of the following description of specific embodiments of the invention.

### BRIEF DESCRIPTION OF THE DRAWINGS

These and other features of the invention will become more apparent from the following description in which reference is made to the appended drawings wherein:

FIG. 1 shows double-stranded nucleic acid compounds according to Formula Vd and Ve, in accordance with an embodiment of the present invention.

FIG. 2 shows a double-stranded nucleic acid according to Formula VIg, in accordance with an embodiment of the present invention.

FIG. 3 shows a bar graph of IP-10 secretion of HT-29 cells exposed to agonists comprising polyIC at a concentration of 5 ug/ml, according to some embodiments of the invention. The polyIC samples were pretreated by incubation in human serum for 20 hours The Y-axis indicates the IP-10 concentration in pg/ml of the HT-29 supernatant. The X-axis lists the treatments assayed:ICL (4:1.6)—poly IC combined with poly-L-lysine in a 4:1.6 ratio (by mass); ICR (4:1.75)—polyIC combined with polyarginine in a 4:1.75 ratio (by mass); IC—polyIC alone; ICLC-2001—a first lot of poly IC in combination with poly-L-lysine and carboxymethylcelullose; polyL—poly-L-lysine alone; polyR—polyarginine alone; Medium-HS—DMEM with human serum (no agonist).

FIG. 4 shows a bar graph of IP-10 secretion of HT-29 cells exposed to agonists comprising polyIC at a concentration of 1 ug/ml, according to some embodiments of the invention. The polyIC samples were not pretreated with human serum. The Y-axis indicates the IP-10 concentration in pg/ml of the HT-29 supernatant. The X-axis lists the treatments assayed: ICL (4:1.6)—poly IC combined with poly-L-lysine in a 4:1.6 ratio (by mass); ICR (4:1.75)—polyIC combined with polyarginine in a 4:1.75 ratio (by mass); IC—polyIC alone; ICLC-2001—a first lot of poly IC in combination with poly-L-lysine and carboxymethylcelullose; ICLC-2001—a second lot of poly IC in combination with poly-L-lysine and carboxymethylcelullose; Medium-HS—DMEM with human serum (no agonist); Medium-FCS—DMEM with fetal calf serum.

FIG. 5 shows a bar graph of secreted alkaline phosphatase (SEAP) in Human 293 cells stably transfected with human TLR receptors in response to an agonist.

FIG. 6 shows the levels of interferons secreted by donor PBMCs in response to stimulation by polyIC/R or polyICLC. FIG. 6a—interferon alpha secretion; FIG. 6b—interferon beta secretion; FIG. 6c—interferon gamma secretion.

FIG. 7 shows the TLR3 (A) and TLR8 (B) agonist activity of poly IC/R, polyICLC or polyIC, or the individual components polyI, polyC and polyR independently in control HEK293 cells, or HEK293 cells expressing TLR3 or TLR8 receptors.

FIG. 8 shows a graph of the rate of RNAse A hydrolysis of polyIC (solid diamond); polyIC:lysine (4:1.6 mg/ml—solid square); polyIC:lysine (4:2 mg/ml—solid triangle); polyIC:Arginine (4:1.5 mg/ml—cross); poly IC:arginine (4:2 mg/ml—cross with diamond); polyICLC (solid circle). A₂₆₀ is along the Y-axis, time along the X-axis.

FIG. 9 shows a bar graph of IP-10 secretion of HT-29 cells exposed to agonist mixtures comprising poly IC in combination with polyarginine (polyR) and/or an immunogen (OVA); the polyIC, polyR and/or OVA being combined in varying orders, according to some embodiments of the invention. The agonist mixtures were pretreated with 20% human serum for 20 hours before adding to the cells; HT-29 cells were exposed to the agonist mixtures for 24 (small hatch-marks) or 48 (large hatch-marks) hours.

FIG. 10 shows a bar graph of IP-10 secretion of HT-29 cells exposed to agonist mixtures comprising poly IC in combination with polyarginine (polyR) and/or an immunogen (OVA); the polyIC, polyR and/or OVA being combined in varying orders, according to some embodiments of the invention. The agonist mixtures were pretreated with 20% human serum for 20 hours before adding to the cells; HT-29 cells were exposed to the agonist mixtures for 24 (small hatch-marks) or 48 (large hatch-marks) hours.

FIG. 11 shows murine interferon-gamma ELISpot data from restimulated splenocytes from naïve mice, or mice immunized with HspE7 (500 ug) alone or in combination with and polyIC/R (100 ug). Restimulation of splenocytes was with media, an unrelated antigen (influenza NP) or the E7₄₉₋₅₇ peptide fragment, followed by restimulation of splenocytes with. Experimental treatments are indicated along the X-axis; the Y-axis shows spot-counts (stained cells) per 2×10⁵ splenocytes.

FIG. 12 shows a graph and table of percentages of tumor incidence in mice implanted with TC-1K cells. Naïve, HspE7 and ICR (100 ug) treated mice demonstrate 100% tumor incidence from day 0 to day 17; HspE7+ICR (100 ug), HspE7+ICR (50 ug) and HspE7+ICR (25 ug) demonstrate 100% tumor incidence from day 0 to day 9, decreasing to 0% from day 11 onward.

FIG. 13A-F shows graphs of selected cytokine levels in culture medium of human PBMCs treated ex-vivo with TLR-3 agonist compositions according to some embodiments of the invention. X-axis—TLR-3 agonist compositions controls at concentrations ranging from 1.6 to 50 ug/ml; Y-axis—cytokine concentration (pg/ml): A—IL-6; B—IL-8; C—IL-10; D—IL-12 p70; E—TNF alpha; F—IFN gamma.

FIG. 14 shows a graph of tumor prevention (prophylactic treatment) in a murine TC-1K tumor model. X axis is days post tumor implantation; Y axis is percent tumor free. A indicates start point of treatment; B indicates start point of tumor cell implantation. Solid diamond, naïve mice; solid square, HspE7 treated mice; solid triangle, IC/R treated mice; HspE7+polyIC/R treated mice −25 ug poly IC/R (solid circle), 50 ug poly IC/R (*), 100 ug poly IC/R (X). Prophylactic treatment was stated on day 0, and tumor cells implanted on day 7.

FIG. 15 shows murine IgG2c antibody response against HspE7 using poly IC/R in the presence or absence of alum. X axis—specific treatments; Y axis—absorbance.

FIG. 16 shows a graph of tumor regression in a murine TC-1K tumor model. X axis is days post tumor implantation; Y axis is percent tumor positive. A indicates start point of treatment; B indicates start point of tumor cell implantation. Solid diamond, naïve mice; solid square, HspE7 treated mice; solid triangle, IC/R treated mice; solid circles HspE7+polyIC/R treated mice (25, 500 or 100 ug polyIC/R). Tumor cells were implanted at day 1, and treatment was started at day 7.

FIG. 17 shows the murine IgG2c antibody response against HspE7 using poly IC/R as per FIG. 15, but with the HspE7+PolyIC/R+Alum data omitted to illustrate the difference in the other treatments. X axis—specific treatments; Y axis—absorbance.

### DETAILED DESCRIPTION

The present invention relates to the field of immunology, and immunostimulatory agents. More specifically, the present invention provides compositions comprising polycationic polymers and immunostimulatory nucleic acids.

In the description that follows, a number of terms are used extensively, the following definitions are provided to facilitate understanding of various aspects of the invention. Use of examples in the specification, including examples of terms, is for illustrative purposes only and is not intended to limit the scope and meaning of the embodiments of the invention herein.

The present invention provides a composition comprising polyI and polyC, or polyA and polyU oligonucleotide polymers, wherein each of the oligonucleotide polymer comprises at least one locked nucleic acid (LNA) residue. The dsRNA may be comprised of equimolar quantities of polyI and polyC oligonucleotide polymers (polyI:C), or equimolar quantities of polyA and polyU oligonucleotide polymers (polyA:U).

The dsRNA of the present invention, that comprise one or more than one LNA, may be used for a variety of purposes, for example, but not limited to their use as adjuvants, or as therapeutic agents. For example the dsRNA that comprise one or more than one LNA may be a polyI:C compound comprising one or more than one LNA.

The present invention further provides a composition comprising oligonucleotide polymers comprising at least one CpG motif and at least one LNA residue, and a combination of I and C residues, or combination A and U residues. The oligonucleotide polymers may hybridize and form double-stranded molecules, for example double-stranded RNA (dsRNA). For example the dsRNA that comprise a CpG motif and having one or more than one LNA may be a polyI:C compound comprising one or more than one LNA. The dsRNA may be comprised of equimolar quantities of polyI and polyC oligonucleotide polymers (polyI:C), or equimolar quantities of polyA and polyU oligonucleotide polymers (polyA:U). In another example, the oligonucleotide polymers may comprise a CpG motif having one, or more than one LNA, and a mixture of I and C nucleosides, or a mixture of A and U nucleosides, wherein the CpG motif and the I and C nucleosides of each oligonucleotide in the pair are arranged so as to hybridize to form a double-stranded molecule

The dsRNA of the present invention, that comprise at least one CpG motif and one or more than one LNA, may be used for a variety of purposes, for example, but not limited to their use as adjuvants, or as therapeutic agents. For example the dsRNA that comprise at least one CpG motif and one or more than one LNA may be a polyI:C compound comprising one or more than one LNA.

The present invention also provides a composition comprising mismatched dsRNA, wherein each of the oligonucleotide polymer comprises at least one LNA residue. The dsRNA may be, for example a modification of polyI:poly(C₁₂U) (AMPLIGEN™), wherein at least one, or more than one, of the nucleic acids is an LNA. The mismatched dsRNA may be combined with other dsRNAs, including those comprising a CpG motif.

Immunostimulatory agents are compounds or compositions that initiate an immune response, or provide a catalytic effect in initiating an immune response. The immune response may be solely an innate (or non-adaptive) immune response, such as inducing the production and secretion of cytokines (for example interferons, interleukins, colony stimulating factors and the like) which in turn incite phagocytic cells to migrate and ingest foreign immunogens nonspecifically and present the immunogens for recognition by the adaptive immune system. Alternatively, the immune response may be an adaptive immune response, in response to the presence of particular immunogens (such as those presented by an phagocytic cell, also referred to as an antigen-presenting cell).

Use of the term ‘a’ or ‘an’ includes both singular and plural references.

An adjuvant is an immunostimulatory agent that has no antigen- or immunogen-specific effect by itself, but stimulates the immune system to increase the response to a specific immunogen, or group of immunogens. An adjuvant may alternately be referred to as an “immune response modifier” (IRM). The ability of an immunogen to induce a response of the innate or adaptive immune system is referred to as the “biological activity” of the immunogen. An adjuvant may mediate, augment or stimulate the biological activity of an immunogen. In some examples, the immunogen may have very little or negligible biological activity in the absence of an adjuvant or adjuvant composition. An adjuvant composition may comprise one, or more than one immunostimulatory agents.

In other examples, an adjuvant or adjuvant composition may have an immunogenic effect that is independent of a specific antigen. For example, adjuvant compositions may induce maturation of some immune cells, or may induce clonal expansion of some immune cells, or may induce cytokine production in some immune cells. Examples of immune cells include peripheral blood mononuclear cells (PBMC), granulocytes (CD15+), monocytes, (CD14+), T-lymphocytes (CD3+), T helper cells (CD4+), cytotoxic T cells (CD8+), B lymphocytes (CD 19+, CD20+), dendritic cells and natural killer cells (CD16+, CD56+).

The biological activity of an adjuvant composition, an immunogen, or an adjuvant composition and an immunogen in combination may be measured by any of several assays known in the art. Alternately, the immunostimulatory effect of an adjuvant in combination with an immunogen may also be assessed in a similar assay. For example, induction of antigen-specific CD8-positive T lymphocytes may be quantified through use of an ELISPOT assay (Asai et al 2000 Clin. Diag. Lab Immunol 7:145-154). Other versions of an ELISPOT assay may be used for other cytokines, see, for example, Kalyzhny et al 2005. Methods Mol Biol 302:15-31; Ott, et al. J. Immunol. Methods. 2004 Feb. 15; 285(2):223-35; Forsthuber, et al. Science, 271: 1728-1730. Other T-cell assays that may be useful for monitoring a response to an immunogen include intracellular cytokine flow cytometry, proliferation assays, antibody microarrays, and the like. See, for example Nagorsen et al 2004. Expert Opin Biol Ther 4:1677-84, or Handbook of Experimental Immunology, Vols. 1-IV, D. M. Weir and C. C. Blackwell, eds., 1986, Blackwell Scientific Publications. Interferon-α (alpha), β (beta) and γ (gamma) may be quantified, for example, with an Interferon ELISA kit (Kim et al 2004. Nature Biotechnology 22:321-325), or an ELISpot kit (R&D Systems Catalogue # EL485) Multiplexed assays, for example, bead-based systems (Luminex, Panomics and the like) allow for simultaneous quantification of a plurality of cytokines, and are available from various suppliers (e.g. R&D Systems, Millipore and the like). Examples of cytokines include IL-1α, IL-1β, IL-2, 11-4, IL-5, IL-6, IL-7, IL-8, IL-9, IL-10, IL-12 (p70), IL-13, IL-15, IL-17, IL-18, IFNα (alpha), IFNβ (beta), IFNγ (gamma), GM-CSF, TNFα (alpha), G-CSF, MIP-1α (alpha), MIP-1β (beta), MCP-1, EOTAXIN, RANTES, FGF-basic, VEGF and the like. For clarity, the term ‘cytokine’ includes alternative nomenclatures such as lymphokines, interleukins, or chemokines. Antigen-specific antibodies may be detected and/or quantified using any of several assays known in the art. Examples include ELISA, western blot, flow-cytometry or bead-based methods such as RapidQuant™ (Guava Technologies) or the like. Antibodies may be of several isotypes or subtypes, such as IgA, IgM, IgG, IgD and IgE, with particular isotypes or subtypes being predominant in certain tissues, in response to type of pathogens (bacterial, viral, parasite or protozoan) and/or at certain stages in the immune response.

Alternately, an adjuvant composition may have biological activity independent of an immunogen. For example, cytokine production may be induced by administering an adjuvant composition according to some embodiments of the invention to a subject, to provide a therapeutic effect in the subject to whom it is administered.

Biological activity, including cytokine production, may be assessed with regards to the subject as a whole (e.g. via a serum, blood or other fluid or tissue sample), or with regards to cells, or a particular cell type. The cells may be, for example, peripheral blood mononuclear cells (PBMCs) or particular immune cells, such as CD8+ cells.

The invention, therefore, provides for methods of inducing cytokine production in a cell in the absence of an immunogen. The invention further provides for methods of inducing immune cells, including dendritic and CD8+ cells. The invention further provides for methods of inducing antibody production—without wishing to be bound by theory, such inducing of antibody production may be a result of inducing B lymphocytes or other immune cells that stimulate B lymphocytes.

In other examples, an IP-10 assay may be used to assess the ability of an adjuvant composition to provide TLR-3 agonist activity. Human HT29 cells secrete IP-10 into the culture supernatant as a result of stimulation with a TLR-3 agonist. IP-10 in the culture supernatant may be quantified, by, for example, ELISA. As another example, peripheral blood mononuclear cells (PBMCs) secrete cytokines into the supernatant as a result of stimulation with a TLR-3 agonist. The secreted cytokines, for example interferon-alpha,-beta and/or -gamma may be quantified by, for example ELISA, ELISpot, or the like. As another example, the maturation of immune effector cells, such as dendritic cells, may be assessed.

The terms “subject” and “patient” may be used interchangeably. A “subject” refers to an animal, or a mammal, including, but not limited to, a mouse, rat, dog, cat, pig, or primate, including but not limited to a monkey, chimpanzee or human. The subject may be immunologically naïve with respect to a particular immunogen or group of immunogens, or the subject may have been previously exposed to a particular immunogen or group of immunogens. Previous exposure may have resulted from, for example, deliberate immunization with a particular immunogen or group of immunogens, exposure to an infectious agent comprising a particular immunogen or group of immunogens, or cross-reactive exposure to a first immunogen or group of immunogens, that allows an immune response to a second immunogen or group of immunogens. The second immunogen or group of immunogens may be similar to, the same as, or different from the first immunogen or group of immunogens.

As used herein, the term “LNA-modified oligonucleotide” includes to any oligonucleotide either fully or partially modified with one or more LNA monomer. Thus, an LNA-modified oligonucleotide may be composed entirely by LNA monomers, or a LNA-modified oligonucleotide may comprise one LNA monomer.

The term “DNA monomer” refers to a deoxyribose sugar bonded to a nitrogenous base, while the term “RNA monomer” refers to a ribose sugar bonded to a nitrogenous base. Examples of DNA monomers that may comprise compositions according to various embodiments of the present invention include, but are not limited to, deoxyadenosine, deoxyguanosine, deoxythymidine, deoxyuridine, deoxycytidine, deoxyinosine and the like. Examples of RNA monomers that may comprise compositions according to various embodiments of the present invention include, but are not limited to, adenosine,guanosine, 5-methyluridine, uridine, cytidine, inosine, and the like. Other DNA or RNA monomers according to various embodiments of the present invention may comprise other nitrogenous bases, as are known in the art.

As used herein, the term “LNA monomer” typically refers to a nucleoside having a 2′-4′ cyclic linkage as described in U.S. Pat. No. 6,268,490, U.S. Pat. No. 679,449, U.S. Pat. No. 7,034,133 (each of which are incorporated herein by reference). Bicyclic nucleosides (see below) may provide conformational restriction to the oligonucleotide, and may provide varying hybridization or stability profiles compared to unmodified oligonucleotides.

The term ‘nucleoside’ refers to a molecule of ribose or deoxyribose sugar bonded through carbon-1 of the sugar ring to a nitrogenous base. Examples of nitrogenous bases include purines such as adenine, guanine, 6-thioguanine, hypoxanthine, xanthine, and pyrimidines such as cytosine, thymine and uracil. Examples of purine nucleosides include adenosine (A), guanosine (G), inosine (I), 2′-methyl-inosine, 2′-methyl-adenosine, 2′-methyl-guanine, 2-chlorodeoxyadenosine, 7-halo-7-deaza-adenosine, 7-halo-7-deaza-guanine, 7-propyne-7-deaza adenosine, 7-propyne-7-deaza-guanine, 2-amino-adenosine, 7-deazainosine, 7-thia-7,9-dideazainosine, formycin B, 8-Azainosine, 9-deazainosine, allopurinol riboside, 8-bromo-inosine, 8-chloroinosine, 7-deaza-2-deoxy-xanthosine, 7-Deaza-8-aza-adenosine, 7-deaza-8-aza-guanosine, 7-deaza-8-aza-deoxyadenosine, 7-deaza-8-aza-deoxyguanosine, 7-deaza-adenosine, 7-deaza-guanosine, 7-deaza-deoxyadenosine, 7-deaza-deoxyguanosine, 8-amino-adenosine, 8-amino-deoxyadenosine, 8-amino-guanosine, 8-amino-deoxyguanosine,3-deaza-deoxyadenosine, 3-deaza-adenosine, 6-thio-deoxyguanosine, and the like, and other purine nucleosides as described in Freier et al 1997 (Nucleic Acids Res. 25:4429-4443), incorporated herein by reference.

Examples of pyrimidine nucleosides include deoxyuridine (dU), uridine (U), cytidine (C), deoxycytidine (dC), thymidine (T), deoxythymidine (dT), 5-fluoro-uracil, 5-bromouracil, 2′-O-methyl-uridine, 2′-O-methyl cytidine, 5-iodouracil, 5-methoxy-ethoxy-methyl-uracil, 5-propynyl deoxyuridine, pseudoisocytidine, 5-azacytidine, 5-(1-propynyl)cytidine, 2′-deoxypseudouridine, 4-thio-deoxythymidine, 4-thio-deoxyuridine, and the like, and other substituted pyrimidines as disclosed in Freier, et al, 1997 (Nucleic Acids Res. 25:4429-4443).

Purine or pyrimidine nucleosides also include phosphoramidite derivatives used in oligonucleotide synthesis using standard methods.

The term nucleoside further includes bicyclic nucleoside analogues according to Formula (I), as described in, for example, U.S. Pat. No. 6,268,490 (which is incorporated by reference):

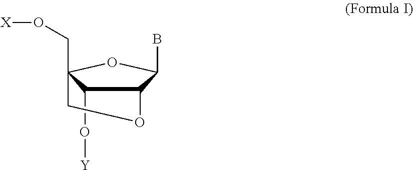

B may be any nitrogenous base, for example a pyrimidine or purine nucleic acid base, or an analogue thereof.
X and Y may be identical or different, and may be any internucleoside linkage group.

Such bicyclic nucleoside analogues may alternately be referred to as “locked nucleic acid monomer’ or “locked nucleoside monomer” or “LNA monomer” or “LNA residue”. Methods of synthesis and polymerization of nucleic acid polymers comprising LNA monomers are described in, for example, WO 99/14226, WO 00/56746, WO 00/56748, WO 01/25248, WO 0148190, WO 02/28875, WO 03/006475, WO 03/09547, WO 2004/083430, U.S. Pat. No. 6,268,490, U.S. Pat. No. 679,449, U.S. Pat. No. 7,034,133 (each of which are herein incorporated by reference).

Other examples of nucleoside analogues, as disclosed in WO 01/048190 (which is incorporated herein by reference) include non-LNA bicyclic nucleosides, for example, but not limited to:
bicyclo[3.3.0] nucleosides with an additional C-3′,C-5′-ethanobridge;
bicarbocyclo[3.1.0] nucleosides with an additional C-1′,C-6′- or C-6′,C-4′ methano bridge
bicyclo[3.3.0]- and [4.3.0] nucleosides containing an additional C-2′,C-3′dioxalane ring synthesised as a dimer with an unmodified nucleoside where the additional ring is part of the internucleoside linkage replacing a natural phosphordiester linkage; dimers containing a bicyclo[3.1.0] nucleoside with a C-2′,C-3′-methano bridge as part of amide- and sulfonamide-type internucleoside linkages;
bicyclo[3.3.0] glucosederived nucleoside analogue incorporated in the middle of a trimer through formacetal internucleoside linkages;
tricyclo-DNA in which two five membered rings and one three membered ring constitute the backbone;
1,5-Anhydrohexitol nucleic acids; and
bicyclic[4.3.0]- and [3.3.0] nucleosides with additional C-2′,C-3′-connected six and five-membered ring.

“Nucleoside” also includes nucleosides having substituted ribose sugars (bicyclic or otherwise). Examples of substituted ribose sugars are described in, for example, Freier, 1997 (Nucleic Acids Res. 25:4429-4443), which is incorporated by reference).

A ‘nucleotide’ refers to a nucleoside having an internucleoside linkage group bonded through the carbon-5 of the sugar ring, usually a mono-, di- or tri-phosphate. An oligonucleotide ‘backbone’ refers to, for example, in a naturally occurring nucleic acid, the alternating ribose/phosphate chain covalently bonded through the carbon-5 and carbon-3 of the sugar, formed by polymerization of a population of nucleotides. This polymerization may be enzymatic, or may involve synthetic chemical methods, as are known in the art. See, for example, Gait, pp. 1-22; Atkinson et al., pp. 35-81; Sproat et al., pp. 83-115; and Wu et al., pp. 135-151, in Oligonucleotide Synthesis: A Practical Approach, M. J. Gait, ed., 1984, IRL Press, Oxford; or Molecular Cloning: a Laboratory Manual 3^{rd} edition. Sambrook and Russell. CSHL Press, Cold Spring Harbour, N.Y. (both of which are herein incorporated by reference).

The DNA or RNA, or LNA monomers may be, for example, mono-, di- or tri-phosphate nucleotides suitable for enzymatic polymerization. In other examples, the DNA or RNA, or LNA monomers may be phosphoramidites, suitable for non-enzymatic polymerization or synthesis of nucleic acid polymers.

An internucleoside linkage group refers to a group capable of coupling two nucleosides, as part of an oligonucleotide backbone. Examples of internucleoside linkage groups are described by Praseuth et al (Biochimica et Biophysica Acta 1489:181-206, incorporated herein by reference), including phosphodiester (PO₄—), phosphorothioate (PO3_{S}-), phosphoramidate (N3′-P5′) (PO₃NH) and methylphosphonate (PO₃CH₃), peptidic linkages (“PNA”), and the like.

The terms “nucleotide polymer”, “oligonucleotide”, “nucleic acid”, or “nucleic acid polymer” are used interchangeably, and refer to polymers comprising at least two nucleotides. The nucleotide polymer may comprise a single species of DNA monomer, RNA monomer, or may comprise two or more species of DNA monomer, RNA monomers in any combination. Nucleic acid may be single or double-stranded, for example, a double-stranded nucleic acid molecule may comprise two single-stranded nucleic acids that hybridize through base pairing of complementary bases.

A “polyI” oligonucleotide includes a majority of inosine, inosine-analogue nucleosides, or a combination thereof. Inosine-analogue nucleosides include, for example, 7-Deazainosine, 2′-O-methyl-inosine, 7-thia-7,9-dideazainosine, formycin B, 8-Azainosine, 9-deazainosine, allopurinol riboside, 8-bromo-inosine, 8-chloroinosine and the like.

A “polyC” oligonucleotide includes a majority of cytidine, cytidine-analogue nucleosides, or a combination thereof. Cytidine-analogue nucleosides include, for example, 5-methylcytidine, 2′-O-methyl-cytidine, 5-(1-propynyl)cytidine, and the like.

A “polyA” oligonucleotide includes a majority of adenosine, adenosine-analogue nucleosides, or a combination thereof. Adenosine-analogue nucleosides include, for example, 2-amino-ademosine, 2′-O-methyl-adenosine, 2-amino-deoxyademosine, 7-deaza-2′-adenosine, 7-deaza-2′-deoxyadenosine, and the like.

A “polyU” oligonucleotide includes a majority of uridine, uridine-analogue nucleosides, or a combination thereof. Uridine-analogue nucleosides include, for example deoxyuridine (dU), cytidine (C), deoxycytidine (dC), thymidine (T), deoxythymidine (dT), 5-fluoro-uracil, 5-bromouracil, 2′-O-methyl-uridine, 5-iodouracil, 5-methoxy-ethoxy-methyl-uracil, 5-propynyl deoxyuridine, and the like.

A “CpG motif” or a “CpG element” or a “CpG site” refers to a nucleotide motif comprising a cytosine nucleoside occurring adjacent to a guanine nucleoside in a nucleic acid. The nucleosides C and G are separated by a phosphate which links the two together in a conventional 5′-3′ nucleosidic linkage. A CpG motif may be described generally as XnCpGXn, where X is any nucleoside and n is any number from 1 to about 500 or any amount therebetween, for example from about 1 to about 300 or any amount therebetween, from about 1 to about 250 or any amount therebetween, from about 1 to about 200 or any amount therebetween, from about 1 to about 150 or any amount therebetween, or from 1, 5, 10, 20, 30, 40, 50, 60, 70, 80, 90, 100, 125, 150, 175, 200, 225, 250, 275, 300, 325, 250, 275, 400, 425, 250, 475, 500 or any amount therebetween. As described herein, it is preferred that one or more than one of the nucleosides, C, G, within the CpG motif is an LNA

The strands of double-stranded nucleic acid molecules, including dsRNA, interact in an ordered manner through hydrogen bonding—also referred to as ‘Watson-Crick’ base pairing. Variant base-pairing may also occur through non-canonical hydrogen bonding includes Hoogsteen base pairing. Under some thermodynamic, ionic or pH conditions, triple helices may occur, particularly with ribonucleic acids. These and other variant hydrogen bonding or base-pairing are known in the art, and may be found in, for example, Lehninger—Principles of Biochemistry, 3^{rd} edition (Nelson and Cox, eds. Worth Publishers, New York.), herein incorporated by reference.

PolyI and polyC, or polyA and polyU oligonucleotides according to various embodiments of the invention and under suitable temperature, ionic and pH conditions may form double-stranded complexes through Watson-Crick hydrogen bonding. The particular temperature, ionic and pH conditions suitable for such complex formation are discernable by one of skill in the art—examples of methods, calculations, techniques and the like for discerning such conditions may be found in, for example, Freier, (1997, Nucleic Acids Res. 25:4429-4443; which is incorporated herein by reference). The formation of such double-stranded complexes may alternately be referred to as ‘hybridization’.

Double stranded RNA (dsRNA) molecules according to various embodiments of the invention that contain at least one LNA, are generally described by Formula II:

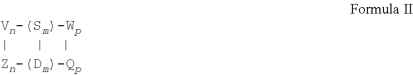

Formula II represents a double-stranded RNA molecule having a first strand Vₙ—(Sₘ)—Wₚ and a second strand Zₙ—(Dₘ)—Qₚ, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

where:
n is any integer from 1 to 5, or any amount therebetween;
p is any integer from 1 to 5, or any amount therebetween;
V, W, Z and Q is any nucleoside, ribonucleoside, deoxyribonucleoside, nucleoside analogue, ribonucleoside analogue or deoxyribonucleoside analogue;
m is any integer from 1 to 500, or any amount therebetween;
S is inosine, an inosine-analogue nucleoside, adenine or an adenine-analogue nucleoside;
D is cytosine, a cytosine-analogue nucleoside, uracil, or a uracil-analogue nucleoside;
wherein one or more than one of V, S, W, Z, D, and Q, comprises one or more than one locked nucleic acid (LNA) monomer.

The present invention also provides a dsRNA compound of Formula II where S and D are I and C as defined below (Formula IIa):

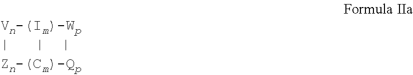

Formula IIa represents a double-stranded RNA molecule having a first strand Vₙ—(Iₘ)—Wₚ and a second strand Zₙ—(Cₘ)-Qₚ, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).
n and p may independently be any integer from 1 to 5 or any amount therebetween;
V, W, Z and Q may independently be any nucleoside connected by an internucleoside linkage group, where V and Z are capable of bonding, and W and Q are capable of bonding;
m may be any integer from 1 to 500, or 10-50, or any integer therebetween, including 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100;
I is inosine, or any inosine-analogue nucleoside connected to V, W and to geminal inosine or inosine-analogues nucleoside by an internucleoside linkage group;
C is cytosine, or any cytosine-analogue nucleoside connected to V, W and to geminal cytosine, or any cytosine-analogues nucleoside by an internucleoside linkage group; and
wherein one or more than one of V, I, W, Z, C, and Q, comprises one or more than one LNA monomer.

Alternate dsRNA molecules of the present invention, include a compound of Formula II, where S and D are I and C, and further comprising R, as defined below (Formula IIb):

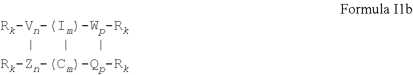

Formula IIb represents a double-stranded RNA molecule having a 5′, a 3′, or both a 5′ and 3′ overhanging base, and having a first strand Rₖ—Vₙ—(Iₘ)—Wₚ—Rₖ and a second strand Rₖ—Zₙ—(Cₘ)-Qₚ-Rₖ, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right), where;
V, W, Z, Q, I, C, n, p and m are as described for Formula IIa;
k may be any integer from 0 to 10 inclusive, or any integer therebetween.
R may independently be any ribonucleoside connected by an internucleoside linkage group to the geminal nucleoside, or R may be absent. In some embodiments, for example, a 5′ R ribonucleoside of the first strand is capable of bonding with a 3′ R ribonucleoside of the second strand; and
wherein one or more than one of R, V, I, W, Z, C, and Q, comprises one or more than one LNA monomer.

Double stranded RNA (dsRNA) molecules that contain at least one LNA, include a compound of Formula II, where S and D are A and U, as defined below (Formula IIc) are generally are also described by Formula IIc:

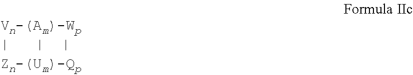

Formula IIc represents a double-stranded RNA molecule having a first strand Vₙ-(Aₘ)-Wₚ and a second strand Zₙ—(Uₘ)-Qₚ, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right), where;
V, W, Z, Q, n, p and m are as described for Formula IIa;
A may be adenosine, or any adenosine-analogue nucleoside connected to V, W and to geminal adenosine or adenosine-analogues by an internucleoside linkage group;
U may be uridine, or any uridine-analogue nucleoside connected to V, W and to geminal uridine, or any uridine-analogues by an internucleoside linkage group; and
wherein one or more than one of R, V, A, W, Z, U, and Q, comprises one or more than one LNA monomer.

Alternate dsRNA molecules of the present invention, include a compound of Formula II, where S and D are A and U, and further comprising R, as defined below (Formula IId): where at least one nucleoside for the dsRNA is an LNA

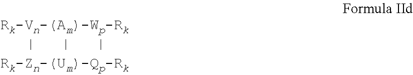

Formula IId represents a double-stranded RNA molecule having a 5′, a 3′, or both a 5′ and 3′ overhanging base, and having a first strand Rₖ—Vₙ—(Iₘ)—Wₚ—Rₖ and a second strand Rₖ—Zₙ—(Cₘ)-Qₚ-Rₖ, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right), where;

V, W, Z, Q, I, C, R, k, n, p and m are as described for Formula IIa;

A and U are as described for Formula IIc; and

wherein one or more than one of R, V, A, W, Z, U, and Q, comprises one or more than one LNA monomer.

Compounds according to Formula II, IIa, IIb, IIc, IId may comprise one or more than one LNA molecule at one or more than one of the R, V, W, Z, Q. For example one or more than one LNA molecule may be positioned at the 5′ end of Formula II, IIa, IIb, IIc, or IId, within V, Q, or both V and Q, one or more than one LNA molecule may be positioned at the 3′ end of Formula II, IIa, IIb, IIc or IId, within Z, W, or both Z and W, or one or more than one LNA molecule may be positioned at the 5′ and the 3′ ends of Formula II, IIa, IIb, IIc or IId, within V, W, Z, Q or a combination thereof.

The present invention also provides a compound according to Formula II, IIa, IIb, IIc, or IId, where V and W are LNA nucleosides (V_{LNA}, W_{LNA}, respectively), Z and Q are LNA nucleosides (Z_{LNA}, Q_{LNA}, respectively), I is inosine, C is cytidine, n and p is 2, m is as defined above, and may be from about 1 to about 500 or any amount therebetween, for example m is from about 10 to about 50 or any amount therebetween, for example m is about 1, 2, 5, 7, 10, 12, 14, 16, 17, 18, 19, 20, 21, 22, 23, 24, 25, 26, 27, 28, 29, 30, 32, 35, 40, 45, 50, 60, 70, 80 90, 100 or any amount therebetween, for example m may be 18, 19, 20, 21, 22, 23, 24, 25, and the internucleoside linkage groups therebetween are phosphodiester. A non-limiting example of this compound is shown in Formula III:

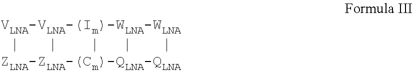

A non-limiting example of a dsRNA of the present invention may be as shown in any one of Formula IIIa, IIIb, IIIc, or IIId, where G is a guanosine nucleoside, C is a cytidine nucleoside and m is 22:

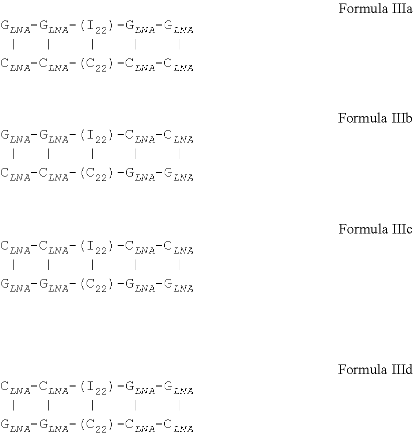

Single stranded nucleic acid molecules, or single-stranded RNA (ssRNA) molecules according to various embodiments of the invention that comprise at least one LNA, are generally described by Formula IVa:

| | |
|---|---|
| Vₙ-(Sₘ)-Wₚ | Formula IVa |

Formula IVa represents a single-stranded nucleic acid molecule having a configuration Vₙ—(Sₘ)—Wₚ, represented in a 5′ to 3′ direction (left to right), where

V, W, n, p and m are as in Formula IIa;

S is inosine, an inosine-analogue nucleoside, adenine or an adenine-analogue nucleoside, and;

wherein one or more than one of V, S, and W comprises one or more than one locked nucleic acid (LNA) monomer.

Single stranded nucleic acid molecules, or single-stranded RNA (ssRNA) molecules according to various embodiments of the invention that comprise at least one LNA, are generally described by Formula IVb:

| | |
|---|---|
| Qₚ-(Dₘ)-Zₙ | Formula IVb |

Formula IVb represents a single-stranded RNA molecule having a first strand Qₚ-(Dₘ)-Zₙ, represented in a 5′ to 3′ direction (left to right) where:
Z, Q, n, p and m are as described for Formula IIa;

D is cytosine, a cytosine-analogue nucleoside, uracil, or a uracil-analogue nucleoside; and

wherein one or more than one of Z, D, and Q, comprises one or more than one locked nucleic acid (LNA) monomer.

In some embodiments of the invention, compositions may comprise single-stranded RNA molecules according to Formula IVa or Formula IVb, or both Formula IVa and Formula IVb in various molar ratios. For example, in some embodiments, single stranded RNA molecules according to Formula IVa and Formula IVb may be combined in about equimolar ratios. Some, none or all single-stranded RNA molecules according to Formula IVa and Formula IVb may hybridize with another complementary single-stranded RNA molecule to form double-stranded RNA molecules.

In other embodiments, single stranded RNA molecules according to Formula IVa may be combined in a composition with single stranded RNA molecules according to Formula IVb in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVa or Formula IVb may hybridize with another complementary single-stranded RNA molecule to form double-stranded RNA molecules.

In other embodiments, single stranded RNA molecules according to Formula IVb may be combined in a composition with single stranded RNA molecules according to Formula IVa in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVa or Formula IVb may hybridize with another complementary single-stranded RNA molecule to form double-stranded RNA molecules.

Single stranded nucleic acid molecules, or single-stranded RNA (ssRNA) molecules according to various embodiments of the invention that comprise at least one LNA, are generally described by Formula IVc:

| | |
|---|---|
| Rₖ-Vₙ-(Sₘ)-Wₚ-Rₖ | Formula IVc |

Formula IVc represents a single-stranded nucleic acid molecule having a configuration Rₖ—Vₙ—(Sₘ)—Wₚ—Rₖ, represented in a 5′-3′ direction (left to right) where:

V, W, n, p and m are as described for Formula IIa

S is inosine, an inosine-analogue nucleoside, adenine or an adenine-analogue nucleoside;

—R and k are as described for Formula IIb; and

wherein one or more than one of V, S, R and W comprises one or more than one locked nucleic acid (LNA) monomer.

Single stranded nucleic acid molecules, or single-stranded RNA (ssRNA) molecules according to various embodiments of the invention that comprise at least one LNA, are generally described by Formula IVd:

| | |
|---|---|
| Rₖ-Qₚ-(Dₘ)-Zₙ-Rₖ | Formula IVd |

Formula IVd represents a single-stranded nucleic acid molecule having a configuration Rₖ-Qₚ-(Dₘ)-Zₙ-Rₖ represented in a 5′-3′ direction (left to right) where:
Z, Q, n, p and m are as described for Formula IIa
R and k are as described for Formula IIb; and
D is cytosine, a cytosine-analogue nucleoside, uracil, or a uracil-analogue nucleoside; and
wherein one or more than one of R, Z, D, and Q, comprises one or more than one locked nucleic acid (LNA) monomer.

In some embodiments of the invention, compositions may comprise single-stranded RNA molecules according to Formula IVc or Formula IVd, or both Formula IVc and Formula IVd in various molar ratios. For example, in some embodiments, single stranded RNA molecules according to Formula IVc and Formula IVd may be combined in about equimolar ratios. Some, none or all single-stranded RNA molecules according to Formula IVc and Formula IVd may hybridize with another complementary single-stranded RNA molecule to form double-stranded RNA molecules.

In other embodiments, single stranded RNA molecules according to Formula IVc may be combined in a composition with single stranded RNA molecules according to Formula IVd in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVc or Formula IVd may hybridize with another complementary single-stranded RNA molecule to form double-stranded RNA molecules.

In other embodiments, single stranded RNA molecules according to Formula IVd may be combined in a composition with single stranded RNA molecules according to Formula IVc in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVc or Formula IVd may hybridize with another complementary single-stranded RNA molecule to form double-stranded RNA molecules.

Non-limiting examples of single-stranded nucleic acids of the present invention may be as shown in any one of Formula IVe, IVf, IVg, IVh, IVi, or IVj, (shown in a 5′-3′ orientation, left to right), where I is a 2′-O-methyl-inosine nucleoside, C is a 2′-O-methyl-cytosine nucleoside, G is a 2′-O-methyl-guanosine nucleoside, T is a 2′-O′ methyl-thymidine nucleoside, A is a 2′-O-methyl-adenosine nucleoside, u IS A 2′-O-methyl-uridine nucleoside, T_{LNA} is an thymidine nucleoside with an LNA ribose, G_{LNA} is a guanosine nucleoside with an LNA ribose, C_{LNA} is a cytosine nucleoside with an LNA ribose, A_{LNA} is an adenosine nucleoside with an LNA ribose, and m is 15:

| | |
|---|---|
| (I₁₅)-G-T_{LNA}-G_{LNA}-A-T_{LNA}-A-T_{LNA}-G_{LNA} | Formula IVe |
| (C₁₅)-C_{LNA}-A-T_{LNA}-A-T_{LNA}-C-A_{LNA}-C_{LNA} | Formula IVf |
| G_{LNA}-(I₁₅)-G-T_{LNA}-G_{LNA}-A-T_{LNA}-A-T_{LNA} | Formula IVg |
| C_{LNA}-(C₁₅)-C_{LNA}-A-T_{LNA}-A-U-C_{LNA}-A_{LNA} | Formula IVh |
| T_{LNA}-G_{LNA}-(I₁₅)-T_{LNA}-T_{LNA}-A-T_{LNA}-A_{LNA} | Formula IVi |
| A_{LNA}-C_{LNA}-(C₁₅)-C_{LNA}-A-T_{LNA}-A-T_{LNA}-C_{LNA} | Formula IVj |

In some embodiments of the invention, compositions may comprise single-stranded RNA molecules according to Formula IVe or Formula IVf, or both Formula IVe and Formula IVf in various molar ratios. For example, in some embodiments, single stranded RNA molecules according to Formula IVe and Formula IVf may be combined in about equimolar ratios. Some, none or all single-stranded RNA molecules according to Formula IVe and Formula IVf may hybridize with another complementary single-stranded RNA molecule to form double-stranded RNA molecules.

In other embodiments, single stranded RNA molecules according to Formula IVe may be combined in a composition with single stranded RNA molecules according to Formula IVf in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVe or Formula IVf may hybridize with another complementary single-stranded RNA molecule to form double-stranded RNA molecules.

In other embodiments, single stranded RNA molecules according to Formula IVf may be combined in a composition with single stranded RNA molecules according to Formula IVe in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVe or Formula IVf may hybridize with another complementary single-stranded RNA molecule to form double-stranded RNA molecules.

In some embodiments of the invention, compositions may comprise single-stranded RNA molecules according to Formula IVg or Formula IVh, or both Formula IVg and Formula IVh in various molar ratios. For example, in some embodiments, single stranded RNA molecules according to Formula IVg and Formula IVh may be combined in about equimolar ratios. Some, none or all single-stranded RNA molecules according to Formula IVg and Formula IVh may hybridize with another complementary single-stranded RNA molecule to form double-stranded RNA molecules.

In other embodiments, single stranded RNA molecules according to Formula IVg may be combined in a composition with single stranded RNA molecules according to Formula IVh in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVg or Formula IVh may hybridize with another complementary single-stranded RNA molecule to form double-stranded RNA molecules.

In other embodiments, single stranded RNA molecules according to Formula IVh may be combined in a composition with single stranded RNA molecules according to Formula IVg in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVg or Formula IVh may hybridize with another complementary single-stranded RNA molecule to form double-stranded RNA molecules.

In some embodiments of the invention, compositions may comprise single-stranded RNA molecules according to Formula IVi or Formula IVj, or both Formula IVi and Formula IVj in various molar ratios. For example, in some embodiments, single stranded RNA molecules according to Formula IVi and Formula IVj may be combined in about equimolar ratios. Some, none or all single-stranded RNA molecules according to Formula IVi and Formula IVj may hybridize with another complementary single-stranded RNA molecule to form double-stranded RNA molecules.

In other embodiments, single stranded RNA molecules according to Formula IVi may be combined in a composition with single stranded RNA molecules according to Formula IVj in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVi or Formula IVj may hybridize with another complementary single-stranded RNA molecule to form double-stranded RNA molecules.

In other embodiments, single stranded RNA molecules according to Formula IVj may be combined in a composition with single stranded RNA molecules according to Formula IVi in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula IVi or Formula IVj may hybridize with another complementary single-stranded RNA molecule to form double-stranded RNA molecules.

In some embodiments, pairs of single stranded nucleic acids, for example, Formulas IVe and IVf, or Formulas IVg and IVh, or Formulas IVi and IVj, may hybridize and/or concatemerize under some thermodynamic, ionic or pH conditions.

### Nucleic Acids Comprising CPG Motifs

Double-stranded nucleic acid molecule according to various embodiments of the invention that comprise a CpG motif, where the CpG motif comprises at least one LNA, are generally described by Formulas VIa-VId:

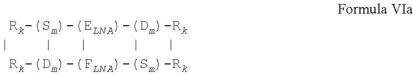

Formula VIa represents a double-stranded nucleic acid molecule having a first strand Rₖ—(Sₘ)-(E_{LNA})-(Dₘ)-Rₖ and a second strand Rₖ-(Dₘ)-(F_{LNA})—(Sₘ)—Rₖ, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

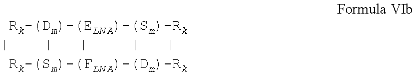

Formula VIb represents a double-stranded nucleic acid molecule having a first strand Rₖ-(Dₘ)-(E_{LNA})-(Sₘ)—Rₖ and a second strand Rₖ—(Sₘ)-(F_{LNA})-(Dₘ)-Rₖ, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

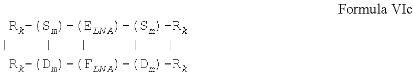

Formula VIc represents a double-stranded nucleic acid molecule having a first strand Rₖ—(Sₘ)-(E_{LNA})-(Sₘ)—Rₖ and a second strand Rₖ-(Dₘ)-(F_{LNA})-(Dₘ)-Rₖ, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

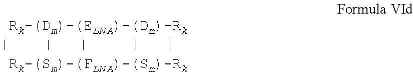

Formula VId represents a double-stranded nucleic acid molecule having a first strand Rₖ-(Dₘ)-(E_{LNA})-(Dₘ)-Rₖ and a second strand Rₖ—(Sₘ)—(F_{LNA})—(Sₘ)—Rₖ, with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

For each of Formula VIa-d;

E_{LNA} is CpG or a CpG motif, where one or more than one of the nucleosides, C, G, comprising the CpG or the CpG motif, is an LNA;

F_{LNA} is CpG or a CpG motif, where one or more than one of the nucleosides, C, G, comprising the CpG or the CpG motif, is an LNA;

m is as described for Formula IIa

R and k are as described for Formula IIb; and

S is inosine, an inosine-analogue nucleoside, adenine or an adenine-analogue nucleoside;

D is cytosine, a cytosine-analogue nucleoside, uracil, or a uracil-analogue nucleoside;

In some embodiments of the invention which are not to be considered limiting in any manner, the CpG motif may comprise two hexamer sequences of LNA nucleosides:

(SEQ ID NO: 23)

E_{LNA} = 5′-G_{LNA} T_{LNA} C_{LNA} G_{LNA} T_{LNA} T_{LNA}-3′;
and

(SEQ ID NO: 24)

F_{LNA} = 5′-A_{LNA} A_{LNA} C_{LNA}G_{LNA} A_{LNA} C_{LNA}-3′.

Non-limiting examples of such sequences are generally described by Formulas VIe to VIh:

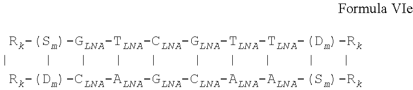

Formula VIe represents a double-stranded nucleic acid molecule having a first strand

VIi: Rₖ-(Sₘ)-G_{LNA}-T_{LNA}-C_{LNA}-G_{LNA}-T_{LNA}-T_{LNA}-(Dₘ)-Rₖ

and a second strand

VIj: Rₖ-(Dₘ)-C_{LNA}-A_{LNA}-G_{LNA}-C_{LNA}-A_{LNA}-A_{LNA}-(Sₘ)-
Rₖ,

with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

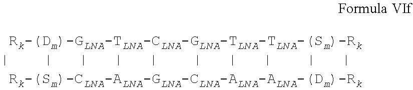

Formula VIf represents a double-stranded nucleic acid molecule having a first strand

VIk: Rₖ-(Dₘ)-G_{LNA}-T_{LNA}-C_{LNA}-G_{LNA}-T_{LNA}-T_{LNA}-(Sₘ)-Rₖ

and a second strand

VIl: Rₖ-(Sₘ)-C_{LNA}-A_{LNA}-G_{LNA}-C_{LNA}-A_{LNA}-A_{LNA}-(Dₘ)-
Rₖ,

with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

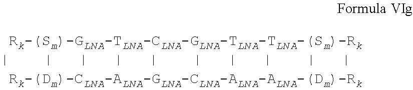

Formula VIg represents a double-stranded nucleic acid molecule having a first strand

VIm: Rₖ-(Sₘ)-G_{LNA}-T_{LNA}-C_{LNA}-G_{LNA}-T_{LNA}-T_{LNA}-(Sₘ)-Rₖ

and a second strand

VIn: Rₖ-(Dₘ)-C_{LNA}-A_{LNA}-G_{LNA}-C_{LNA}-A_{LNA}-A_{LNA}-(Dₘ)-
Rₖ,

with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

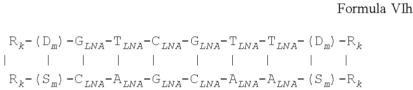

Formula VIh represents a double-stranded nucleic acid molecule having a first strand

VIo: Rₖ-(Dₘ)-G_{LNA}-T_{LNA}-C_{LNA}-G_{LNA}-T_{LNA}-T_{LNA}-(Dₘ)-Rₖ

and a second strand

VIp: Rₖ-(Sₘ)-C_{LNA}-A_{LNA}-G_{LNA}-C_{LNA}-A_{LNA}-A_{LNA}-(Sₘ)-
Rₖ,

with bonding between complimentary nucleosides represented by a single horizontal line. The first strand is represented in a 5′ to 3′ direction (left to right), while the second strand is represented in an anti-parallel orientation to the first strand (appearing as 3′-5′ when read left to right).

For each of Formulas VI e-VIh and VIi to VIp;

m is as described for Formula IIa

R and k are as described for Formula IIb; and

S is inosine, an inosine-analogue nucleoside, adenine or an adenine-analogue nucleoside;

D is cytosine, a cytosine-analogue nucleoside, uracil, or a uracil-analogue nucleoside;

### Concatemeric Combinations

IN some embodiments of the invention, the double-stranded nucleic acids comprising at least one CpG motif comprising at least one LNA nucleoside may include unpaired nucleosides, forming a ‘sticky end’ and may form concatemers. Formulae VIIa-VIIh (shown below in a 5′-3′ orientation, read left to right) represent single-stranded nucleic acids that hybridize according to sequence complementarity to form the double-stranded nucleic acids, for example as those described above in Formulas VIa to VIh. A double-stranded nucleic acid comprising a ‘sticky end’ may also be referred to as a monomer of a concatemeric polymer, according to some embodiments of the invention. Formula VIIa to VIIh are shown below followed by examples of combinations of nucleic acids comprising Formula VIIa to VIIh.

| | |
|---|---|
| Rₖ-(Sₘ)-(E_{LNA}) | Formula VIIa |
| Rₖ-(Dₘ)-(F_{LNA}) | Formula VIIb |
| Rₖ-(Dₘ)-(E_{LNA}) | Formula VIIc |
| Rk-(Sₘ)-(F_{LNA}) | Formula VIId |
| (E_{LNA})-(Sₘ)-Rₖ | Formula VIIe |
| (F_{LNA})-(Dₘ)-Rₖ | Formula VIIf |
| (E_{LNA})-(Dₘ)-Rₖ | Formula VIIg |
| (F_{LNA})-(Sₘ)-Rₖ | Formula VIIh |

For each of Formula VIIe-VIIh;

E_{LNA} is CpG or a CpG motif, where one or more than one of the nucleosides, C, G, comprising the CpG or the CpG motif is an LNA;

F_{LNA} is CpG or a CpG motif, where one or more than one of the nucleosides, C, G, comprising the CpG or the CpG motif is an LNA;

m is as described for Formula IIa

R and k are as described for Formula IIb; and

S is inosine, an inosine-analogue nucleoside, adenine or an adenine-analogue nucleoside;

D is cytosine, a cytosine-analogue nucleoside, uracil, or a uracil-analogue nucleoside;

In some embodiments of the invention, compositions may comprise single-stranded RNA molecules according to one or more than one nucleic acid of Formula VIIa to VIIh, or a combination of at least two or more than two nucleic acids of Formula VIIa to VIIh in various molar ratios. For example, in some embodiments, single stranded RNA molecules according to Formula VIIa and Formula VIIb may be combined in about equimolar ratios. Some, none or all single-stranded RNA molecules according to Formula VIIc, Formula VIId, Formula VIIe, Formula VIIf, Formula VIIg, or Formula VIIh may hybridize with another complementary single-stranded RNA molecule to form double-stranded RNA molecules.

In other embodiments, single stranded RNA molecules according to Formula VIIa may be combined in a composition with single stranded RNA molecules according to Formula VIIb in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIa or Formula VIIb may hybridize with another complementary single-stranded RNA molecule to form double-stranded RNA molecules.

In other embodiments, single stranded RNA molecules according to Formula VIIc may be combined in a composition with single stranded RNA molecules according to Formula VIId in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIc or Formula VIId may hybridize with another complementary single-stranded RNA molecule to form double-stranded RNA molecules.

In other embodiments, single stranded RNA molecules according to Formula VIIe may be combined in a composition with single stranded RNA molecules according to Formula VIIf in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIe or Formula VIIf may hybridize with another complementary single-stranded RNA molecule to form double-stranded RNA molecules.

In other embodiments, single stranded RNA molecules according to Formula VIIg may be combined in a composition with single stranded RNA molecules according to Formula VIIh in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIg or Formula VIIh may hybridize with another complementary single-stranded RNA molecule to form double-stranded RNA molecules.

In other embodiments, single stranded RNA molecules according to Formula VIIg may be combined in a composition with single stranded RNA molecules according to Formula VIId in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIg or Formula VIId may hybridize with another complementary single-stranded RNA molecule to form double-stranded RNA molecules.

In other embodiments, single stranded RNA molecules according to Formula VIIa may be combined in a composition with single stranded RNA molecules according to Formula VIIf in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIa or Formula VIIf may hybridize with another complementary single-stranded RNA molecule to form double-stranded RNA molecules.

In other embodiments, single stranded RNA molecules according to Formula VIIe may be combined in a composition with single stranded RNA molecules according to Formula VIIb in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIe or Formula VIIb may hybridize with another complementary single-stranded RNA molecule to form double-stranded RNA molecules.

In other embodiments, single stranded RNA molecules according to Formula VIIc may be combined in a composition with single stranded RNA molecules according to Formula VIIh in a molar excess of about 10%, 20%, 30%, 40%, 50%, 60%, 70%, 80%, 90% or in a fold excess of about 2, 3, 4, 5, 6, 7, 8, 9 or 10-fold. Some, none or all single-stranded RNA molecules according to Formula VIIc or Formula VIIh may hybridize with another complementary single-stranded RNA molecule to form double-stranded RNA molecules.

Exemplary base-pairing arrangements are illustrated below. Other pairings and arrangements of double-stranded nucleic acids according to various embodiments of the invention, will be apparent to those of skill in the art. For each exemplary pairing illustrated below, the first strand is provided in a 5′-3′ orientation, and the second strand is provided in a 3′-5′ orientation when read left to right, according to convention in the art.

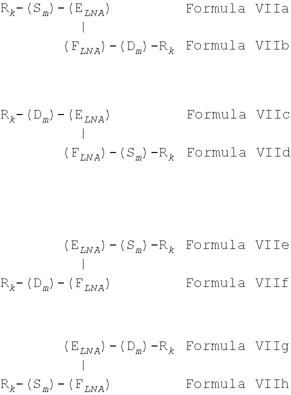

Alternate pairings for Formulae VIIa-VIIh, where k=0:

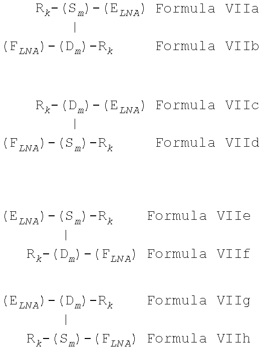

such monomers may concatenate to form a longer or circular double-stranded nucleic acid polymer.

In some embodiments of the invention, the single-stranded nucleic acid molecules according to formulae VIIa-h may base-pair to form blunt-ended double-stranded nucleic acid molecules. Exemplary base-pairing arrangements are illustrated below.

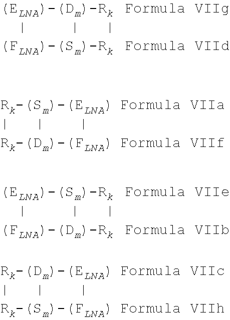

In the above example, k is an interger from 1 to 10 (and not zero), R may be any nucleoside or group of nucleosides as described above, wherein at least one nucleoside from each of the first and second strands form a hydrogen-bonded base pairing.

In some embodiments, pairs of single stranded nucleic acids, for example Formula VIIa and VIIb, or Formula VIIc and VIId, or Formula VIIe and VIIf or Formula VIIg and VIIh, or Formula VIIg and VIId, or Formula VIIa and VIIf, or Formula VIIe and VIIb, or Formula VIIc and VIIh, may concatemerize under some thermodynamic, ionic or pH conditions.

In some embodiments of the invention, nucleic acid compositions comprising polyinosine and polycytidine (e.g. “polyIC”) may comprise large molecular weight polymers. For example, a polyIC composition may comprise a molecular weight range from about 100 kDa to about 5000 kDa, or any molecular weight therebetween, for example 100, 500, 1000, 1500, 2000, 2500, 3000, 3500, 4000, 4500 or 5000 kDa, or any molecular weight therebetween; and may comprise single strands of polyI of about 150 to about 5000 residues (or any number of residues therebetween, for example 150, 200, 300, 400, 500, 1000, 1500, 2000, 2500, 3000, 3500, 4000, 4500 or 5000, or any number of residues therebetween) and single strands of polyC of about 150 to about 5000 residues (or any number of residues therebetween, for example 150, 200, 300, 400, 500, 1000, 1500, 2000, 2500, 3000, 3500, 4000, 4500 or 5000, or any number of residues therebetween) of varying lengths hybridized together. The resulting polyIC composition may comprise both single stranded and double-stranded regions.

Quantities and/or concentrations may be calculated on a mass/mass basis (e.g. micrograms or milligrams per kilogram of subject), or may be calculated on a mass/volume basis (e.g. concentration, micrograms or milligrams per milliliter). Using a mass/volume unit, an adjuvant may be present at an amount from about 0.1 ug/ml to about 20 mg/ml, or any amount therebetween, for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 ug/ml, or any amount therebetween; or from about 1 ug/ml to about 2000 ug/ml, or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, ug/ml or any amount therebetween; or from about 10 ug/ml to about 1000 ug/ml or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 ug/ml, or any amount therebetween; or from about 30 ug/ml to about 1000 ug/ml or any amount therebetween, for example 30.0, 35.0, 40.0, 50.0 60.0, 70.0, 80.0, 90.0, 100, 120, 140, 160 180, 200, 250, 500, 750, 1000 ug/ml.

An “effective amount” of an adjuvant as used herein refers to the amount of adjuvant required to have an immunostimulatory effect when co-administered with an immunogen wherein the immunogen demonstrates biological activity. The effective amount may be calculated on a mass/mass basis (e.g. micrograms or milligrams per kilogram of subject), or may be calculated on a mass/volume basis (e.g. concentration, micrograms or milligrams per milliliter). Using a mass/volume unit, an immunogen may be present at an amount from about 0.1 ug/ml to about 20 mg/ml, or any amount therebetween, for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 ug/ml, or any amount therebetween; or from about 1 ug/ml to about 2000 ug/ml, or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, ug/ml or any amount therebetween; or from about 10 ug/ml to about 1000 ug/ml or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 ug/ml, or any amount therebetween; or from about 30 ug/ml to about 1000 ug/ml or any amount therebetween, for example 30.0, 35.0, 40.0, 50.0 60.0, 70.0, 80.0, 90.0, 100, 120, 140, 160 180, 200, 250, 500, 750, 1000 ug/ml.

Compositions according to various embodiments of the invention, including adjuvant compositions, may be administered as a dose comprising an effective amount of an adjuvant. The dose may comprise from about 0.1 ug/kg to about 20 mg/kg (based on the mass of the subject), for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 ug/kg, or any amount therebetween; or from about 1 ug/kg to about 2000 ug/kg or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000 ug/kg, or any amount therebetween; or from about 1000 ug/kg to about 1000 ug/kg or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 ug/kg, or any amount therebetween; or from about 30 ug/kg to about 1000 ug/kg or any amount therebetween, for example 30.0, 35.0, 40.0, 50.0 60.0, 70.0, 80.0, 90.0, 100, 120, 140, 160 180, 200, 250, 500, 750, 1000 ug/kg.

One of skill in the art will be readily able to interconvert the units as necessary, given the mass of the subject, the concentration of the adjuvant composition, individual components or combinations thereof, or volume of the adjuvant composition, individual components or combinations thereof, into a format suitable for the desired application.

The amount of a composition administered, where it is administered, the method of administration and the timeframe over which it is administered may all contribute to the observed effect. As an example, a composition may be administered systemically e.g. intravenous administration and have a toxic or undesirable effect, while the same composition administered subcutaneously may not yield the same undesirable effect. In some embodiments, localized stimulation of immune cells in the lymph nodes close to the site of subcutaneous injection may be advantageous, while a systemic immune stimulation may not. Alternately, a lesser total amount, or an amount of a composition comprising different mass or molar ratios of polyIC to cationic polymer, or differing molecular weight cutoffs of the polyIC or cationic polymer may be useful as an immunostimulant, without exhibiting significant therapeutic activity in the absence of an immunogen.

Adjuvants according to various embodiments of the invention may be formulated with any of a variety of pharmaceutically acceptable excipients, frequently in an aqueous vehicle such as Water for Injection, Ringer's lactate, isotonic saline or the like. Pharmaceutically acceptable excipients may include, for example, salts, buffers, antioxidants, complexing agents, tonicity agents, cryoprotectants, lyoprotectants, suspending agents, emulsifying agents, antimicrobial agents, preservatives, chelating agents, binding agents, surfactants, wetting agents, anti-adherents agents, disentegrants, coatings, glidants, deflocculating agents, anti-nucleating agents, surfactants, stabilizing agents, non-aqueous vehicles such as fixed oils, polymers or encapsulants for sustained or controlled release, ointment bases, fatty acids, cream bases, emollients, emulsifirers, thickeners, preservatives, solubilizing agents, humectants, water, alcohols or the like. See, for example, Berge et al. (1977. J. Pharm Sci. 66:1-19), or Remington—The Science and Practice of Pharmacy, 21^{st} edition. Gennaro et al editors. Lippincott Williams & Wilkins Philadelphia (both of which are herein incorporated by reference).

In some embodiments, the adjuvant composition may comprise carboxymethylcellulose (CMC) or a polycationic polymer, or a combination thereof. A “polycationic polymer” may alternately be referred to as a “cationic polymer” without exclusion or limitation. Examples of polycationic polymers include but are not limited to poly-lysine, polyornithine, polyarginine, or a polypeptide comprising a majority of cationic amino acids. Examples of cationic amino acids include arginine, ornithine, lysine, histidine, 5-hydroxylysine, 6-N-methyllysine, (6-N-6-N) dimethyllysine, (6-N, 6-N, 6-N) trimethyllysine, or the like. Amino acid polymers may comprise all -L, all -D or a mixture of L and D isomers. Molecular weight, concentrations and methods of preparation of a poly-L-lysine polycationic polymer may be found in, for example, U.S. Pat. No. 4,349,538 (which is incorporated herein by reference).

Without wishing to be bound by theory, inclusion of carboxymethylcellulose in an adjuvant composition in combination with an antigen may enhance a ‘depot’ effect when the adjuvant and antigen combination is administered to a subject, delaying dispersion of the antigen within the subject. Again, without wishing to be bound by theory, inclusion of carboxymethylcellulose in an adjuvant composition, in combination with an antigen, may protect other components of the adjuvant and/or the antigen from degradation when administered to the subject.

Without wishing to be bound by theory, smaller cationic peptides are known generally to facilitate the entry of proteins or other macromolecules into cells (protein translocation—see, for example, Dietz et al. 2003. Mol Cell Neurosci 27:85-131). An example of a smaller cationic peptide is a polymer having an average of 60 degrees of polymerization (about 60 residues). Egyed (U.S. Pat. No. 7,148,191) discloses the use of a polyarginine polymer having an average of 60 degrees of polymerization (about 10,000 Da).

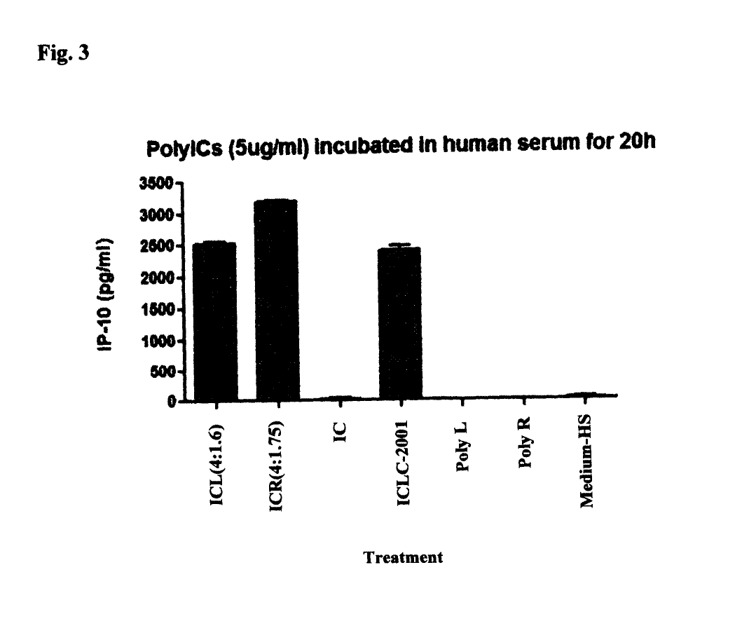

Further, without wishing to be bound by theory, longer polyarginine, polylysine or polyornithine compositions, for example those comprising 50 residues to about 700 residues, or any amount therebetween, for example 50, 75, 100, 125, 150, 175, 200, 225, 250, 275, 300, 325, 350, 375, 400, 425, 450, 475, 500, 525, 550, 575, 600, 625, 650, 675 or 700 residues, or any amount therebetween, such as those described herein, may provide a protective effect for the polyIC compositions, by protecting them from degradation (see, for example, the data presented in FIG. 3 and the associated text of the specification). Longer polyarginine, polylysine or polyornithine compositions may, in addition to the protective effect, facilitate the entry of macromolecules into a cell.

Molecular weight of polycationic polymers, such as those comprising amino acids may be determined as an average range based on viscosity studies; the degree of crosslinking may be determined by multi-angle laser light scattering (MALLS). MALLS may also be used in combination with size-exclusion chromatography (SEC) to obtain molecular weight profiles of polycationic polymers, according to some embodiments of the invention.

The synthesis of polyIC nucleic acid polymers and polycationic polymers according to some embodiments of the invention may lead to a population of molecules having a range of molecular weights, such that the population is described as having an average MW of, for example greater than a particular quantity, or in a range of about a first value to about a second value, or less than a particular quantity. In such populations, determination of the exact molar ratios of the individual polymer molecules may not be possible, and the ratios may be described as mass ratios—e.g. 4:1.75 ug or 25 ug/ml, and the volume used provided in a protocol for the experiment. The molar ratios of the individual monomers may be determined from this information, using standard calculations known to those skilled in the art. For example, the MW of arginine is 174.2 g/mol; the MW of ornithine is 132.1 g/mol; the MW of lysine is 146.18 g/mol; the MW of inosine monophosphate is about 348.2 g/mol; the MW of cytidine monophosphate is about 323.2 g/mol.

When referring to a polymer of amino acids, the term “degree of polymerization” or “average degree of polymerization”, both abbreviated as ‘dp’, may also be used. The average degree of polymerization may be obtained by dividing the average calculated MW of the polymer by the MW of the monomer.

In other embodiments, the polyarginine, polyornithine or polylysine polymer may comprise from about 50 to about 700 residues or more, or any amount therebetween, for example 50, 75, 100, 125, 150, 175, 200, 225, 250, 275, 300, 325, 350, 375, 400, 425, 450, 475, 500, 525, 550, 575, 600, 625, 650, 675 or 700 residues, or any amount therebetween; or from about 100 residues to about 600 residues or any amount therebetween, for example 100, 125, 150, 175, 200, 225, 250, 275, 300, 325, 350, 375, 400, 425, 450, 475, 500, 525, 550, 575 or 600 residues, or any amount therebetween; or from about 200 to about 600 residues or any amount therebetween, for example 200, 225, 250, 275, 300, 325, 350, 375, 400, 425, 450, 475, 500, 525, 550, 575 or 600 residues, or any amount therebetween; or from about 300 to about 500 residues or any amount therebetween, for example 300, 325, 350, 375, 400, 425, 450, 475 or 500 residues, or any amount therebetween. In other embodiments, the polyarginine may have a molecular weight, or an average molecular weight of, for example, about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124 or 125 kDa, or any amount therebetween.

In other embodiments, polyarginine, polyornithine, polylysine polymers, or a combination of polyarginine, polyornithine, polylysine polymers according to the sizes or masses described above may be combined in a variety of mass ratios to provide a combination polymer. For example, polyarginine and polyornithine may be combined in a mass ratio from about 100:1 to about 1:100, or any amount therebetween. For example, 99:1 to about 1:99, from about 98:2 to about 2:98, from about 97:3 to about 3:97, from about 96:4 to about 4:96, or 95:5 to about 5:95, or any amount therebetween. In another example, polyarginine and polylysine may be combined in a mass ratio from about 100:1 to about 1:100, or any amount therebetween. For example, 99:1 to about 1:99, from about 98:2 to about 2:98, from about 97:3 to about 3:97, from about 96:4 to about 4:96, or 95:5 to about 5:95, or any amount therebetween. In another example, polyornithine and polylysine may be combined in a mass ratio from about 100:1 to about 1:100, or any amount therebetween. For example, 99:1 to about 1:99, from about 98:2 to about 2:98, from about 97:3 to about 3:97, from about 96:4 to about 4:96, or 95:5 to about 5:95, or any amount therebetween. Additionally, other combinations of polyarginine, polyornithine, and polylysine polymers may be used as desired. Any compound or composition administered to a subject, whether human or non-human, will be exposed to the nucleases, proteases and other enzymes that are found in the subject, and degraded accordingly. Mode of administration may also influence the degradation rate of the compound or composition. For example, administering a compound or composition intravenously exposes the compound or composition to nucleases present in the serum, while a subcutaneous administration route reduces the exposure to serum nucleases.

In addition to differences in enzyme exposure due to administration route, the nature of the subject may also provide for differences in exposure to proteases, nucleases and other degradative enzymes. A variety of species may be used as test subjects to investigate the efficacy of an adjuvant composition. Studies have previously demonstrated that significant differences are observed with respect to the serum RNAse complement found in rabbit, human, mouse, dog, guinea pig, horse, monkey, chicken and fetal calf sera (Nordlund et al 1970. Proc Soc Exp Biol Med 133:439-444). A pyrogenic nucleic acid composition, poly I:C, was tested for the ability to induce fever in a rabbit model. Mouse, rabbit, dog, guinea pig, horse and monkey serum demonstrated limited to no inactivation of polyIC; chicken and fetal calf inactivated the pyrogen to a moderate extent, while human serum destroyed the pyrogenicity of the polyIC. Without wishing to be bound by theory, a significantly greater dose of polyIC may be required to be administered to a human subject (compared to, for example, a mouse) to achieve a therapeutically effect, to overcome the greater degradative characteristics of the human serum. Depending on the nature of the polyIC, such a ‘therapeutically effective’ dose may be prohibitively toxic. Conversely, a ‘therapeutically effective’ dose in a mouse may comprise significantly less material, as the rate of degradation is not as significant.

Without wishing to be bound by theory, combining polyIC with a cationic polymer, may be protective or stabilizing of the polyIC when administered to a subject and thus exposed to nucleases or other serum components that may degrade the polyIC. This protective effect may allow for reduction of the quantity of polyIC required to achieve a ‘therapeutically effective’ dose.

Therefore, the invention provides for a method of stabilizing a polyIC complex comprising combining polyIC and a cationic polymer, the cationic polymer comprising from about 100 to about 700 amino acid residues and the mass:mass ratio of polyIC:cationic polyumer is from about 4:14 to about 4:3 mg/ml, or from about 4.15 to about 4:2 mg/ml.

In some embodiments of the invention, polyIC is combined with a polycationic polymer in a mass ratio of about 4:1.4 (mg of polyIC:mg of polycationic polymer) to about 4:3, or any amount therebetween, for example, 4:1.45, 4:1.5, 4:1.55, 4:1.6, 4:1.65, 4:1.7, 4:1.75, 4:1.8, 4:1.85, 4:1.9, 4:2, 4:2.1, 4:2.2, 4:2.3, 4:2.4, 4:2.5, 4:2.6, 4:2.7, 4:2.8, 4:2.9 or any amount therebetween.

It has been observed that premixing the polycationic amino acid polymer with the nucleic acid polymer results in precipitation at some ratios above 4:3 (mg polyIC:mg polycationic polymer).

Therefore, the invention provides for an adjuvant composition comprising polyIC and a cationic polymer, the cationic polymer comprising from about 100 to about 700 amino acid residues, and the mass:mass ration of the polyIC:cationic polymer is from about 4:1.4 to about 4:3, or from about 4:1.5 to about 4:2 mg/ml. The polycationic polymer may be polyarginine.

U.S. Pat. No. 7,148,191 to Egyed describes mass:mass ratios of 0.5:1, 1:1 and 3:1 (mg polyIC:mg polyarginine). The polyarginine polymer having an average degree of polymerization of 60 arginine residues. Egyed discloses that a polyarginine having a degree of polymerization of about 60 residues, does not affect polyIC induced in vitro maturation of dendritic cells (DCs).

According to some embodiments of the invention, and without wishing to be bound by theory, compositions comprising polyIC and polyarginine having a mass:mass ratio of about 4:1.5 to about 4:2, according to some embodiments of the invention, do enhance maturation of DCs in vitro. Exemplary results are shown in Table 1. Immature DCs are ‘stimulated’, ‘induced’ or ‘activated’ to become mature DCs upon stimulation of, for example select TLRs, such as TLR3 or TLR8. DC maturation may be identified by upregulation of some cell-surface receptors, for example, CD80, CD86, CD40, MHC I, MHC II or others. Mature DCs act as antigen presenting cells, and may assist in activation of various T cells and/or B cells, enhancing the immune response to an antigen. Of particular note is the change in CD86 expression (Table 1, Example 11 herein) when cells are exposed to polyIC:polyarginine compositions according to some embodiments of the invention, compared to the lack of change in CD86 expression as reported by Egyed. Clearly, the compositions used differ.

Therefore, the invention provides for a method of inducing dendritic cell maturation comprising administering a composition comprising polyIC and a cationic polymer, the cationic polymer comprising from about 100 to about 700 amino acid residues and the mass: mass ration of polyIC:cationic polymer is from about 4:14 to about 4:3 mg/ml, or from about 4.15 to about 4:2 mg/ml. The polycationic polymer may be polyarginine.

Compositions comprising an adjuvant according to various embodiments of the invention may be administered by any of several routes, including, for example, subcutaneous injection, intraperitoneal injection, intramuscular injection, intravenous injection, epidermal or transdermal administration, mucosal membrane administration, orally, nasally, rectally, topically or vaginally. Alternately, such compositions may be directly injected into a tumor, or a lymph node near a tumor, or into an organ or tissue near a tumor, or an organ or tissue comprising tumor cells. See, for example, Remington—The Science and Practice of Pharmacy, 21^{st} edition. Gennaro et al editors. Lippincott Williams & Wilkins Philadelphia. Carrier formulations may be selected or modified according to the route of administration.

Compositions according to various embodiments of the invention may be applied to epithelial surfaces. Some epithelial surfaces may comprise a mucosal membrane, for example buccal, gingival, nasal, tracheal, bronchial, gastrointestinal, rectal, urethral, vaginal, cervical, uterine and the like. Some epithelial surfaces may comprise keratinized cells, for example, skin, tongue, gingival, palate, anus or the like. Compositions according to various embodiments of the invention may be provided in a unit dosage form, or in a bulk form suitable for formulation or dilution at the point of use.

Compositions according to various embodiments of the invention may be administered to a subject in a single-dose, or in several doses administered over time. Dosage schedules may be dependent on, for example, the subject's condition, age, gender, weight, route of administration, formulation, or general health. Dosage schedules may be calculated from measurements of adsorption, distribution, metabolism, excretion and toxicity in a subject, or may be extrapolated from measurements on an experimental animal, such as a rat or mouse, for use in a human subject. Optimization of dosage and treatment regimens are discussed in, for example, Goodman & Gilman's The Pharmacological Basis of Therapeutics 11^{th} edition. 2006. LL Brunton, editor. McGraw-Hill, New York, or Remington—The Science and Practice of Pharmacy, 21^{st} edition. Gennaro et al editors. Lippincott Williams & Wilkins Philadelphia.

Adjuvant compositions according to various embodiments of the invention may be combined with a second adjuvant composition and used or administered as described. Examples of such second adjuvant compositions include, but are not limited to, adjuvants or their components e.g. polyIC, polyICLC, polyIC/R, aluminium hydroxide, alum, Alhydrogel™ (aluminum trihydrate) or other aluminium-comprising salts, virosomes, nucleic acids comprising CpG motifs, squalene, oils, MF59, QS21, various saponins, virus-like particles, monophosphoryl-lipidA/trehalose dicorynomycolate, toll-like receptor agonists, copolymers such as polyoxypropylene and polyoxyethylene, or the like.

Compositions for topical, epidermal, transdermal or mucosal membrane applications (generally referred to herein as ‘topical preparations’) may be prepared as a cream, ointment, oil, gel, paste, powder, lotion, liniment, emulsion or the like, and may be generally referred to as a “topical base”. The amount of immune response modifier may be present in an amount effective to stimulate a localized immune response (for example, localized to the general area where the preparation is applied), or a systemic immune response, or a localized and systemic immune response. For example, the immune response modifier may be present in an amount from about 0.01% to about 20% by weight, based on the total weight of the preparation, or any amount therebetween; for example 0.01, 0.05, 1, 2, 3, 4, 5, 6, 7, 8, 9, 10, 11, 12, 13, 14, 15, 16, 17, 18, 19 or 20% by weight.

Topical preparations may further comprise lipids such as oils, or fatty acids (e.g. stearic acids, oleic acids or the like, or a combination thereof), emollients, emulsifiers, thickeners, preservatives or other excipients such as are known in the art.

For example, a topical preparation according to some embodiments of the invention may comprise one, or more than one of the following: polyIC and a cationic polymer, and a topical base; the cationic polymer comprising from about 100 to about 700 amino acid residues, wherein the mass:mass ratio of the polyIC:cationic polymer is from about 4:1.4 to about 4:3; and wherein the topical base may be one or more than one of a cream, ointment, oil, gel, paste, powder, lotion, liniment, emulsion, or fatty acid, emollient, emulsifier, thickener, preservative.

The present invention also provides for a method of inducing a localized immune response at or near an epithelial surface comprising applying a composition comprising PolyIC and a cationic polymer, and a topical base to an epithelial surface of a subject, the cationic polymer comprising from about 100 to about 700 amino acid residues, wherein the mass:mass ratio of the polyIC:cationic polymer is from about 4:1.5 to about 4:3

Compositions for topical, epidermal, transdermal or mucosal membrane applications may be included as a coating on, or embedded in, transdermal patch, a bioadhesive material or an adhesive composition, for example a pressure-sensitive adhesive composition. Examples of bioadhesive materials are disclosed in, for example, U.S. Pat. No. 6,562,363. Examples of adhesive compositions are disclosed in, for example, U.S. Pat. No. 5,238,944. Excipients suitable for topical compositions and methods of formulating topical compositions are well-known in the art, and are described in, for example Goodman & Gilman's The Pharmacological Basis of Therapeutics (supra), or Remington (supra).

In the context of the present invention, the terms “treatment,”, “treating”, “therapeutic use,” or “treatment regimen” as used herein may be used interchangeably are meant to encompass prophylactic, palliative, and therapeutic modalities of administration of the compositions of the present invention, and include any and all uses of the presently claimed compounds that remedy a disease state, condition, symptom, sign, or disorder caused by an inflammation-based pathology, cancer, infectious disease, allergic response, hyperimmune response, or other disease or disorder to be treated, or which prevents, hinders, retards, or reverses the progression of symptoms, signs, conditions, or disorders associated therewith. Thus, any prevention, amelioration, alleviation, reversal, or complete elimination of an undesirable disease state, symptom, condition, sign, or disorder associated with an inflammation-based pathology, or other disease or disorder that benefits from stimulation of the body's immune response, is encompassed by the present invention. A treatment may comprise administration of an effective amount of a composition as described herein, alone or in combination with an immunogen.

Efficacy of an adjuvant composition with or without immunogen may be demonstrated, for example, using a murine tumor model, such as the TC-1K model as described herein for the HPV-16 E7 immunogen. Establishment of tumors, followed by administration of the adjuvant composition to be tested, and subsequent monitoring of the tumor load increase or decrease provides an in vivo indicator of the ability of the adjuvant composition to stimulate the necessary immune response. For a model of prophylaxis, the adjuvant composition may be first administered to a naïve mouse, followed by administration of a dose of TC-1K cells sufficient to establish a tumor load in the animal (this dose size may vary, and may be empirically determined for each model system; alternately a dose of about 1×10⁵ to about 5×10⁵, or about 1×10⁶, or more, may be used).

A prophylactic dose of an adjuvant composition is a dose sufficient to provide a reduction in tumor load, relative to a control. A prophylactic dose may also be referred to as a preventative dose.

Compositions according to various embodiments of the invention may further comprise at least one immunogen, for example a viral or bacterial (“pathogen”) immunogen. An immunogen may be prepared from a killed whole-organism (a ‘killed vaccine’) or may be prepared from a specific protein, peptide or other substructure of the pathogen. Alternatively, the immunogen may be a fusion protein comprising a whole or partial protein or peptide from a pathogen, fused with another non-pathogen protein or peptide, such as a ‘His-Tag” or other moiety useful in purification of the immunogen. An immunogen may alternately be referred to as an ‘antigen’. An immunogen may be soluble in an aqueous medium, or a lipophilic medium (e.g. an oil, fat or cream) or may comprise a suspension in an aqueous or lipophilic medium. Specific proteins or peptides may be produced using molecular biology techniques or methods (“recombinant” proteins or peptides). Conventional techniques or methods used in recombinant molecular biology are described in, for example, Molecular Cloning: a Laboratory Manual 3^{rd} edition. Sambrook and Russell. CSHL Press, Cold Spring Harbour, N.Y.; Current Protocols in Molecular Biology, 2007 Ausubel et al editors. Wiley InterScience, New York; Current Protocols in Immunology, 2006 Coligan et al editors. Wiley InterScience, New York.

The immunogen may be a killed whole-organism, a protein, a peptide, a fusion protein, a fusion peptide, a recombinant protein, or a recombinant peptide, an amino acid sequence comprising a heat shock protein, an antigen from a bacterial fungal or viral pathogen, or a heat shock fusion protein comprising an antigen from a bacterial, fungal or viral pathogen, or one or more than one fragment or portion thereof. For example, the immunogen may comprose a human papillomavirus (HPV) immunogen such as a protein from HPV, or a fragment of a protein from HPV. Examples of such proteins include viral capsid proteins L1 and L2, non-structural proteins, E1, E2, E4, E5, or oncoproteins E6 or E7. The HPV immunogen may be fused with another protein, or fragment of another protein, such as a stress protein. Examples of stress proteins include heat shock proteins or other families of stress proteins including Lon, TF55, FKBPs, cyclophilins, ClpP, GrpE, ubiquitin, calnexin, protein disulfide isomerates and the like, see for example Macario, A. J. L., Cold Spring Harbor Laboratory Res. 25:59-70. 1995; Parsell, D. A. & Lindquist, S. Ann. Rev. Genet. 27:437-496 (1993); U.S. Pat. No. 5,232,833 (Sanders et al.) herein incorporated by reference. Examples of heat shock proteins include hsp60, hsp65 and hsp70, and an example of an immunogen according to some embodiments of the invention comprises HspE7. The HspE7 immunogen is described generally in WO 99/07860; and in a highly purified form in WO 2007/137427; both of which are herein incorporated by reference. In another example, the immunogen may be provided in the form of a nucleic acid sequence such as a DNA vaccine or viral expression vector so that the peptide or protein sequence comprising the immunogen is expressed in vivo. Various delivery systems, for example liposomes, are known in the art and will be suitable for such administration. For example, a site of administration may be ‘primed’ with an adjuvant composition according to various embodiments of the invention, followed by administration of the nucleic acid sequence. The adjuvant composition may comprise an antigen, or may lack a specific antigen. Other excipients that stabilize or otherwise enhance the immunostimulatory effect of the adjuvant and/or antigen may also be included in the adjuvant composition.

Examples of immunogens include, but are not limited to a protein, a peptide, a fusion protein, a fusion peptide, a recombinant protein or recombinant peptide or an amino acid sequence comprising a heat shock protein, an antigen from bacterial, fungal or viral pathogens, or heat shock fusion proteins (e.g. HspE7—WO 99/07860, U.S. Pat. No. 7,157,089) comprising antigens from bacterial, fungal or viral pathogens; or one or more than one fragment or portion thereof. Examples of bacterial, fungal or viral pathogens, include, but are not limited to, causative agents of the following diseases or disorders: papilloma, genital warts, influenza, hepatitis A, hepatitis B, hepatitis C, hepatitis D, hepatitis E, hepatitis G, Cytomegalovirus, Epstein Barr virus, AIDS, AIDS Related Complex, Chickenpox (Varicella), tooth decay (e.g. Streptococcus mutans), Common cold, Cytomegalovirus Infection, Colorado tick fever, Dengue fever, Ebola haemorrhagic fever, Hand, foot and mouth disease, Hepatitis, Herpes simplex, Herpes zoster, human papillomavirus (HPV), Influenza (Flu), Lassa fever, Measles, Marburg haemorrhagic fever, Infectious mononucleosis, Mumps, Poliomyelitis, Progressive multifocal leukencephalopathy, Rabies, Rubella, SARS, Smallpox (Variola), Viral encephalitis, Viral gastroenteritis (e.g. Norwalk virus, Rotavirus and the like), Viral meningitis, fifth disease, Viral pneumonia, West Nile disease, Yellow fever, Anthrax, Bacterial Meningitis, Botulism, Brucellosis, Campylobacteriosis, Cat Scratch Disease, Cholera, Diphtheria, Epidemic Typhus, Gonorrhea, Impetigo, Legionellosis, Leprosy (Hansen's Disease), Leptospirosis, Listeriosis, Lyme Disease, Melioidosis, MRSA infection, Nocardiosis, Pertussis (Whooping Cough), Plague, bacterial pneumonia, fungal pneumonia, Pneumococcal pneumonia, Psittacosis, Q fever, Roseola, Rocky Mountain Spotted Fever (RMSF), Salmonellosis, Scarlet Fever, Shigellosis, Syphilis,Tetanus, Trachoma, Tuberculosis, Tularemia, Typhoid Fever, Typhus, urinary tract infections, aspergillosis, basidiobolomycosis, candidiasis, cryptococcosis, coccidioidomycosis, dermatophytosis, ringworm, histoplasmosis, fungemia, paracoccidioidomycosis, pneumocystis pneumonia, and the like. Recombinant immunogens may be expressed using a recombinant expression system, for example bacterial, yeast, baculoviral, mammalian cell or plant expression system.

The invention, therefore, provides for a composition comprising polyIC and a cationic polymer; the cationic polymer comprising from about 100 to about 700 amino acid residues, wherein the mass:mass ratio of the polyIC:cationic polymer is from about 4:1.4 to about 4:3; and may further comprise an immunogen, or a second adjuvant composition, or both an immunogen and a second adjuvant composition. The polycationic polymer may be polyarginine. In some embodiments, the inclusion of a second adjuvant composition may provide a synergistic effect with respect to antibody production or inducing B lymphocytes (directly or indirectly, via another immune stimulating cell). In some embodiments, the second adjuvant composition may comprise an aluminum salt, such as alum.

A “full-length” protein, fusion protein or polypeptide, etc. includes a polypeptide comprising all, or most of the amino acid complement of a particular protein or polypeptide. For example, a few amino acids from the C and/or N terminus may be absent relative to the protein or polypeptide, but identifying domains, functional sequence, amino acids present in an active site or binding site of the immunogen, and sufficient amino acid sequence to specifically identify the protein are present in the “full-length” protein or polypeptide. Alternately, a “full-length” protein, fusion protein or polypeptide, etc. includes a polypeptide comprising one or more than one additional amino acids added to the C and/or N terminus, relative to the protein or polypeptide. These additional one or more than one amino acids may comprise, for example, a cysteine to allow for fusion, coupling or other linkages to another peptide or a surface, or a “His-tag” sequence (4-10 histidine residues) for isolation of the protein or polypeptide. A cleavage sequence to separate domains of the protein, or to allow for post-isolation removal of the His-tag may also be included.

A fragment or portion of a protein, fusion protein or polypeptide, etc. includes a peptide or polypeptide comprising a subset of the amino acid complement of a particular protein or polypeptide. The fragment may, for example, comprise an antigenic region, a stress-response-inducing region, or a region comprising a functional domain of the protein or polypeptide, etc. In some embodiments of the invention, the fragment may comprise a region or domain common to proteins of the same general family e.g. in some embodiments of the invention, the fragment may include sufficient amino acid sequence to specifically identify the full-length protein from which it is derived.

A protein or polypeptide, or fragment or portion of a protein or polypeptide may range in size from as small as 4-6 amino acids to the “full-length” of the protein or polypeptide. For example, a fragment or portion may be from about 1% to about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90% or from about 90% to about 100% of the full-length protein or polypeptide. Alternately, a fragment or portion may be from about 4 to about 10, or any amount therebetween, from 10 to about 50, or any amount therebetween, from about 50 to about 100 or any amount therebetween, from about 100 to about 150, or any amount therebetween, from about 150 to about 250 or any amount therebetween, from about 250 to about 500 or any amount therebetween. Alternately, a fragment or portion may be 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids long.

A protein or polypeptide, or fragment or portion of a protein or polypeptide is specifically identified when its sequence may be differentiated from others found in the same phylogenetic Species, Genus, Family or Order. Such differentiation may be identified by comparison of sequences. Comparisons of a sequence or sequences may be done using a BLAST algorithm (Altschul et al. 1009. J. Mol. Biol 215:403-410). A BLAST search allows for comparison of a query sequence with a specific sequence or group of sequences, or with a larger library or database (e.g. GenBank or GenPept) of sequences, and identify not only sequences that exhibit 100% identity, but also those with lesser degrees of identity.

Therefore, the invention provides for a composition comprising polyIC and a cationic polymer and an immunogen, the cationic polymer comprising from about 100 to about 700 amino acid residues and the mass: mass ration of polyIC:cationic polyumer is from about 4:14 to about 4:3 mg/ml, or from about 4.15 to about 4:2 mg/ml. The polycationic polymer may be polyarginine. The immunogen may be present in the composition at an amount from about 0.1 ug to about 20 mg.

In some embodiments of the invention, an immunogen may be a tumor or tumor cell antigen, a tumor-derived or tumor-cell derived antigen, or an antigen found in association with a cancer.

The term “cancer” has many definitions. According to the American Cancer Society, cancer is a group of diseases characterized by uncontrolled growth (and sometimes spread) of abnormal cells. Although often referred to as a single condition, it actually consists of more than 200 different diseases. Cancerous growths can kill when such cells prevent normal function of vital organs, or spread throughout the body, damaging essential systems. The composition of the present invention may be used to treat susceptible neoplasms in an animal or subject in a method that comprises administering to the animal or subject in need thereof an effective amount of a compound or composition of the present invention.

Non-limiting examples of different types of cancers against which compounds of the present invention may be effective as therapeutic agents include: carcinomas, such as neoplasms of the central nervous system, including glioblastoma multiforme, astrocytoma, oligodendroglial tumors, ependymal and choroid plexus tumors, pineal tumors, neuronal tumors, medulloblastoma, schwannoma, meningioma, and meningeal sarcoma; neoplasms of the eye, including basal cell carcinoma, squamous cell carcinoma, melanoma, rhabdomyosarcoma, and retinoblastoma; neoplasms of the endocrine glands, including pituitary neoplasms, neoplasms of the thyroid, neoplasms of the adrenal cortex, neoplasms of the neuroendocrine system, neoplasms of the gastroenteropancreatic endocrine system, and neoplasms of the gonads; neoplasms of the head and neck, including head and neck cancer, neoplasms of the oral cavity, pharynx, and larynx, and odontogenic tumors; neoplasms of the thorax, including large cell lung carcinoma, small cell lung carcinoma, non-small cell lung carcinoma, malignant mesothelioma, thymomas, and primary germ cell tumors of the thorax; neoplasms of the alimentary canal, including neoplasms of the esophagus, stomach, liver, gallbladder, the exocrine pancreas, the small intestine, veriform appendix, and peritoneum, adneocarcinoma of the colon and rectum, and neoplasms of the anus; neoplasms of the genitourinary tract, including renal cell carcinoma, neoplasms of the renal pelvis, ureter, bladder, urethra, prostate, penis, testis; and female reproductive organs, including neoplasms of the vulva and vagina, cervix, adenocarcinoma of the uterine corpus, ovarian cancer, gynecologic sarcomas, and neoplasms of the breast; neoplasms of the skin, including basal cell carcinoma, squamous cell carcinoma, dermatofibrosarcoma, Merkel cell tumor, and malignant melanoma; neoplasms of the bone and soft tissue, including osteogenic sarcoma, malignant fibrous histiocytoma, chondrosarcoma, Ewing's sarcoma, primitive neuroectodermal tumor, and angiosarcoma; neoplasms of the hematopoietic system, including myelodysplastic sydromes, acute myeloid leukemia, chronic myeloid leukemia, acute lymphocytic leukemia, HTLV-1 and T-cell leukemia/lymphoma, chronic lymphocytic leukemia, hairy cell leukemia, Hodgkin's disease, non-Hodgkin's lymphomas, and mast cell leukemia; and neoplasms of children, including acute lymphoblastic leukemia, acute myelocytic leukemias, neuroblastoma, bone tumors, rhabdomyosarcoma, lymphomas, renal tumors, and the like.

Compositions according to various embodiments of the present invention may be useful for treating diseases or disorders that involve epithelial surfaces. Examples of such diseases or disorders include, for example, cericial intraepithelial neoplasia, human papillomavirus, herpesvirus infection (e.g. type 1 or type 2), nodular basal cell carcinoma, cervical dysplasia, cervical cancer, warts, granuloma annulare, genital warts, actinic keratosis, basal cell nevus, xeroderma pigmentosrum, molluscum contagiosum, skin cancer, melanoma, vaginal lesions, epithelial dysplasias, bladder cancer, squamous cell carcinoma, vulvar intraepithelial neoplasia, vaginal intraepithelial neoplasia, psoriasis, eczema, condylomata acuminate, Bowen's disease, lentigo maligna, extramammary Paget's disease, bowenoid papulosis or the like.

In some embodiment of the invention, compositions comprising an immune response modifier may be used to incite or stimulate an immune response generally localized to one or a few site on a subject's body. Such compositions may be assayed for their ability to penetrate epithelial tissues, for example skin. Animal studies may be employed for this purpose. A composition is applied to an epithelial surface (e.g. skin) and at various times following the application, the animal's blood, serum or other body tissue is sampled for either the presence of one or more components of the composition. As another example, skin penetration may be assess using an in vitro method, for example the method described in U.S. Pat. No. 5,238,944.

Compositions may also be evaluated for their ability to reduce inflammation, lesion formation, viral load or local immune stimulation using in vivo methods. Generally, the composition is applied to one or more epithelial surfaces of a subject, and the localized area, blood, serum or body fluid sampled and/or assessed for the presence of one or more components of the composition, or for the presence of the subject immune response. Tissue samples may be obtained, for example, by biopsy, tissue scraping, cell wash, skin punch biopsy or the like; blood, serum or body fluid may be obtained by standard methods (e.g. drawing blood, separating blood cells from the blood to obtain plasma or serum, urine collection or the like). These and similar suitable methods are known in the art.

In a subject having a disease or disorder involving an epithelial surface, a localized immune response may be induced by applying one or more immune response modifiers, or composition comprising same to the affected areas of the epithelial surface. Without wishing to be bound by theory, compositions according to some embodiments of the invention may induce maturation of some immune cells or may induce clonal expansion of some immune cells, or may induce cytokine production in some immune cells at or near the epithelial surface to which they are applied. The cytokines trigger the subject's immune system to recognize the presence of a viral infection or neoplasm, and the associated lsion may be eradicated in an immunogen-independent manner. Alternately, the composition may comprise one or more specific immunogens in combination with an immune response modifier, and the immune response may thus further comprise immunogen-specific responses (e.g. specific T cell or antibody responses). As exemplified in Table 1, stimulation of selected TLRs, such as TLR3 or TLR8 induce localized production of interferons (for example interferon alpha, beta or gamma) and interleukins (for example IL-5, or IL-4) and maturation of dendritic cells, which in turn may assist in localized activation of other immune cells such as T cells, natural killer cells and the like.

The invention, therefore, provides for methods of inducing cytokine production at or near an epithelial surface in the absence of an immunogen. The invention further provides for methods of stimulating immune cells, including dendritic and CD8+ cells, at or near an epithelial surface.

For example a method of inducing a localized immune response at or near an epithelial surface, according to some embodiments of the invention, may comprise applying a topical preparation or composition comprising one, or more than one of the following: polyIC and a cationic polymer, and a topical base; the cationic polymer comprising from about 100 to about 700 amino acid residues, wherein the mass:mass ratio of the polyIC:cationic polymer is from about 4:1.4 to about 4:3; and wherein the topical base may be one or more than one of a cream, ointment, oil, gel, paste, powder, lotion, liniment, emulsion, or fatty acid, emollient, emulsifier, thickener, preservative.

In some embodiments of the invention, an immunogen may be an allergen. An allergen is an agent that induces an allergic response in a subject, upon exposure to the allergen. Chronic inflammation observed in allergic and asthmatic disorders resulting from inhaled allergens is largely dominated by localized tissue infiltration of eosinophils, and hyperreactivity of the tissues to the allergen. Inflammation may be reduced through use of corticosteroids and/or bronchodilators, however these do not treat the root cause. As discussed in WO 99/07860, allergen-specific T-lymphocytes are selectively enriched in such hyperreactive tissue, and this sensitivity may be dependent on early antigen exposure in childhood or infancy.

Selection for specific Th1-versus Th2-like memory cells in an individual immune response to inhaled antigens occurs in the regional lymph nodes draining the conducting airways. Such a selection may be regulated by a variety of cytokines produced by antigen specific CD4+ and CD8+ T-cells. The T-cell selection process may be influenced by infectious agents: infections in the airway mucosa may mobilize and activate local tissue (alveolar) macrophages which migrate to the regional lymph nodes and secrete Th2 inhibitory cytokines such as IL-12 and alpha-interferon. In addition, they may add to the gamma-interferon levels in the milieu through activation of natural killer cells. The net result is the production of CTLs (which are predominantly CD8+ cells). Gamma-interferon inhibits the generation of Th2 cells and therefore production of IL-4 and IL-5, cytokines crucial for the generation of humoral (IgE) and cellular (eosinophils, basophils and mast cells) allergic responses (Anderson, G. P. and Coyle, A. J., Trends Pharmacol. Sci., 15:324-332 (1995); Stam, W. B., van Oosterhout, A. J. and Nijkamp, F. P., Life Sci., 53:1921-1934 (19939)).

In mammals, stress proteins have been shown to induce humoral as well as cellular immune responses. When a soluble antigen mixed with, chemically conjugated to, or fused to a stress protein is administered to a mammal, cell-mediated cytolytic immune responses are substantially enhanced. These responses are largely due to CD8+ T cells. Therefore, a comparison of the CD4+ response to antigens by themselves with the CD4+ response to antigens mixed with or coupled to a stress protein will give the predicted profile: soluble antigens mixed with or linked to stress proteins yield a high proportion of CTLs (mainly CD8+ T cells) which are a measure of stimulation of the Th1 pathway described before because these CTLs arose as a result of the induction of antigen specific T cells of the Th1 type. These Th1 cells produce gamma-interferon, which inhibits Th2 cells. Therefore, the Th2 cytokines IL-4 and IL-5 are no longer available to support the production of IgE and eosinophils. With decreasing titer of IgE, direct antigenic stimulation of mast and basophil cells will decline. In addition, decreased IL-5 production will lead to decreased production, differentiation and activation of eosinophils. This pattern will cause decreased inflammation of the involved tissue and result in less hyperreactive (asthmatic) events.

Therefore, administration of mixtures of known allergenic antigens (allergens), or stress proteins or compositions comprising allergens chemically linked to or fused to stress proteins in combination with agents according to Formula II, IIa-d, Formula III, IIIa-d, Formula IVa-j, or combinations of at least two of Formula IVa-j in various molar ratios may influence the Th1 to Th2 ratio in atopic patients, restoring a more normal balance and leading to decreased allergic or asthmatic response.

In some embodiments of the invention, the adjuvant composition may be a selective agonist for TLR8 or TLR3. In some embodiments of the invention the adjuvant composition may be an agonist for both TLR8 and TLR3.

Therefore, the invention provides for a TLR8 agonist or a TLR3 agonist comprising polyIC and a cationic polymer, the cationic polymer comprising from about 100 to about 700 amino acid residues and the mass: mass ration of polyIC:cationic polyumer is from about 4:14 to about 4:3 mg/ml, or from about 4.15 to about 4:2 mg/ml. The polycationic polymer may be polyarginine.

Adjuvant compositions, or some components of adjuvant compositions according to some embodiments of the invention may be made, in part, using enzymatic methodologies. For example, polymers of inosine (polyI) or cytosine (polyC may be synthesized using chemical or enzymatic methods, such as are known in the art. An example of enzymatic synthesis is described. Inosine diphosphate (IDP, mw 494.3) or cytosine diphosphate (CDP, mw 505.15) as a 400 mg/ml solution in suitable buffer is combined with polynucleotide polymerase (PNPase) (˜10 units per mg IDP or CDP) and incubated at 37° C. for 20-24 hours. Proteinase K (1 mg/2000 units PNPase) was subsequently added and incubated for a further ˜2 hours at 37° C. with gentle agitation. The solution was brought to a final concentration of ˜0.35 M NaCl (e.g. using a 5 M NaCl stock), and 1-5 volumes of absolute ethanol added and mixed gently. The resulting mixture was held at 0° C. for 8-24 hours. The precipitants were separated by filtration, dissolved in water and re-precipitated in 1-5 volumes absolute ethanol, as previously. Following filtration, the solids were dissolved in water, diafiltered with water to remove ions, and lyophilized until needed. MW of the resulting polymer may be determined, for example, by SEC-MALLS.

Once the polyI and polyC polymers are obtained, polyIC complexes may be obtained by hybridization. An example of such a method is described. Solutions of 6 mg/ml of each polyI and polyC are combined with gentle mixing and heated to 70-75° C. for ˜1 hr, and allowed to cool slowly to room temperature with continued mixing for 8-24 hours. The resulting mixture is filtered to remove insoluble components and diafiltered with water to remove ions, and lyophilized until needed. MW of the resulting polymer may be determined, for example, by SEC-MALLS.

To prepare a composition of polyIC/R, a solution of polyarginine (polyR) in suitable buffer is prepared and filtered, and a solution of polyIC is combined with the polyR solution slowly with agitation (e.g. dropwise, with mixing). The polyIC and polyR may be present in a mass:mass ratio from about 4:1 to about 4:3.

If smaller MW polyIC is desired (e.g. ˜330 kDa), an additional incubation step at 60-70° C. with agitation for 2-6 hours followed by cooling in an ice bath may be included in the above methods, before addition of NaCl and precipitation. Time of incubation is monitored by periodically sampling the solution and determining average MW of the polymer.

Examples of suitable buffers include phosphate buffer, HEPES, PBS, glycine or other enzymatically-compatible buffer that maintains a pH in the range of 6.5 to 9.0. Buffers and/or water used in these methods may be RNAse-free water.

Therefore, the invention provides for methods of making adjuvant compositions comprising polyIC and a cationic polymer.

Methods and Materials

Sources of materials—polyR, polyIC, polyICLC

Polyarginine (15-70 kDa MW) was obtained from Sigma. The procedure comprised guanylation of poly-L-ornithine(HBr) using 3,5-dimethylpyrazole-1-carboxamidine nitrate. The reaction was cooled to room temperature and treated with 2N HCl; and the solids filtered off. The resulting solution was filtered using a Pellicon 2 TFF System equipped with 2×0.5 m²1K regenerated cellulose membrane. The purified poly-L-arginine was subsequently filtered sterilized and lyophilized. Poly IC was obtained from Sigma.

Manufacture of Poly IC/R

Large MW PolyIC (−737 kDa)

Preparation of Large Molecular Weight Polyinosinic Acid (Poly I, WO893, Lot # 18K4214)

Prepare a solution of inosine diphosphate (IDP. Sigma, 57490, mw 494.3) (˜40 gm) in 400 ml of 0.1M HEPES, pH 7.0 buffer. Add 60 ml of polynucleotide polymerase (PNPase, Sigma) (69.5 units/ml) to IDP solution, incubate reaction mixture at 37° C. for 20-24 hours. Add 2 ml of Proteinase-K (@ 1 mg/ml soln) to reaction mixture and incubate for 2 hours at 37° C. with gentle agitation, cool in ice-bath. Add 15-16 ml of 5M NaCl, mix; add 1-5 volume absolute ethanol, and hold in ice-bath 8-24 hours. Filter solids and dissolve solids in water; repeat alcohol precipitation step. Dissolve solids in water and diafilter against water (Millipore, 30,000 MWCO, 0.1 m², Regen Cellulose) membrane; lyophilize (yield: ˜9 grams, average MW 1,033 kDa).

Preparation of Large Molecular Weight Polycytidinic Acid (Poly C, WO768, Lot # 18K4213)

Prepare a solution of cytidine diphosphate (CDP; Sigma, 30280, mw 505.15) (—P40 gm) in 400 ml of 0.1M HEPES, pH 7.0 buffer. Add 60 ml of polynucleotide polymerase (PNPase, Sigma) (69.5 units/ml) to CDP solution, incubate reaction mixture at 37° C. for 20-24 hours. Add 2 ml of Proteinase-K (@ 1 mg/ml soln) to reaction mixture and incubate for 2 hours at 37° C. with gentle agitation, cool in ice-bath. Add 15-16 ml of 5M NaCl, mix; add 1-5 volume absolute ethanol, and hold in ice-bath 8-24 hours. Filter solids and dissolve solids in water; repeat alcohol precipitation step. Dissolve solids in water and diafilter against water (Millipore, 30,000 MWCO, 0.1 m², Regen Cellulose) membrane; lyophilize (yield: ˜12 grams, average MW 745 kDa).

Preparation of Large Molecular Weight Poly I: Poly C Complex (WO643, Lot # 18K4228)

Dissolve 5.02 gm of Poly I (Sigma WO893, Lot 18K4214, or prepared as above) in 785 ml of 1×PBS buffer at 60-65° C.; dissolve 4.66 gm of Poly C (WO768, Lot 18K4213, or prepared as above) in 767 ml of 1×PBS buffer at 60-65° C. Combine poly I and polyC solution with gentle mixing, heat to 70-75° C. with agitation for 1 hr; cool to room temperature with continued agitation for 8-24 hours. Filter the solution through Whatman 54 and glass filter paper; followed by diafiltration against water (Millipore, 30,000 MWCO, 0.1 m², Regen Cellulose) membrane, lyophilize (yield ˜9 gm; 737 kDa).

Preparation of Poly IC: Poly Arginine Complex (polyIC/R)(Lot # 18K4229)

Prepare 8 ml of a 1 mg/ml solution of Poly Arginine (Sigma, W1018, Lot 107K4220)) in 1×PBS, hold at 2-8° C. overnight, filter (0.22 um filter paper). Prepare 4 ml of a 4 mg/ml solution of Poly IC (WO643, Lot 18K4228) in RNAase free water. Gradually (over 1-2 hours) combine the poly arginine with the polyIC solution with agitation (yield 12 ml of polyIC/R)

Smaller MW polyIC (−330 kDa)

Preparation of Smaller Molecular Weight Polyinosinic Acid (Poly I, WO893, Lot # 28K4208)

Prepare a solution of inosine diphosphate (IDP. Sigma, 57490, mw 494.3) (−40 gm) in 400 ml of 0.1M HEPES, pH 7.0 buffer. Add 60 ml of polynucleotide polymerase (PNPase, Sigma) (69.5 units/ml) to IDP solution, incubate reaction mixture at 37° C. for 20-24 hours. Add 2 ml of Proteinase-K (@ 1 mg/ml soln) to reaction mixture and incubate for 2 hours at 37° C. with gentle agitation, transfer to 60° C. water bath and incubate until target MW of polymer is achieved (−2-6 hours); cool in ice-bath. Add 15-16 ml of 5M NaCl, mix; add 1-5 volume absolute ethanol, and hold in ice-bath 8-24 hours. Filter solids and dissolve solids in water; repeat alcohol precipitation step. Dissolve solids in water and diafilter against water (Millipore, 30,000 MWCO, 0.1 m², Regen Cellulose) membrane; lyophilize (yield: ˜15 grams, average MW 291.5 kDa).

Preparation of Smaller Molecular Weight Polycytidinic Acid (Poly C, WO768, Lot # 28K4209)

Prepare a solution of inosine diphosphate (CDP Sigma, 30280) (−40 gm) in 400 ml of 0.1M Glycine pH 9.0. Add 60 ml of polynucleotide polymerase (PNPase, Sigma) (69.5 units/ml) to IDP solution, incubate reaction mixture at 37° C. for 20-24 hours. Add 2 ml of Proteinase-K (@ 1 mg/ml soln) to reaction mixture and incubate for 2 hours at 37° C. with gentle agitation, transfer to 70° C. water bath and incubate until target MW of polymer is achieved (−2-6 hours); cool in ice-bath. Add 15-16 ml of 5M NaCl, mix; add 1-5 volume absolute ethanol, and hold in ice-bath 8-24 hours. Filter solids and dissolve solids in water; repeat alcohol precipitation step. Dissolve solids in water and diafilter against water (Millipore, 30,000 MWCO, 0.1 m², Regen Cellulose) membrane; lyophilize (yield: ˜15 grams, average MW 217 kDa).

Preparation of Smaller Molecular Weight Poly I: Poly C Complex (WO643, Lot # 28K4215)

Dissolve 5.33 gm of Poly I (Sigma WO893, Lot 28K4208, or prepared as above) in 840 ml of 1×PBS buffer at 60-65° C.; dissolve 5.00 gm of Poly C (WO768, Lot 28K4209, or prepared as above) in 823 ml of 1×PBS buffer at 60-65° C. Combine poly I and polyC solution with gentle mixing, heat to 70-75° C. with agitation for 1 hr; cool to room temperature with continued agitation for 8-24 hours. Filter the solution through Whatman 54 and glass filter paper; followed by diafiltration against water (Millipore, 30,000 MWCO, 0.1 m², Regen Cellulose) membrane, lyophilize (yield ˜10 gm; MW 330.4 kDa).

### Preparation of PolyIC:Poly Arginine Complex (small MW poly IC/R) (Lot # 28K4222)

Prepare ˜25 ml of a 1 mg/ml solution of Poly Arginine (Sigma, W1018, Lot 107K4220)) in 1×PBS, hold at 2-8° C. overnight, filter (0.22 um filter paper). Prepare ˜12 ml of a 4 mg/ml solution of Poly IC (WO643, Lot 28K4215) in RNAase free water. Combine the poly arginine with the polyIC solution, with agitation for 45-60 minutes (yield 12 ml of polyIC/R).

HT-29 IP-10 Assay

HT-29 cells (ATCC HTB-38) were used for quantitative measurement of human IP-10 using a sandwich ELISA assay (Peprotech Cat # 900-K39). HT29 cells were seeded into 96-well plate with cell density of 100,000 to 200,000 cells per well. Plates were incubated overnight into a 37° C. incubator at 5% CO₂ Agonist mixtures having final concentrations from 0.4 to 50 ug/ml were prepared, and combined with 20% of Human AB sera (SIGMA Cat # H4522) in DMEM or RMPI 1640 (Mediatech 10-040CM), and incubated for 24 or 48 hours at room temperature before being added to the plated HT29 cells.

At Day 2, old media from the 96-well plate of HT29 cells were aspirated off and 200 ul of each serial dilutions were added per well. Cells with different concentrations of adjuvants were incubated overnight into a 37° C. incubator at 5% CO₂.

Capture antibody (100 ug of antigen-affinity purified rabbit anti-human IP-10) were reconstituted in 1 ml sterile water for a concentration of 100 ug/ml and subsequently diluted with PBS to a working concentration of 1 ug/ml. Maxisorp ELISA plates were coated with 100 ul of the diluted capture antibody. The plates were sealed and incubated at room temperature for at least 2 hours or at 4° C. overnight. The liquid were then removed from the wells and the plates were washed 4 times using 300 ul of wash buffer (0.05% Tween-20 in PBS) per well. After each wash, the plates were blotted on absorbent paper to remove residual buffer. About 300 ul of block buffer (1% BSA in PBS) were added to each well. Plates were sealed and incubated for at least 1 hour at room temperature or at 4° C. overnight. After the blocking time was achieved, the plates were washed with wash buffer for four (4) times.

Human IP-10 standard were reconstituted in 1 ml sterile water for a concentration of 1 ug/ml. Standard curves were prepared in diluent buffer (0.05% Tween-20, 0.1% BSA in PBS) from 2000 pg/ml to 3.9 pg/ml and 100 ul were added per well in duplicate wells. Supernatants from HT29 cells were harvested, diluted at 1:4 and were added in triplicate to the ELISA plates. The plates were sealed and incubated at room temperature for 1 to 2 hours. The liquid were then aspirated off and plates were washed with wash buffer for four (4) times.

Detection antibody (biotinylated antigen-affinity purified rabbit anti-human IP-10) was reconstituted in 0.25 ml sterile water for a concentration of 100 ug/ml. It was then diluted in diluent to a concentration of 0.25 ug/ml, and 100 ul were added per well. Plates were sealed and incubated at room temperature for 1 to 2 hours. The liquid were then aspirated off and plates were washed with wash buffer for four (4) times.

Avidin-HRP conjugate were diluted to 1:2000 in diluent buffer or as specified on the lot-specific kit instructions, and 100 ul were added per well. Plates were sealed and incubated at room temperature for 20 to 30 minutes. The liquid were then aspirated off and plates were washed with wash buffer for four (4) times.

ABTS liquid substrate (2,2′-Azino-Bis(3-ethylbenzthiazoline-6-sulfonic acid) was then added at 100 ul per well. Plates were incubated at room temperature for color development for about 5 to 10 minutes. Stop solution (1% SDS) were then added at 100 ul per well. Plates were read using an ELISA plate reader at 405 nm.

Human PBMC Cytokine Assay

DMEM containing 10% FBS was warmed up. About 20 units/ml of DNaseI was added to the media. Cryopreserved PBMC (about 50 million cells per vial) was thawed at 37° C. water bath, and aseptically transferred into a 50-ml conical tube. The vial was rinsed with 1 ml of warm culture media and then was added dropwise to the cells while gently swirling the tube. Medium was slowly added to the cells until the total volume is 50 ml. The cell suspension was centrifuged at 200×g for 15 minutes at room temperature. All but 2 ml of the wash was carefully removed by pipet. The cell pellet was carefully resuspended in the remaining 2 ml of the media. Another 8 ml of the media was added for a total of 10 ml of cell suspension.

Serial dilutions of poly-IC-polyR, poly-ICLC and poly-IC were prepared, to provide a final concentration of 50, 25, 12.5, 6.25, 3.1, and 1.6 ug/ml per well. 500 ul of each dilution was added to each well on a 24-well plate, and 500 ul of the PBMC cell suspension added to each well. Plates were incugated in a 37° C. incubator at 5% CO₂ for 8 and 24 hours.

Cytokines were detected and quantified using kits (ELISA or bead multiplexing) from Millipore or R&D systems, following manufacturer's instructions.

Capture antibodies (IFNalpha, IFNbeta, IFNgamma) were diluted with PBS to each assigned concentration. Maxisorp ELISA plates were coated with 100 ul of specific anti-cytokine antibodies, sealed with plate sealing tape and incubated at room temperature for at least 2 hours or at 4° C. overnight. The liquid were then removed from the wells and the plates were washed 4 times using 300 ul of wash buffer (0.05% Tween-20 in PBS) per well. After each wash, the plates were blotted on absorbent paper to remove residual buffer. About 300 ul of block buffer (1% BSA in PBS) were added to each well. Plates were sealed and incubated for at least 1 hour at room temperature or at 4° C. overnight. After the blocking time was achieved, the plates were washed with wash buffer for four (4) times.

Standard curves for each of the human cytokines (IFNgamma, TNF alpha, IL-6, IL-8, IL-10, IL-12 p70) were prepared as specified on the lot-specific kit instructions and 100 ul were added per well in duplicate wells. The PBMC cells plus supernatant were collected and spun down to pellet the cells. Supernatants were harvested and 100 ul were added in triplicate to the ELISA plates coated with specific anti-cytokine capture antibodies. The plates were sealed and incubated at room temperature for 1 to 2 hours. The liquid were then aspirated off and plates were washed with wash buffer for four (4) times.

Detection antibody for each specific cytokine was diluted in diluent buffer (0.05% Tween-20, 0.1% BSA in PBS) or as specified on the lot-specific kit instructions, and 100 ul were added per well. Plates were sealed and incubated at room temperature for 1 to 2 hours. The liquid were then aspirated off and plates were washed with wash buffer for four (4) times.

Avidin-HRP conjugate were diluted to 1:2000 in diluent buffer (0.05% Tween-20, 0.1% BSA in PBS) or as specified on the lot-specific kit instructions, and 100 ul were added per well. Plates were sealed and incubated at room temperature for 20 to 30 minutes. The liquid were then aspirated off and plates were washed with wash buffer for four (4) times. Substrate solution (dependent on the ELISA kit used) was then added at 100 ul per well. Plates were incubated at room temperature for color development for about 10 to 20 minutes. Stop solution were then added at 50 to 100 ul per well. Plates were read using an ELISA plate reader at 450 nm.

Dendritic Cell Maturation

### Preparation of Immature Dendritic Cells

Cells were grown in RPMI supplemented with 10% Fetal Bovine Serum and 20 U/ml Dnasel (Sigma). Peripheral Blood CD14+Monocytes Cells (Lonza) were treated with 50 ng/ml of Human GM-CSF and Human IL-4 (PeproTech) and were plated in four 8 wells of 12 well plates at 1 million cells/mL. On day 3 non adherent cells were removed and 2 mL of fresh culture medium treated with 50 ng/ml of Human GM-CSF and Human IL-4 were added to the culture and incubate at 37° C. CD14+ cells begin to differentiate into immature dendritic cells on day 7 and were detected by various dendritic cell markers by flow cytometry.

Preparation of Mature Dendritic Cells

Immature Dendritic cells were cultured for 48 hours with 3 different adjuvants to differentiate into mature dendritic cells. 5 ug/ml of each adjuvants were added to each plate and incubate at 37° C. After 48 hours, cells were stained with different dendritic cells markers and detected by flow cytometry.

### Immunofluorescent Staining of Cell Surface Antigens for Flow Cytometric Analysis

Cell preparation: Cells were removed from each plate and trypsanized. Each well were pooled and wash once with PBS. Cells were centrifuge for 5 minutes at 2000 g and resuspended in 500 ul of Flow Stain Buffer.

Cell Surface Expression

Each set of cells were stained with dendritic cell markers, anti-human CD11c FITC, anti-human HLA-ABC PE, anti-human HLA-DR PE, and anti-human CD86 PE (eBioscience) and analyzed by FACScan and Cell Quest Software (Becton Dickinson).

Serum Stability/Serum Pretreatment

Non-heat inactivated human AB serum was used in pretreatment of adjuvant examples polyIC, polyIC/R and/or polyICLC. The prepared adjuvants were added to 20% non-heat inactivated serum and incubated overnight (18-24 hours) (at what temperature or other conditions?) Following serum pretreatment, the adjuvant preparation was used in the assays as described.

Rnase A Hydrolysis Assay

Poly IC at 4 mg/ml was mixed with 1.6 or 2 mg/ml polylysine, or 1.5 or 2 mg/ml polyarginine and allowed to incubate for 30 minutes with constant stirring, allowing a complex of polyIC and polycationic polymer (polyarginine or polylysine) to form. Aliquots of the complexes were exposed to 2.5 ug RnaseA and samples taken at various time points. Each sample was assayed for absorbance at 260 nm, and the absorbance plotted against time.

TLR-3 and TLR-8 Agonist Assay

The HEK-293 cell line (ATCC, CRL-1573) is a permanent line of primary human kidney transformed by sheared human adenovirus type 5 DNA. 293XL-hTLR8 cells (InvivoGen, San Diego, Calif.) were obtained by transfection of the human TLR8 gene into HEK293 cells expressing the human Bcl-XL (anti-apoptosis) gene. 293-hTLR3 cells (InvivoGen, San Diego, Calif.) were obtained by transfection with the plasmid pUNO-hTLR3, and then isolated clones were picked and tested for expression of human TLR3.

HEK 293 cells and 293XL-hTLR8 or 293-hTLR3 cells were seeded into 96-well plate with cell density of 100,000 cells per well. Plates were incubated overnight into a 37° C. incubator at 5% CO₂. At Day 2, tubes with 10% of FBS in DMEM were prepared. The amount of Polycytidylic acid (Poly C—Sigma Cat #P4903), Polyinosinic acid (Poly I—Sigma Cat #P4154), Poly-L-arginine (Poly R), Poly IC/Poly R, PolyICLC and Poly IC were calculated and added to each tube to have final concentrations of 25 ug/ml. Old media were aspirated off and 200 ul of each concentration of different PolyICs and its analogues were added in triplicate wells for both HEK 293 and 293XL-hTLR8 cells or 293-hTLR3. Cells with different concentrations of adjuvants were incubated overnight into a 37° C. incubator at 5% CO₂.

After an overnight incubation, immunoassay was performed on cells' supernatants using Biosource's Multiplex bead assay on Human IP-10 (Cat # LHC1081). The assay was performed in a 96-well plate format and analyzed with the Luminex instrument. The ability of the agonist (polyIC/R, poly ICLC, polyIC or controls) to stimulate NF kappa B as a direct result of TLR receptor engagement induce the release of SEAP or secreted alkaline phosphatase (y axis).

ELISpot Assay for Murine IFN-Gamma Production in Splenocytes

Mice were immunized with PBS, with HspE7 alone, or 500 ug of HspE7 plus 100 ug of either PolyICR or PolyIC. After 7 days post immunization, mice were sacrificed and spleens were removed and processed to a single cell suspension. Detection of IFN-gamma secreting cells was performed using an ELISPOT assay (R&D Systems Cat # EL485), following manufacturer's instructions.

Briefly, a monoclonal antibody specific for mouse IFN-γ was pre-coated onto a PVDF (polyvinylidene diflouride)-backed microplate. A single cell suspension of splenocytes was loaded into the microplate at cell density of 2×10⁵ cells per well. Splenocytes were restimulated for 24 hours with media only, Flu NP (Negative control MHC I peptide) or HPV 16 E7₄₉₋₅₇ peptide and the microplate was placed into a humidified 37° C. CO₂ incubator overnight. Following incubation, wells were washed, and a biotinylated polyclonal antibody specific for mouse IFN-γ was added to the wells. Following a wash to remove any unbound biotinylated antibody, alkaline-phosphatase conjugated streptavidin was added. Unbound enzyme was subsequently removed by washing and a substrate solution (BCIP/NBT) was added. A blue-black colored precipitate formed at the sites of the cytokine localization and appeared as spots, with each individual spot representing an individual IFN-γ secreting cell. The spots were counted with an automated ELISPOT reader system using a stereomicroscope.

Tumor Regression/Prophylaxis Assays

TC-1 cells (ATCC CRL-2785) were derived from primary lung epithelial cells of C57BL/6 mice and are positive for the expression of HPV-16 E6 and E7. TC-1 Kast (TC-1K) cells were selected by their demonstration of a more aggressive tumor growth patter in C57B1/6 mice than TC-1 cells.

In preparation for implantation into mice, TC-1Kast and E.G7-OVA cells were cultured until approximately 90% confluent and harvested by washing with DPBS (Mediatech MT21-030-CM) and trypsinization with 025% trypsin (BD 90000-902). Cells were harvested and diluted with RPMI 1640 (MediatechMT10-040-CV). The tumor cells were pelleted at 1000 g, washed twice in RPMI 1640 and adjusted to 5×10⁵ viable cells per ml (or 1×10⁵ per 200 ul) as determined by trypan blue dye exclusion. The viability of tumor cells implanted into mice should always be >90%. For tumor establishment, 200 ul of TC-1Kast or E.G7-OVA cells were implanted subcutaneously on the hind flank of female C57BL/6 mice (7 to 8 weeks old) using a 25 gauge needle. After 7 days, the area was observed for the presence of a tumor nodule.

For tumor regression assays, tumor cells were implanted at day zero. Mice were immunized once at day 7 when all mice had palpable tumors. Single treatment of HspE7 (400 ug) alone, or OVA (400 ug) alone, or PolyIC/R (100 ug) alone, or HspE7 or OVA plus various dosage of PolyIC/R (100, 50 or 25 ug) were given to each group of mice. All immunizations were administered subcutaneously in the scruff of the neck in total volume of 0.2 ml. Naïve mice were injected with 200 ul of 1×PBS.

Mice were monitored for the tumor incidence every 2 days post immunization.

For the tumor development inhibition (“prophylaxis”) assays, mice were immunized once at day 0. A single treatment of HspE7 (400 ug) alone, or PolyIC/R (100 ug) alone, or HspE7 plus various dosage of PolyIC/R (100, 50 or 25 ug) were given to each group of mice. All immunizations were administered subcutaneously in the scruff of the neck in total volume of 0.2 ml. Naïve mice were injected with 200 ul of 1×PBS. Tumor cells were implanted as described on day 7.

Antigen-Specific Antibody Response

Mice were administered on day 1 and day 21 a dose of 100 ug poly IC/R (100 ug), HspE7 (400 ug), a combination of HspE7 (400 ug)+poly IC/R (100 ug) in the presence or absence of alum (how much?). Naïve mice were injected with 200 ul of 1×PBS. Serum samples were obtained by tail bleed on day 42. Following coagulation and removal of cells, serum samples were diluted 1:100 for assaying. HspE7-coated 96-well ELISA plates were blocked with 1% BSA in PBS, and dilutions of the murine serum in 0.05% Tween-20, 0.1% BSA in PBS were added to the wells, plates were sealed and incubated for at least 1 hour at room temperature or at 4° C. overnight. Plates were washed 4× with wash buffer (0.05% Tween-20 in PBS). After each wash, the plates were blotted on absorbent paper to remove residual buffer.

Following washing, Avidin-HRP conjugate were diluted to 1:2000 in diluent buffer (0.05% Tween-20, 0.1% BSA in PBS) or as specified on the lot-specific kit instructions, and 100 ul were added per well. Plates were sealed and incubated at room temperature for 20 to 30 minutes. The liquid was aspirated off and plates washed 4× with wash buffer. Substrate solution (dependent on the ELISA kit used) was then added at 100 ul per well. Plates were incubated at room temperature for color development for about 10 to 20 minutes. Stop solution were then added at 50 to 100 ul per well. Plates were read using an ELISA plate reader at 450 nm.

### Example 1

### Preparation of Double-Stranded Oligomers: GCLNA-PolyIC-GCLNA:

Oligomers according to SEQ ID NO: 1 and SEQ ID NO: 2 were synthesized using 2′-OMe-1-CE Phosphoramidites, 2′-OMe-C-CE Phosphoramidites, 5-Me-Bz-C-LNA-CE phosphoramidites and dmf-G-LNA-CE phosphoramidites according to standard techniques, as per manufacturer's protocols (Glen Research, Sterling Va.).

| | |
|---|---|
| G_{LNA}-G_{LNA}-(I₂₂)-G_{LNA}-G_{LNA} | (SEQ ID NO: 1) |

and

| | |
|---|---|
| C_{LNA}-C_{LNA}-(C₂₂)-C_{LNA}-C_{LNA} | (SEQ ID NO: 2) |

Equimolar amounts of each of the first and second oligomers were combined and permitted to anneal to produce the dsRNA compound GCLNA-polyIC-GCLNA, shown in Formula IIIa:

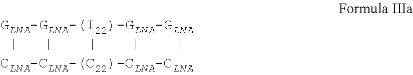

### Example 2

### Preparation of Double-Stranded Oligomers with 3′ Unpaired Ends

Oligomers according to SEQ ID NO: 3, SEQ ID NO: 4, SEQ ID NO:5, SEQ ID NO: 6, SEQ ID NO: 7, and SEQ ID NO: 8 may be synthesized using 5-Me-Bz-C-LNA-CE Phosphoramidites, Bz-A-LNA-CE Phosphoramidites, dmf-G-LNA-CE Phosphoramidites, T-LNA-CE Phosphoramidites, 2′-OMe-1-CE Phosphoramidites, 2′-OMe-C-CE Phosphoramidites, 2′-OMe-A-CE Phosphoramidites, 2′-OMe-G-CE Phosphoramidites and 2′-OMe-U-CE Phosphoramidites according to standard techniques, as per manufacturer's protocols (Glen Research, Sterling Va.).

(SEQ ID NO: 3)

(I₁₅)-G-T_{LNA}-G_{LNA}-A-T_{LNA}-A-T_{LNA}-G_{LNA}

(SEQ ID NO: 4)

(C₁₅)-C_{LNA}-A-T_{LNA}-A-T_{LNA}-C-A_{LNA}-C_{LNA}

(SEQ ID NO: 5)

G_{LNA}-(I₁₅)-G-T_{LNA}-G_{LNA}-A-T_{LNA}-A-T_{LNA}

(SEQ ID NO: 6)

C_{LNA}-(C₁₅)-C_{LNA}-A-T_{LNA}-A-U-C_{LNA}-A_{LNA}

(SEQ ID NO: 7)

T_{LNA}-G_{LNA}-(I₁₅)-T_{LNA}-T_{LNA}-A-T_{LNA}-A_{LNA}

(SEQ ID NO: 8)

A_{LNA}-C_{LNA}-(C₁₅)-C_{LNA}-A-T_{LNA}-A-T_{LNA}-C_{LNA}

Equimolar amounts of each of SEQ ID NO: 3 and SEQ ID NO: 4, or SEQ ID NO:5 and SEQ ID NO: 6 or SEQ ID NO: 7 and SEQ ID NO: 8 may be combined and permitted to anneal to produce the double-stranded nucleic acid compounds shown in Formula Va, Vb and Vc, respectively.

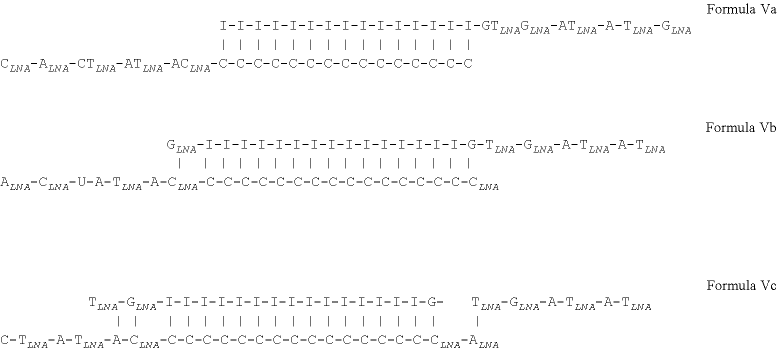

### Example 3

### Preparation of Double-Stranded Oligomers with 3′ Unpaired Ends

Oligomers according to SEQ ID NO: 9, SEQ ID NO: 10, SEQ ID NO:11 and SEQ ID NO: 12 may be synthesized using 5-Me-Bz-C-LNA-CE Phosphoramidites, Bz-A-LNA-CE Phosphoramidites, dmf-G-LNA-CE Phosphoramidites, T-LNA-CE Phosphoramidites, 2′-0Me-I-CE Phosphoramidites, 2′-OMe-C-CE Phosphoramidites, 2′-OMe-A-CE Phosphoramidites, T-OMe-G-CE Phosphoramidites and 2′-OMe-U-CE Phosphoramidites according to standard techniques, as per manufacturer's protocols (Glen Research, Sterling Va.).

| | |
|---|---|
| G_{LNA}-G_{LNA}-(I)₁₅-(A)₁₅ | (SEQ ID NO: 9) |
| (C)₁₅-C_{LNA}-C_{LNA}-(U)₁₅ | (SEQ ID NO: 10) |
| G_{LNA}-G_{LNA}-(I)₁₀-(A)₁₀ | (SEQ ID NO: 11) |
| (U)₁₀-C_{LNA}-C_{LNA}-(C)₁₀ | (SEQ ID NO: 12) |

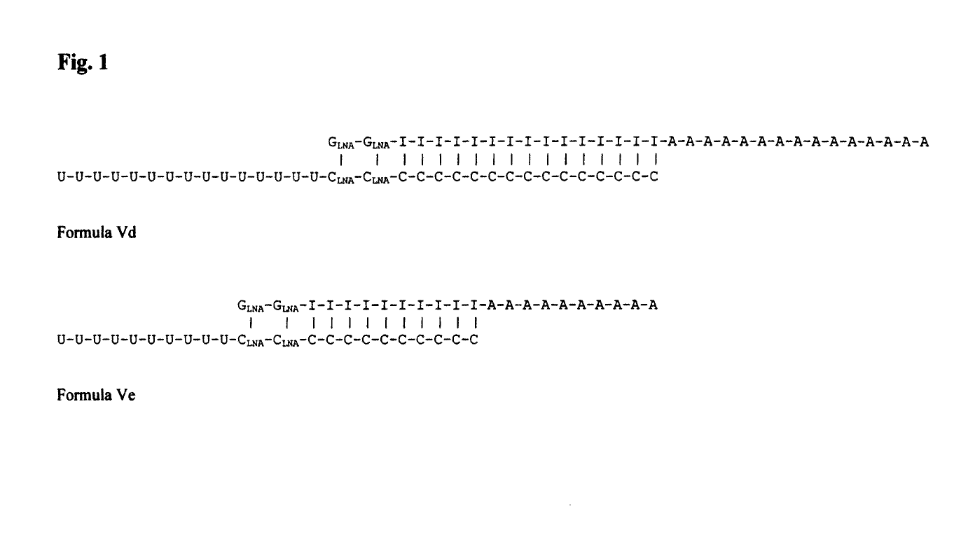

Equimolar amounts of each of SEQ ID NO: 9 and SEQ ID NO: 10, or SEQ ID NO:11 and SEQ ID NO: 12 may be combined and permitted to anneal to produce the double-stranded nucleic acid compounds shown in Formula Vd and Ve, respectively (FIG. 1).

### Example 4

### In Vitro Biological Activity of dsRNA in Combination with an Immunogen

A composition comprising HspE7, produced according to the method of U.S. 60/803,606 (which is incorporated herein by reference) and GCLNA-polyIC-GCLNA produced according to Example 1 above, may be tested for biological activity in vitro.

Augmentation of the ability of HspE7 to induce E7-specific CD8-positive T-lymphocytes (as an exemplary antiviral therapeutic approach) may be determined in the presence of GCLNA-polyIC-GCLNA. Naïve C57B1/6 mice may be injected subcutaneously, with either HspE7 alone, or HspE7 plus GCLNA-polyIC-GCLNA. After a time interval, for example 5 days, spleens may be removed from the mice and the number of E7-specific splenocytes measured by ELISPOT, for example, by using E7 specific class I MHC binding peptide E749-57 (RAHYNIVTF; Dalton Chemical Laboratories), or a control peptide HBCAg93-100 (MGLKFRQL; Dalton Chemical Laboratories) as recall antigens.

### Example 5

### In Vivo Biological Activity of dsRNA in Combination with an Immunogen

A composition comprising HspE7, produced according to the method of U.S. 60/803,606 (which is incorporated herein by reference) and GCLNA-polyIC-GCLNA produced according to Example 1 above, may be tested for biological activity in vivo.

In an exemplary method of a cancer therapeutic method, TC-1 tumors are first established in naive C57B1/6 mice. Mice were injected in the flank with 6×10⁴ TC-1 tumor cells. On day 7, mice bearing established TC-1 tumors may be injected subcutaneously in the scruff of the neck with either diluent, purified HspE7 alone, or graded doses of purified HspE7 mixed with different doses of GCLNA-polyIC-GCLNA. Mice are followed for tumor growth for an additional time interval, for example, 42 days—in this example, mice free of tumor 49 days post tumor implantation may be considered to be tumor free.

### Example 6

### Preparation of Double-Stranded Oligomers Comprising CpG Motifs

Oligomers according to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21 and SEQ ID NO: 22 were synthesized using 2′-OMe-1-CE Phosphoramidites, 2′-OMe-C-CE Phosphoramidites, 5-Me-Bz-C-LNA-CE phosphoramidites and dmf-G-LNA-CE phosphoramidites according to standard techniques, as per manufacturer's protocols (Glen Research, Sterling Va.).

(SEQ ID NO: 13)

G_{LNA}-G_{LNA}-(I)₁₅-G_{LNA}-T_{LNA}-C_{LNA}-G_{LNA}-T_{LNA}-T_{LNA}-
(I)₁₅-G_{LNA}-G_{LNA}

(SEQ ID NO: 14)

C_{LNA}-C_{LNA}-(C)₁₅-A_{LNA}-A_{LNA}-C_{LNA}-G_{LNA}-A_{LNA}-C_{LNA}-
(C)₁₅-C_{LNA}-C_{LNA}

(SEQ ID NO: 15)

G_{LNA}-G_{LNA}-(I)₁₅-G_{LNA}-T_{LNA}-C_{LNA}-G_{LNA}-T_{LNA}-T_{LNA}-

(SEQ ID NO: 16)

A_{LNA}-A_{LNA}-C_{LNA}-G_{LNA}-A_{LNA}-C_{LNA}-(C)₁₅-C_{LNA}-C_{LNA}

(SEQ ID NO: 17)

(I)₁₅-G_{LNA}-T_{LNA}-C_{LNA}-G_{LNA}-T_{LNA}-T_{LNA}

(SEQ ID NO: 18)

(C)₁₅-A_{LNA}-A_{LNA}-C_{LNA}-G_{LNA}-A_{LNA}-C_{LNA}

(SEQ ID NO: 19)

G_{LNA}-(I)₁₅-G_{LNA}-T_{LNA}-C_{LNA}-G_{LNA}-T_{LNA}-T_{LNA}-

(SEQ ID NO: 20)

C_{LNA}-(C)₁₅-A_{LNA}-A_{LNA}-C_{LNA}-G_{LNA}-A_{LNA}-C_{LNA}

(SEQ ID NO: 21)

C_{LNA}-G_{LNA}-(I)₁₅-G_{LNA}-T_{LNA}-C_{LNA}-G_{LNA}-T_{LNA}-T_{LNA}

(SEQ ID NO: 22)

G_{LNA}-C_{LNA}-(C)₁₅-A_{LNA}-A_{LNA}-C_{LNA}-G_{LNA}-A_{LNA}-C_{LNA}

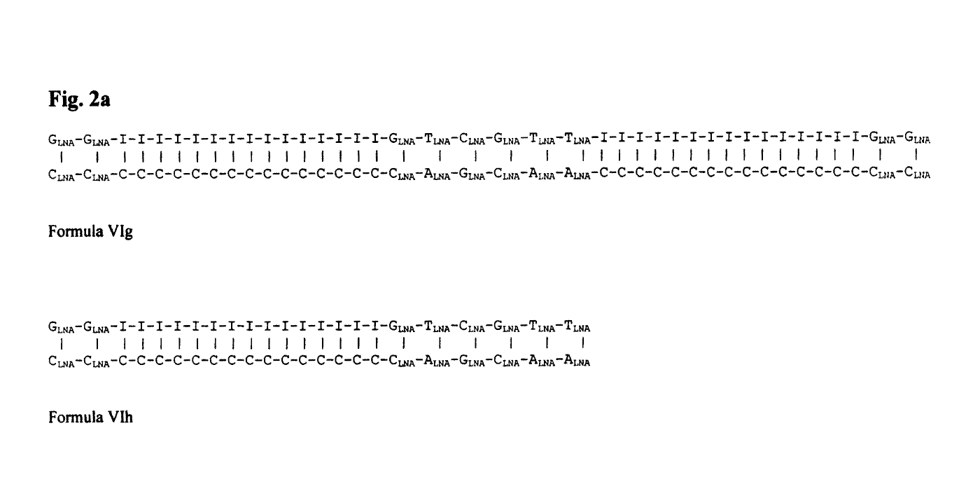

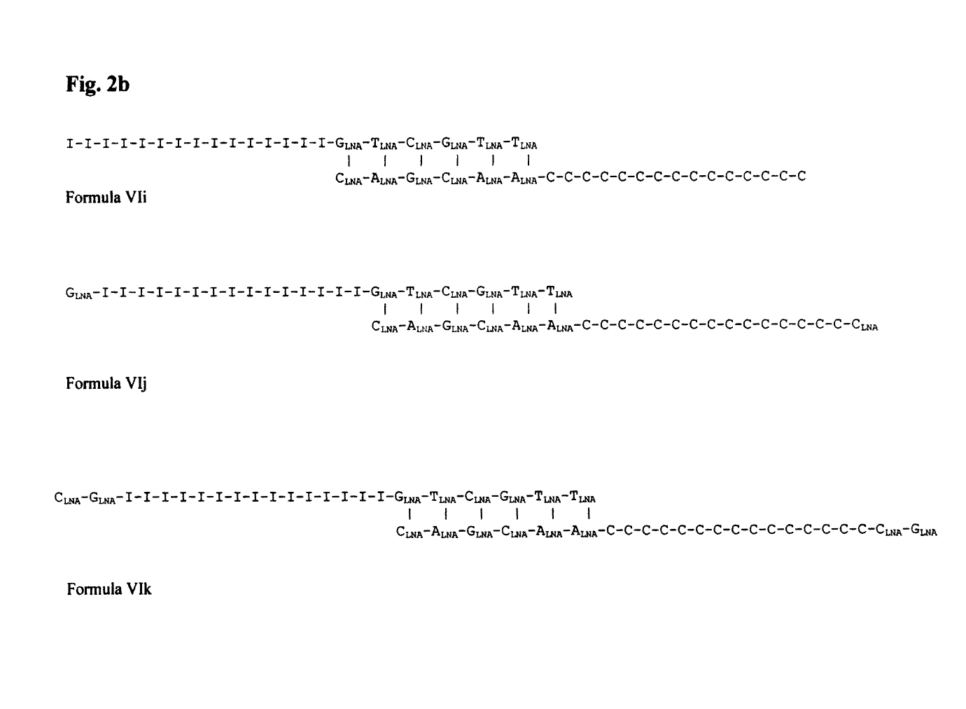

Equimolar amounts of each of SEQ ID NO: 13 and SEQ ID NO: 14, or SEQ ID NO: 15 and SEQ ID NO: 16, or SEQ ID NO: 17 and SEQ ID NO: 18, or SEQ ID NO: 19 and SEQ ID NO: 20, or SEQ ID NO: 21 and SEQ ID NO: 22 were combined and permitted to anneal to produce the dsRNA compound according to Formula VIg, VIh, VIi, VIj and VIk (FIG. 2).

### Example 7

### Poly IC/R Preparation

Molecular weights of poly I and poly C, and poly IC produced using the methods described were determined by SEC-MALLS (Tables 1 and 2).

Comparison of the SEC-MALLS data (Table 3) of SIGMA research grade (catalogue no. P4154, P4903 and P0913) and SAFC (Sigma-Aldrich Fine Chemicals) produced polymers indicates that the molecular weights of SAFC produced Poly I, Poly C, and Poly IC are greater than the SIGMA research grade material.

Small scale trials (16-20 mg scale) on the SAFC produced large MW Poly IC (MW 737,000) gave clear solution when complexed with Poly Arginine. When scaled up, the gradual addition of polyR took >8 hours and resulted in a hazy solution (table 4). Subsequent testing indicated that the hazy solution demonstrated TLR3/8 agonist activity similar to that of the smaller-scale production (data not shown).

Scaled-up trials (1-5 grams) on the SAFC-produced small MW Poly IC (MW 330,000) gave a clear solution when complexed with Poly Arginine (table 5). The addition time of Poly Arginine to Poly IC was less than half an hour and a clear solution was obtained after mixing the solution for less than an hour. The scale up of small MW Poly IC (4000.0 mg, MW 330,000) and Poly Arginine (2000 mg) gave a clear solution, and addition of poly Arginine to Poly IC was less than half an hour. A clear solution was obtained after mixing the solution for 1-2 hours.

Without wishing to be bound by theory, the length of the poly IC polymer may have an impact on preparation of the polyIC/R complex (time of mixing) without affecting TLR3/8 activity.

TABLE 1
Large Molecular Weight Poly I, Poly C, Poly IC SEC-MALLS Data

| Sample | Product | Lot Number | M_{W} |
|---|---|---|---|
| Poly I | W0893 | 18K4214 | 1,033,000 |
| Poly C | W0768 | 18K4213 | 745,000 |
| Poly IC | W0643 | 18K4228 | 737,000 |

TABLE 2
Small Molecular Weight Poly I, Poly C, Poly IC SEC-MALLS Data

| Sample | Product | Lot Number | M_{W} |
|---|---|---|---|
| Poly I | W0893 | 28K4208 | 291,500 |
| Poly C | W0768 | 28K4209 | 217,500 |
| Poly IC | W0643 | 28K4215 | 330,400 |

TABLE 3
Comparison of SEC-MALLS data for Sigma Research grade and
SAFC produced Poly I, Poly C and Poly IC

| Grade | Poly I | Poly C | Poly IC |
|---|---|---|---|
| Sigma | P4154, | P4903, | P0913, |
| Research | Lot # 107K4124 | Lot # 47K4149 | Lot # 57K4052 |
| | (Mw = 429,000) | (Mw = 125,000) | (Mw = 326,150) |
| SAFC | W 0893, | W 0768, | W0643, |
| Large MW | Lot # 18K4214 | Lot # 18K4213 | Lot # 18K4228 |
| polymers | (Mw = 1,033,000) | (Mw = 745,000) | (Mw = 737,000) |
| SAFC | W 0893, | W 0768, | W0643, |
| Small MW | Lot # 28K4208 | Lot # 28K4209 | Lot # 28K4215 |
| polymers | (Mw = 291,500) | (Mw = 217,500) | (Mw = 330,000) |

TABLE 4
Large Molecular Weight Poly (IC) W0643 (18K4228), and Poly Arginine W1018
(107K4220) weigh ups and concentrations (mg/ml)

| | Poly | water | | | | | Addition | Poly |
|---|---|---|---|---|---|---|---|---|
| | IC | added | Poly IC | Poly R | Buffer | polyR | Time | IC/R |
| Run # | (mg) | (ml) | (mg/ml) | (mg) | (ml) | (mg/ml) | (hr.) | mg/ml |
| 1 | 88.9 | 22.225 | 4.00 | 35.8 | 35.8 | 1.00 | 1-2 | 2.0 |
| | | | | | | | (clear) | |
| 2 | 4000.8 | 1000 | 4.00 | 2000.4 | 2000 | 1.00 | 6-8 | 2.0 |
| | | | | | | | (hazy)* | |

TABLE 5
Small Molecular Weight Poly (IC) W0643 (28K4215), and Poly Arginine W1018
(107K4220) weigh ups and concentrations (mg/ml)

| | | | | | | | Addition | |
|---|---|---|---|---|---|---|---|---|
| | Poly IC | Water | Poly IC | Poly R | Buffer | polyR | Time | Poly IC/R |
| Run # | (mg) | (ml) | (mg/ml) | (mg) | (ml) | (mg/ml) | (hr.) | mg/ml |
| 1 | 845.0 | 211.25 | 4.00 | 499.5 | 499.5 | 1.00 | 1-2 (clear) | 2.0 |
| 2 | 4003.5 | 1000.9 | 4.00 | 2000.5 | 2000.5 | 1.00 | 2-4 (clear) | 2.0 |

### Example 8

### Serum Stability of Adjuvant Compositions

HT29 cells express TLR-3 receptors, and will secrete Interfron Inducible Protein-10 (IP-10) when exposed to a TLR-3 agonist. The stimulatory activity (adjuvant activity) of a TLR-3 agonist may be measured by using this assay.

The amount of Poly IC/Poly R (Sigma), PolyICLC (Oncovir) and PolyIC (Sigma) were calculated and added to each tube to have final concentrations of 25, 12.5, 6.25, 3.13, 1.56, 0.78 and 0.4 ug/ml per well. Compositions comprising polyIC alone or in the presence of other adjuvants are incubated in 20% human AB serum (not heat-treated) in DMEM for 18-20 hours. The incubated serum samples comprising the polyIC and/or other adjuvants are added to HT-29 cells in culture and incubated for 24 hours. Following incubation, the supernatant is harvested and assayed for the presence and concentration of IP-10 (FIG. 3).

PolyL and polyR alone have no discernable TLR-3 agonist activity, nor does the medium+serum control. PolyIC pretreated with serum in the absence of the poly-L-lysine or polyarginine also lacks TLR-3 agonist activity beyond the negative control. Inclusion of poly-L-lysine alone or in the presence of carboxymethylcellulose, or polyarginine preserves the TLR-3 agonist activity of these adjuvant compositions.

Human serum comprises a range of nucleases, RNAses and proteases. Combining polyIC with poly-L-lysine alone or in the presence of carboxymethylcellulose, or polyarginine has a protective effect, in that the serum components have a reduced ability to degrade or otherwise inactivate the poly IC.

### Example 9

### Comparison of polyIC/L and PolyIC/R TLR-3 Agonist Activity

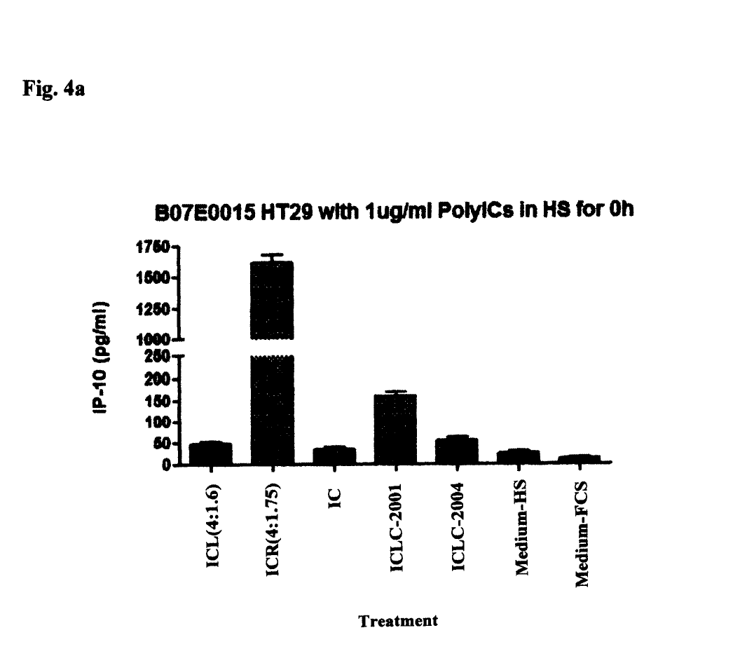

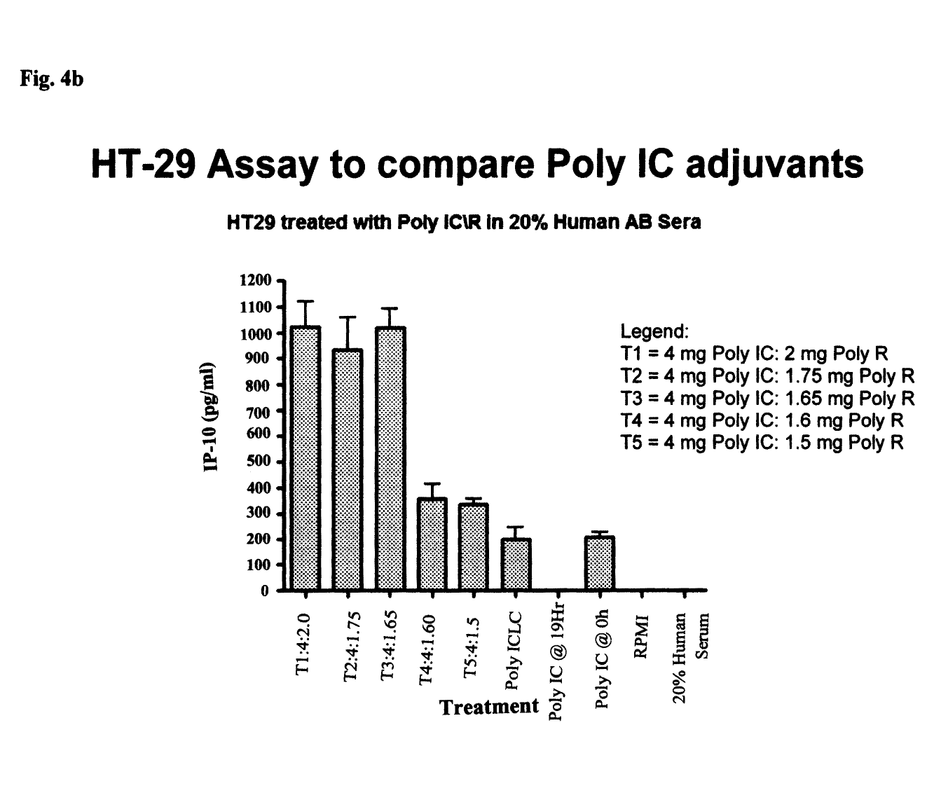

Serum pretreatment eliminated any TLR-3 agonist activity of the compositions comprising polyIC. In the absence of serum pretreatment, polyIC alone retains TLR-3 agonist activity (FIG. 4). Combining poly-L-lysine with polyIC before treating the HT-29 cells may provide a small increase in activity over polyIC alone. Combining polyarginine with polyIC before treating provides for a much more significant increase in TLR-3 agonist activity—almost 30-fold. Inclusion of carboxymethylcellulose (CMC) with the polyIC and poly-L-lysine also provides for an increase in TLR-3 agonist activity, but the effect is not as marked as with polyarginine. The difference in agonist activity observed between the two batches of polyIC+poly-L-lysine+CMC may be due to variation in the batch productions. The effect of concentration on the TLR-3 agonist activity is also apparent when comparing the data of FIGS. 3 and 4-FIG. 3 used 5 ug/ml polyIC for each of the treatments, while the data of FIG. 4 used 1 ug/ml polyIC.

FIG. 4b shows a specific comparison of the TLR-3 activity of 4 mg of polyIC in combination with 1.5-2 mg of polyarginine (a 4:1.5 to 4:2 mass ratio).

### Example 10

### Dendritic Cell Maturation

Dendritic cells (DC) are the most potent professional antigen-presenting cells. These cells play a role in activating T cells to initiate the adaptive immune response. For this study, dendritic cells were generated from CD 14+ monocytes cells and were differentiated into mature dendritic cells by various adjuvants. Immature dendritic cells were generated by treating CD14+ monocytes cells with 50 ng/ml of GMCSF and 50 ng/ml of IL-4 for 6 days. They were induced by various adjuvants for 48 hours to induce maturation of DC. Poly IC:LC, Poly IC, and Poly IC:R were each individually added to the immature DC to compare the level of DC maturation after 48 hours (Table 6). In contrast to the immature DC, Poly IC:R treated DC shows more DC maturation than Poly IC and Poly IC:LC. There were high expression levels of HLA-ABC, HLA-DR, and CD86 DC maturation marker in Poly IC:R. This shows that PolyIC:R is more potent compared to the other adjuvants in DC maturation and has a larger effect in the adaptive immune response.

TABLE 6
Dendritic cell maturation assay comparing polyIC adjuvants

| | Immature | IC | ICLC | IC/R |
|---|---|---|---|---|
| CD11c | 46 | 28 | 32 | 37 |
| MHC class I | 101 | 711 | 710 | 880 |
| MHC class II | 121 | 270 | 270 | 387 |
| CD86 | 315 | 720 | 720 | 893 |

The value in each cell represents the average intensity of fluorescent staining for the respective cell surface markers as assessed by FACS analysis. In immature DCs for CD86, the population had a cell surface intensity of 315, while on maturation with plyIC/R, the level of protein increased such that the cell surface intensity was increased t 893. PolyIC/R appears to be more potent than polyICLC for maturing dendritic cells.

### Example 11

### TLR-3 and TLR-8 Co-Stimulation

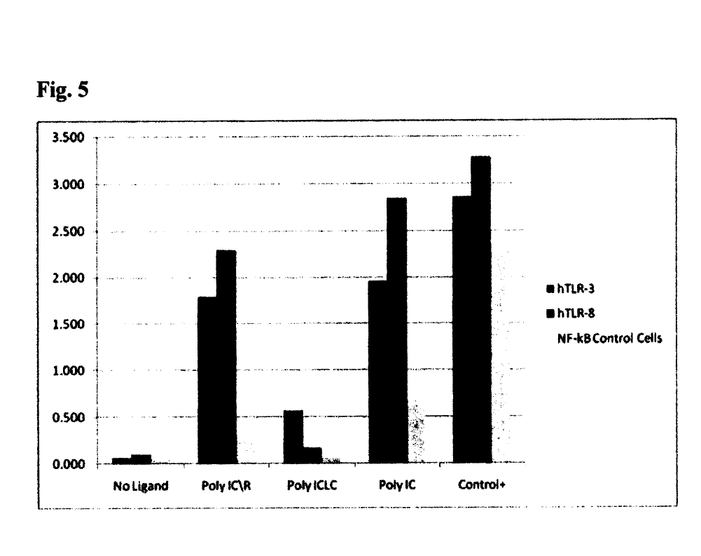

Poly IC\R and Poly IC demonstrate a similar agonist activity towards both TLR-3 and TLR-8. Poly ICLC demonstrates a more TLR-3 specific agonist activity, and with less potency than either of PolyIC/R or PolyIC. Human 293 cells are stably transfected with the various human TLR receptors. The agonist are then added and their ability to stimulate NF kappa B as a direct result of TLR receptor engagement induce the release of SEAP or secreted alkaline phosphatase (y asis). (FIG. 5).

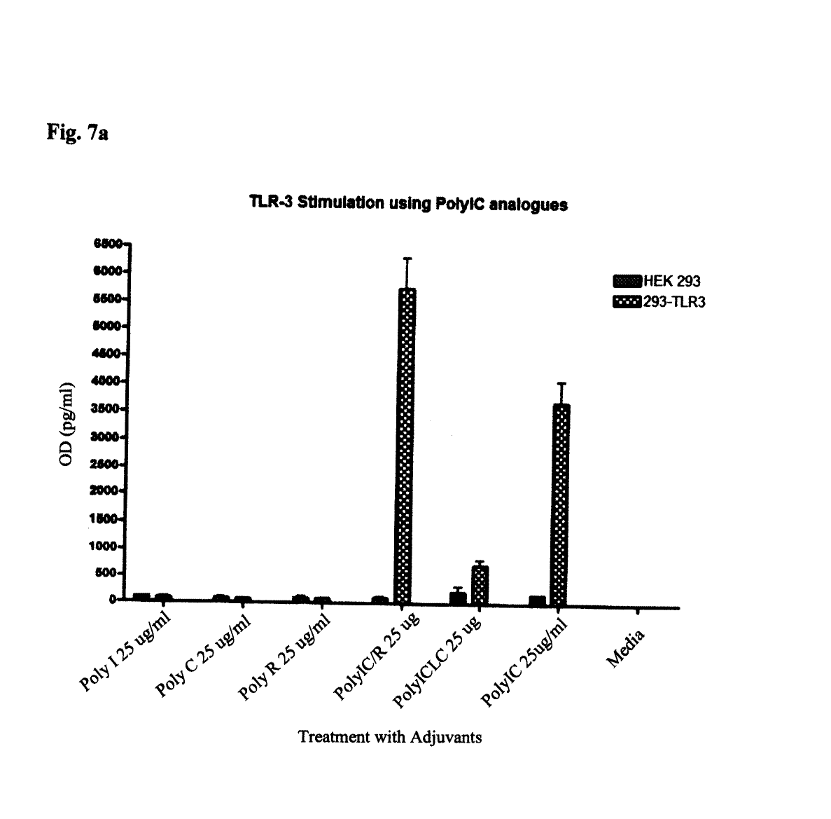

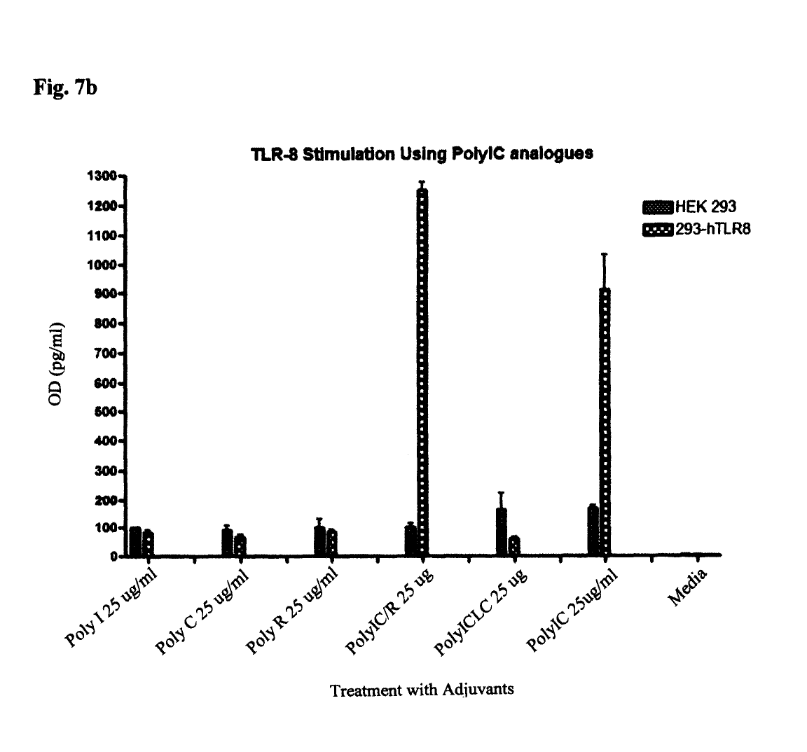

A second set of experiments investigated the individual components and their TLR3 and/or TLR8 agonist activity. (FIGS. 7A, B). TLR3 agonist activity (FIG. 7A) is observed predominantly in the polyIC/R, polyICLC and polyIC treatments, with the polyICLC demonstrating substantially less. Poly I, polyC (nucleotide homopolymers) or polyR alone demonstrate a slight agonist activity over the media negative control, but no difference in comparison to HEK293 cells not expressing TLR3.

PolyIC/R and poly IC both demonstrate TLR8 agonist activity (FIG. 7B), however polyICLC does not demonstrate significant agonist activity. The control HEK293 cells (not expressing TLR8) appear to respond to the agonist—this may be an artifact of the polyICLC preparation. TLR8 agonist activity of poly I, polyC or poly R alone does not differ significantly over the control HEK293 cells.

### Example 12

### Interferon Production in Human PBMC

Donor peripheral blood mononucleocytes (PBMC) were stimulated with a TLR-3 agonist and the secreted cytokines profiled. Interferon alpha, beta and gamma were quantified to investigate the activity of polyIC adjuvants.

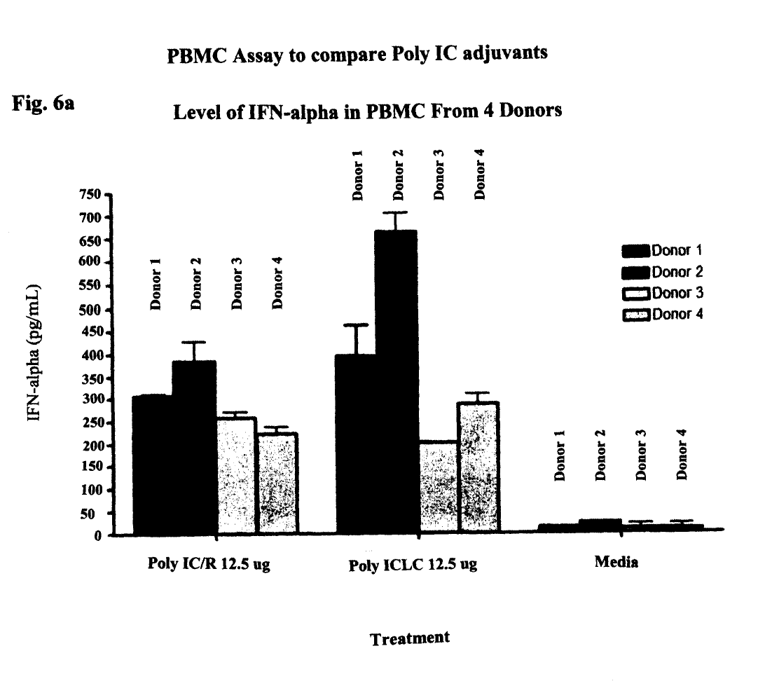

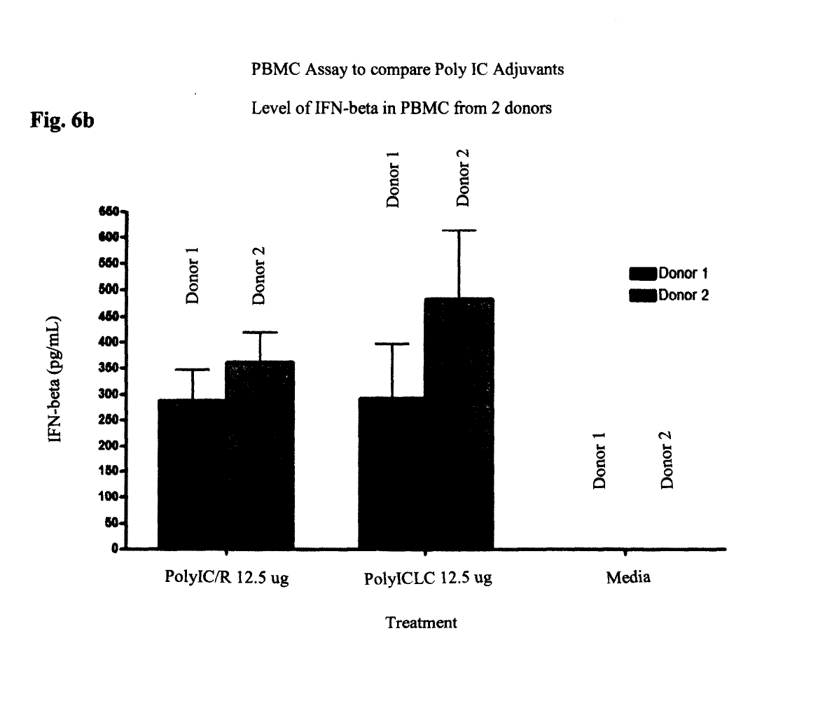

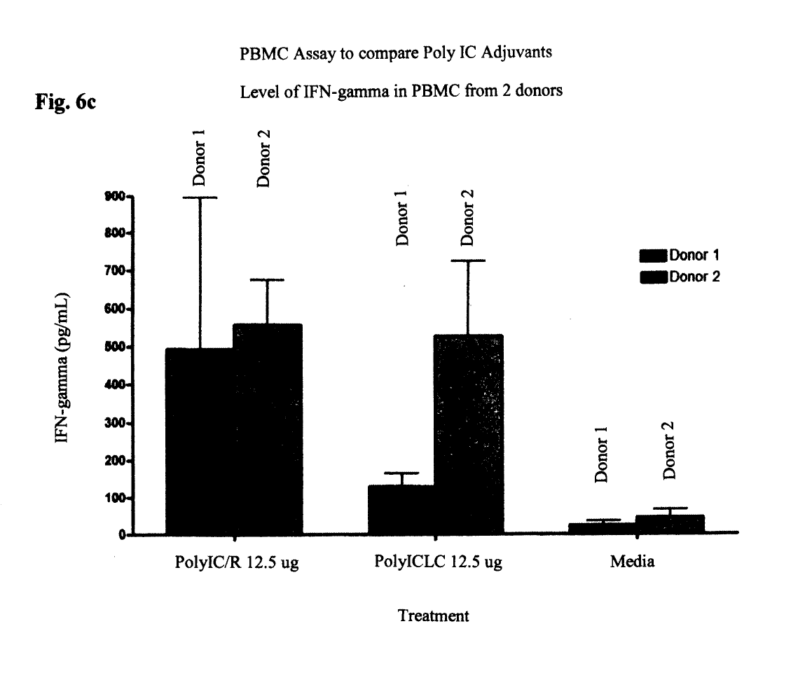

Results shown in FIGS. 6a, b and c indicate that both polyICLC and polyIC/R stimulate secretion of interferons, with some donor-donor variation.

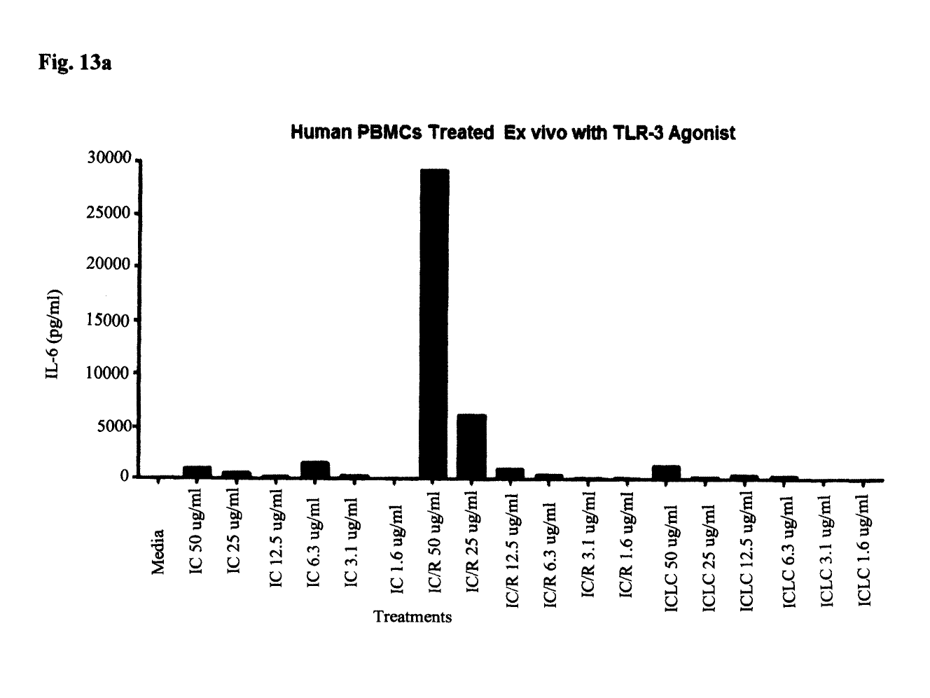

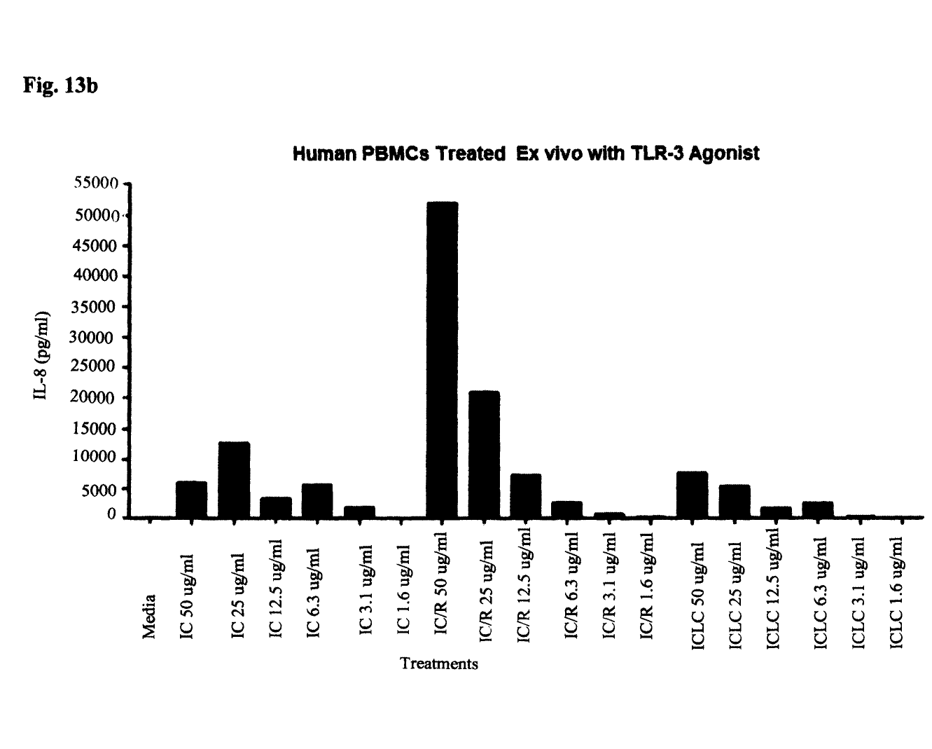

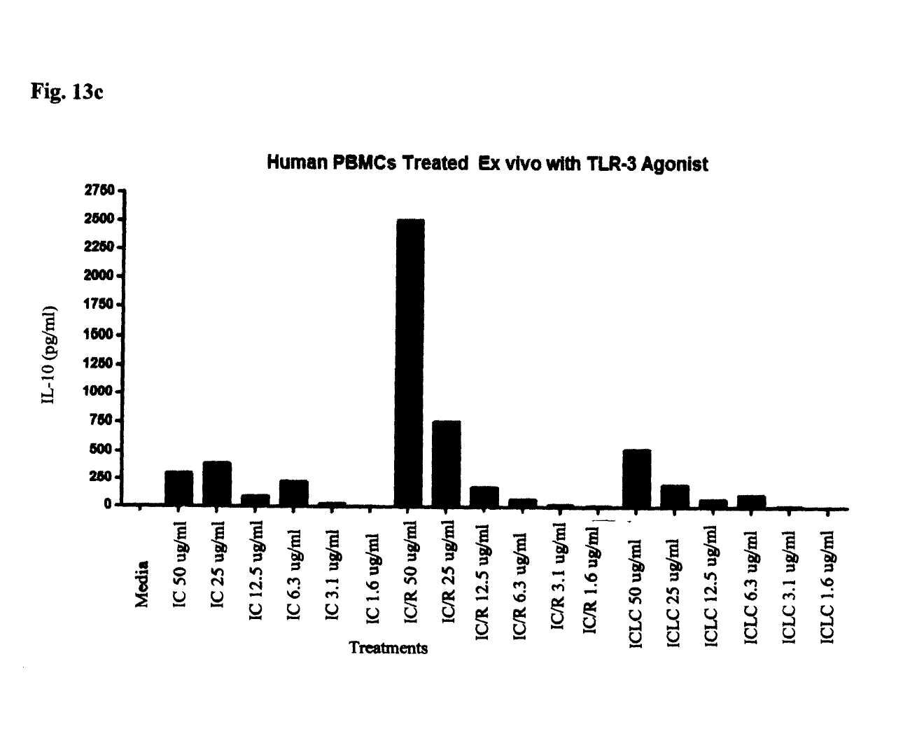

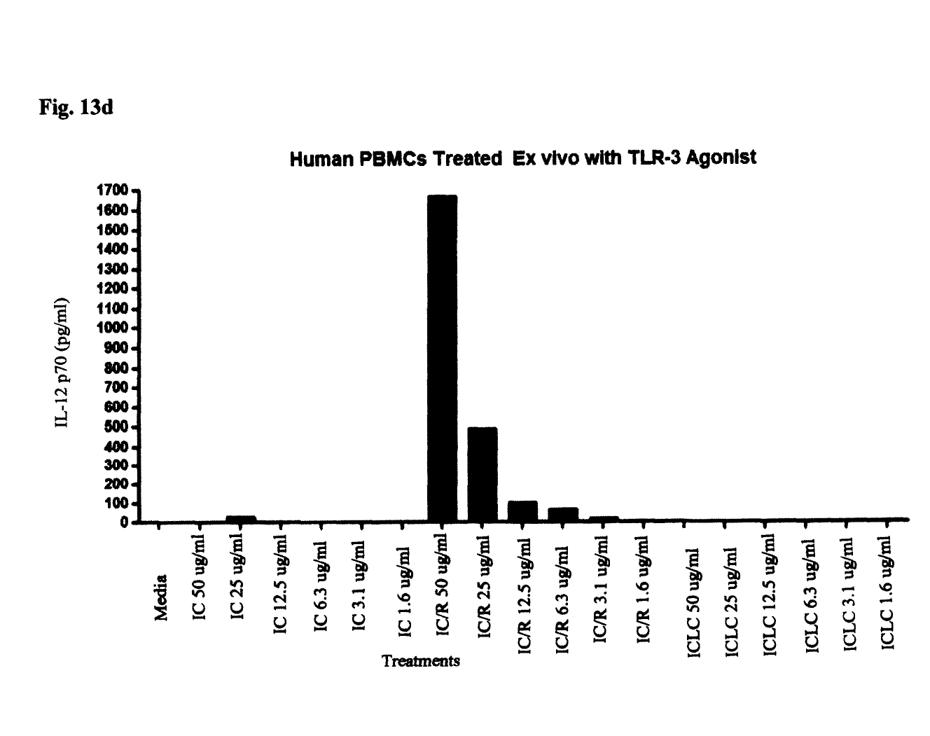

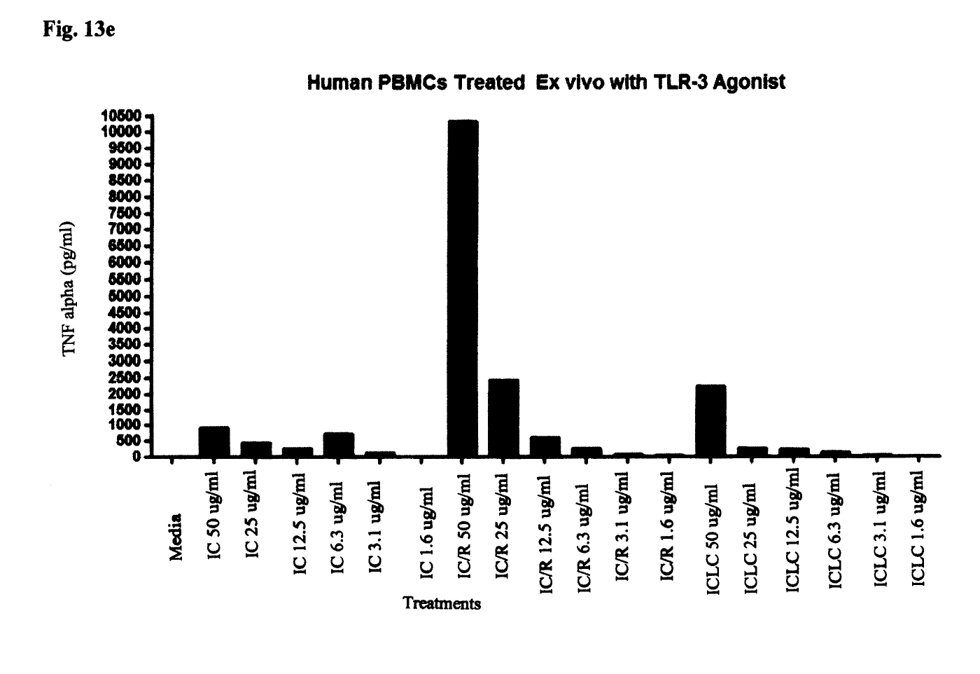

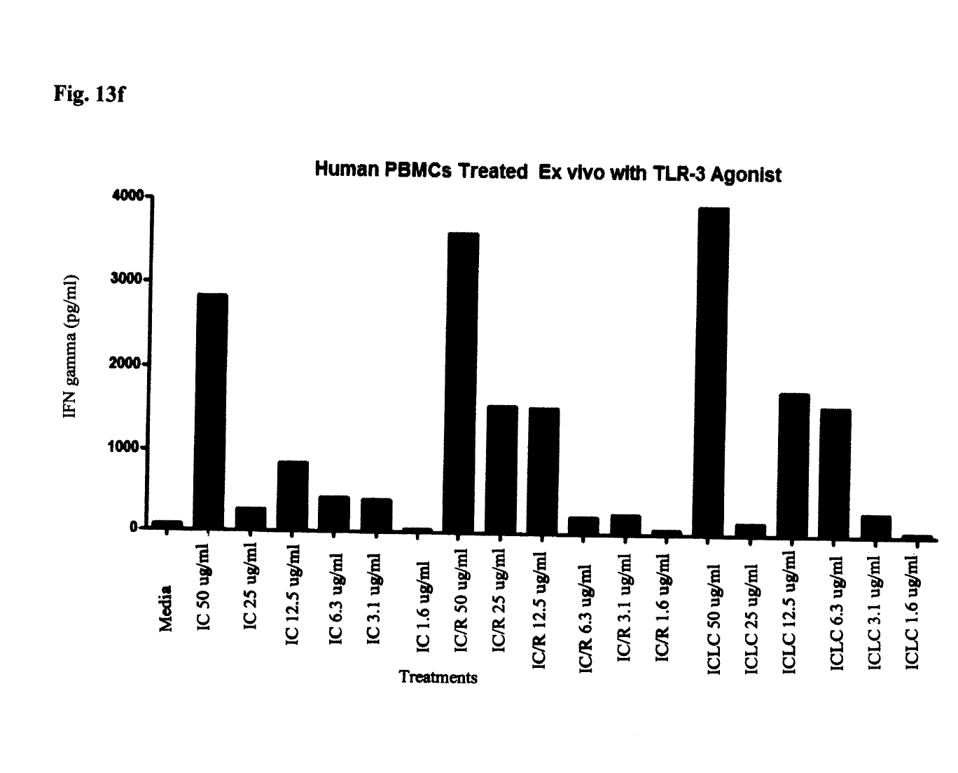

FIGS. 13 A-F show secreted cytokine levels of human PBMCs in response to polyIC, polyIC/R or IC/LC at concentrations ranging from 1.6 to 50 ug/ml. A-IL-6; B-IL-8; C-IL-10; D-IL-12 p70; E-TNF alpha; F-IFN gamma. Poly IC/R stimulates production of all six cytokines in human PBMCs, and to a greater level compared to polyIC or polyIC/LC. The stimulation of human PBMCs by polyIC/R is also dose-dependent, as illustrated by the titerable effect observed. For some cytokines, superior agonist activity of polyIC/R was observed with as little as 6.3 ug/ml (IL-12).

### Example 13

### Rnase A hydrolysis of PolyIC/Lysine and PolyIC/Arginine

The Rnase stability of the polyIC in the presence or absence of a polycationic polymer was investigated. Samples of polyIC alone, or in combination with a polycationic polymer was incubated at 37° C. for 90 minutes, with a sample taken periodically and the spectrophotometric absorbance at 260 nm determined. PolyIC alone, or combined with lysine (4:2 mg/ml and 4:1.6 mg/ml mass:mass ratio) or arginine (4:1.5 and 4:2 mg/ml mass:mass ratio) were compared, as was a preparation of polyICLC (obtained from Oncovir).

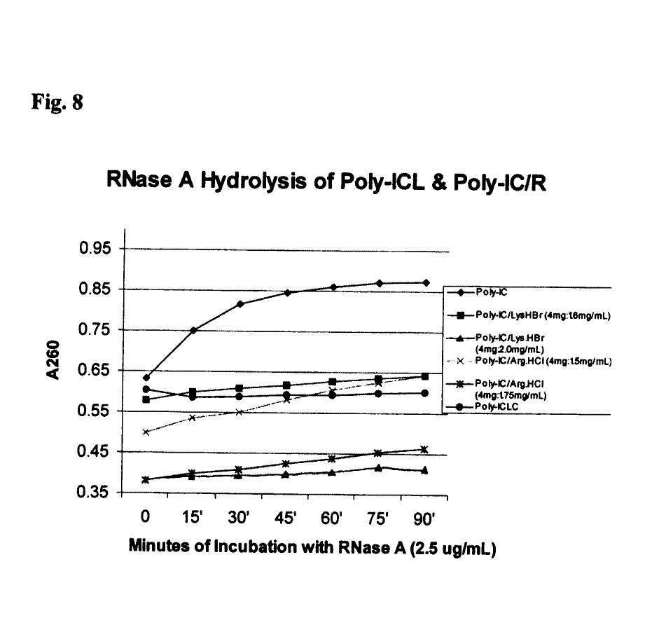

FIG. 8 shows the changes in A₂₆₀ over the 90 minute incubation. PolyIC, in the absence of a polycationic polymer is degraded by the Rnase A, while the inclusion of polyarginine or polylysine inhibited this degradation. The 4:1.5 mg/ml polyIC/arginine reduced the degradation significantly compared to the untreated polyIC, but some degradation had occurred by the time of the first sample.

Both lysine and arginine in a 4:2 mass:mass ratio with polyIC offered significantly greater protection from Rnase degradation

### Example 14

### IP-10 Level in HT-29 Cells—Effect of Varying Order of Addition of Agonist Components

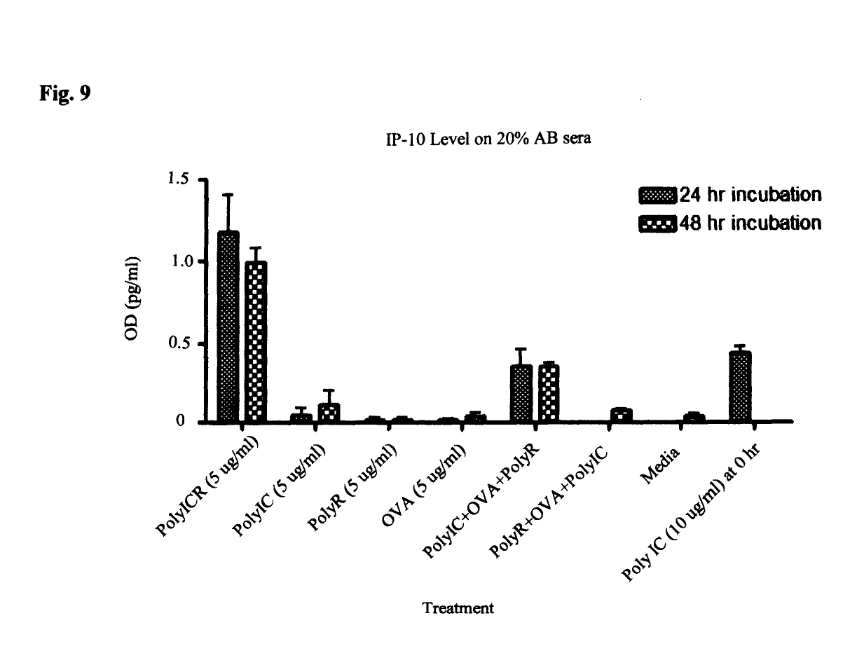

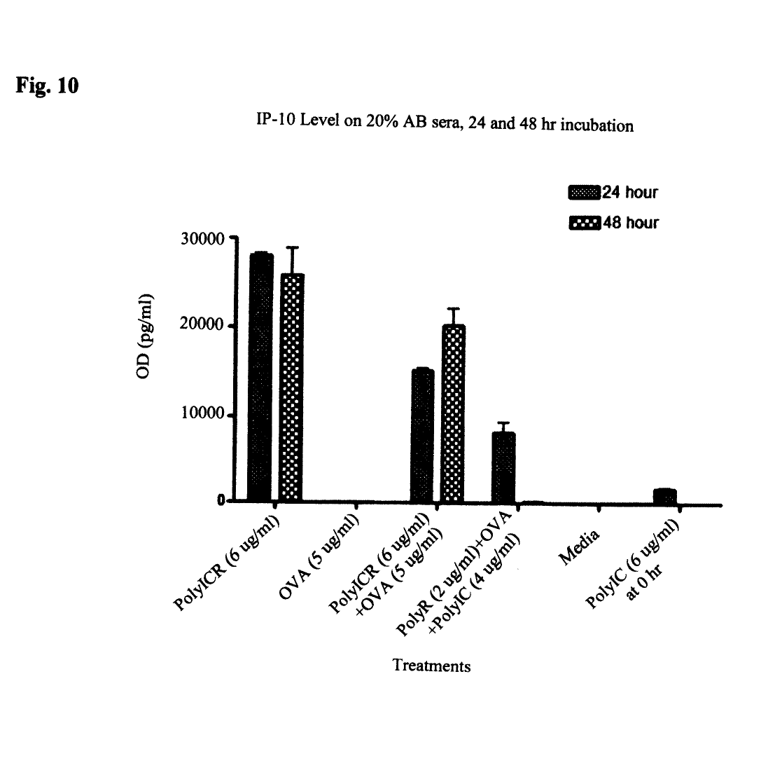

To demonstrate the effectiveness of varying the sequence of addition of components (polyIC, OVA and/or polyR) of an agonist composition, quantitative measurement of human IP-10 was performed as described as an indicator of TLR-3 agonist activity. The amount of Poly R (Sigma P7762), Po1yICR (Nventa), PolyIC (Sigma P9582) and Ovalbumin (OVA; Sigma A7641)) were calculated and added to each tube to have final concentrations of 5-10 ug/ml as indicated in FIGS. 9 and 10. The different combinations of PolyIC,poly R and OVA tested, and their relative order of addition are indicated in Tables 7 and 8. Compositions were mixed following each addition (e.g. first and second components were added and mixed, followed by addition of a third component, and additional mixing). Tubes were incubated overnight at room temperature in 20% human AB serum, and assayed as described.

TABLE 7
Order of addition of agonist compositions for FIG. 9

| Composition (sample designation) | first | second | third |
|---|---|---|---|
| polyICR (5 ug/ml) + OVA | polyIC | polyR | OVA |
| Poly IC (5 ug/ml) + OVA | polyIC | OVA | |
| PolyR (5 ug/ml) + OVA | polyR | OVA | |
| OVA (5 ug/ml) | OVA | | |
| PolyIC + OVA + polyR | polyIC | OVA | polyR |
| PolyR + OVA + polyIC | polyR | OVA | POlyIC |
| media | media | | |
| polyIC (10 ug/ml) at 0 hr | polyIC | | |

TABLE 8
Order of addition of agonist compositions for FIG. 10

| Composition (sample designation) | first | second | third |
|---|---|---|---|
| polyICR (6 ug/ml) | polyIC | polyR | |
| OVA (5 ug/ml) | OVA | | |
| polyICR (6 ug/ml) + OVA (5 ug/ml) | polyIC | polyR | OVA |
| PolyR (2 ug/ml) + OVA + polyIC (4 ug/ml) | polyR | OVA | PolyIC |
| Media | media | | |
| PolyIC (6 ug/ml) at 0 hr | poly IC | | |

FIGS. 9 and 10 show the varying levels of IP-10 produced in response to the agonist compositions, illustrating a stimulatory effect of compositions comprising polyIC, further enhanced by the inclusion of polyR. OVA, media, polyR or polyIC alone demonstrated little to no TLR-3 agonist activity. Compositions comprising polyR, polyIC and OVA varied in their TLR-3 agonist activity according to the order in which the components were combined. Premixing polyIC with polyarginine (polyIC and polyR combined first and second, followed by addition of the antigen-leftmost sample in both FIGS. 9 and 10) demonstrated the highest level of TLR-3 agonist activity. Combining polyR first with OVA, followed by addition of polyIC yielded a composition that demonstrated some TLR-3 agonist activity at 24 hours, but this decreased to background by 48 hours.

### Example 15

### IFN-Gamma Production in Stimulated Splenocytes from Mice Immunized with HspE7 and PolyIC or ICR at 100 ug (ELISpot)

To determine if co-immunization of HspE7 with a TLR3 agonist will augment the ability of HspE7 to induce E7-specific cellular immunity, mice were immunized with PBS, HspE7 alone, or 500 ug of HspE7 with 100 ug of either polyICR or polyIC. Harvested splenocytes were restimulated with media, an unrelated antigen (influenza NP) or E7 peptide, and ELISpot was performed as described.

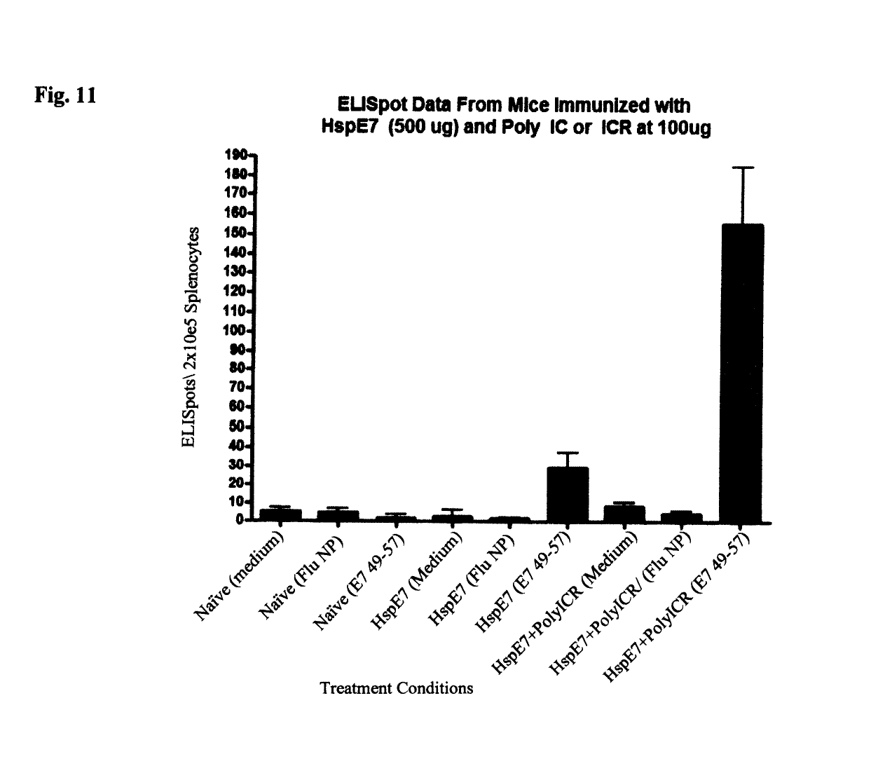

Results are illustrated in FIG. 11. Splenocytes from naïve mice (PBS-control immunization) did not demonstrate significant IFN-gamma production following restimulation. Splenocytes from mice immunized with HspE7 alone exhibited a low level of IFN-gamma production following restimulation with E7 peptide. Splenocytes from mice immunized with HspE7 with polyICR did not demonstrate significant IFN-gamma production following restimulation with media alone or an unrelated antigen (influenza NP), however a pronounced and significant increase in IFN-gamma production was observed following restimulation with a specific antigen (E7 peptide).

### Example 16

### Efficacy of PolyIC/R for Tumor Regression in TC-1K Tumors in C57BL/6 Mice

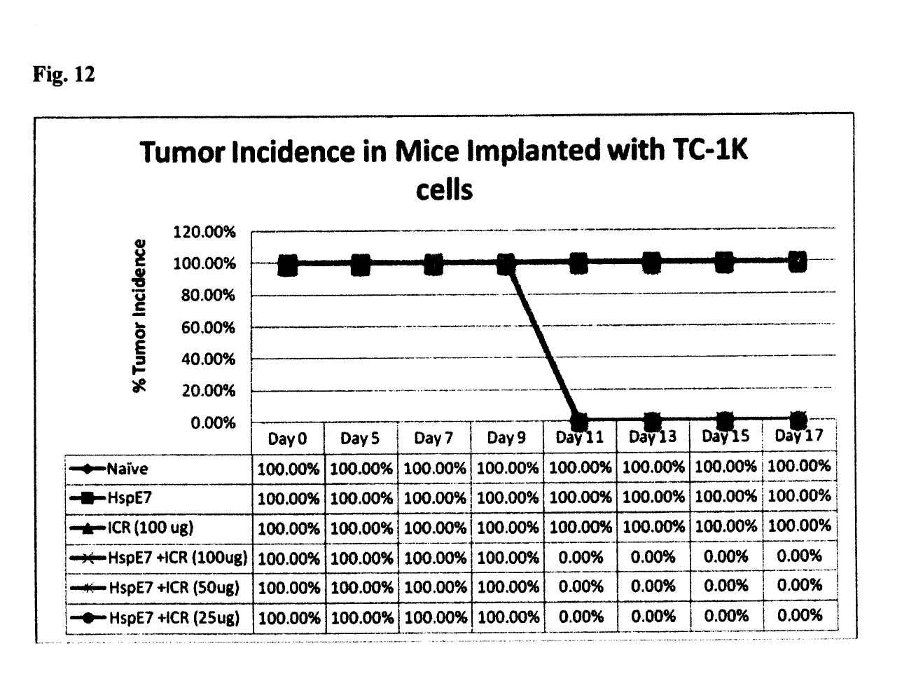

This study was conducted to evaluate tumor incidence in C57BL/6 mice after subcutaneous implantation of tumor cells (5 mice per group). Results are illustrated in FIGS. 12 and 13, and their accompanying tables. For TC-1K tumors (FIG. 12), naïve, HspE7 and ICR (100 ug) treated mice demonstrated 100% tumor incidence from day 0 to day 17. Mice treated with the immunogen+ICR composition (HspE7+ICR (100 ug), HspE7+ICR (50 ug) and HspE7+ICR (25 ug)) all demonstrated 100% tumor incidence from day 0 to day 9, which decreased to 0% from day 11 onward.

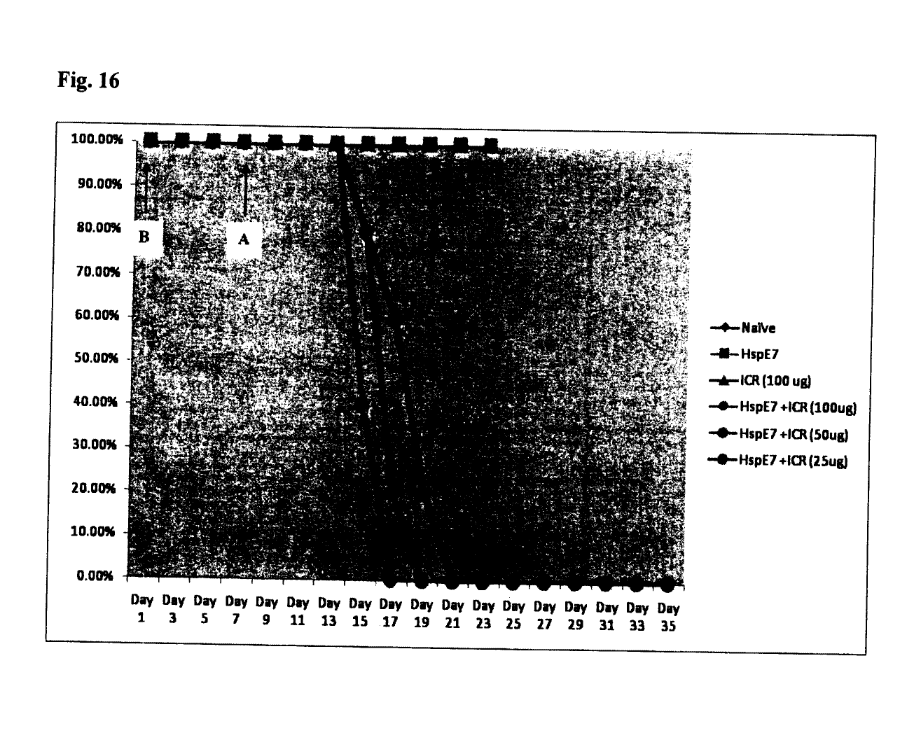

FIG. 16 shows the results of tumor regression studies in the same manner as described, with data points to day 35 (5 mice per group). Tumor cells were implated on day 1, and the treatments started on day 7. For TC-1K tumors, naïve, HspE7 and ICR (100 ug) groups, 100% tumor incidence was observed out to day 23, at which point the mice were sacrificed. Tumor regression was observed starting on day 15 in mice treated with HspE7+ICR at 25, 50 or 100 ug. Tumors continued to shrink over the next week week, with all mice demonstrating reduced tumor mass (to zero or thereabouts) by day 21.

Without wishing to be bound by theory, immunization with an antigen (e.g. HspE7) in combination with polyIC/R induces a CD8+ response sufficient to reduce or eliminate established tumors in two murine models. Further growth and/or spreading of tumors in the mice was prevented in both mouse models.

### Example 17

### Efficacy of Poly IC/R for Prevention of Establishment of E7 Antigen-Positive TC-1K Tumors

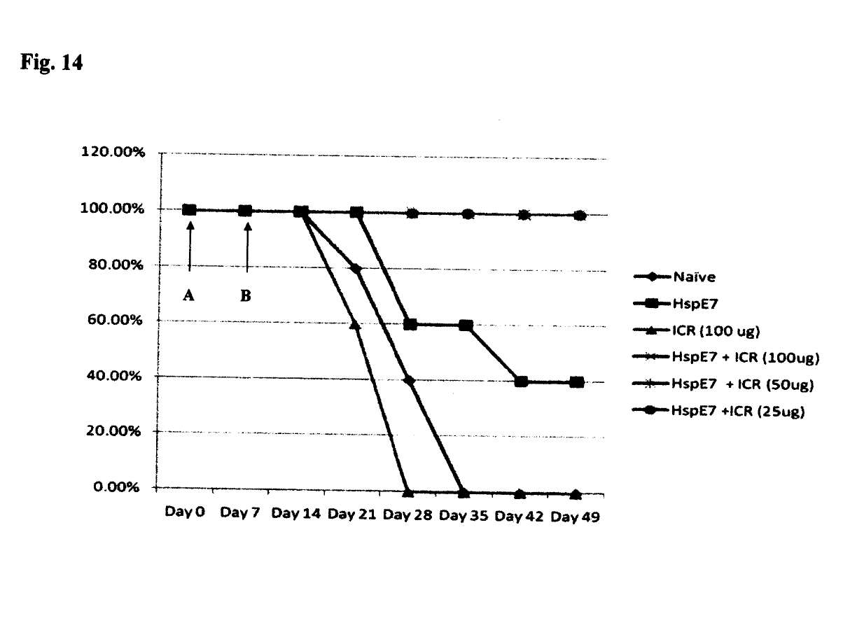

Mice (5 mice per group) were administered the HspE7, IC/R or combinations of HspE7+IC/R, followed by subcutaneous implantation of TC-1K tumor cells; results are shown in FIG. 14. Tumor formation was first observed in naïve, HspE7 or ICR—treated mice at day 14, with tumor growth continuing, or new tumors establishing over time. Tumors were observed in the naïve and IC/R treated animals starting on day 21, and in the HspE7 treated animals starting on day 28. Animals treated with HspE7+IC/R remained tumor free for the duration of the study.

Without wishing to be bound by theory, immunization with an antigen (e.g. HspE7) in combination with polyIC/R in advance of tumor cell implantation induces an immune response sufficient to prevent establishment of tumors in a murine tumor model.

### Example 18

### Effect of Alum in Combination with IC/R on Antigen Specific Antibody Response

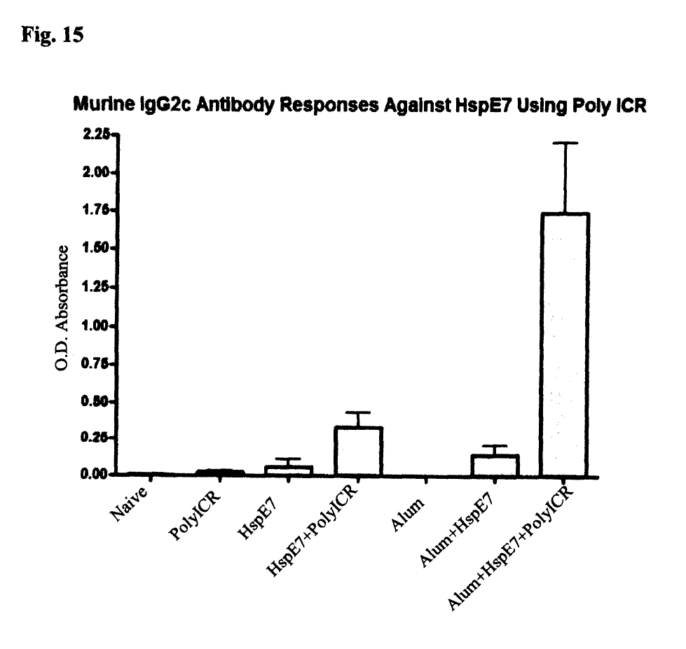

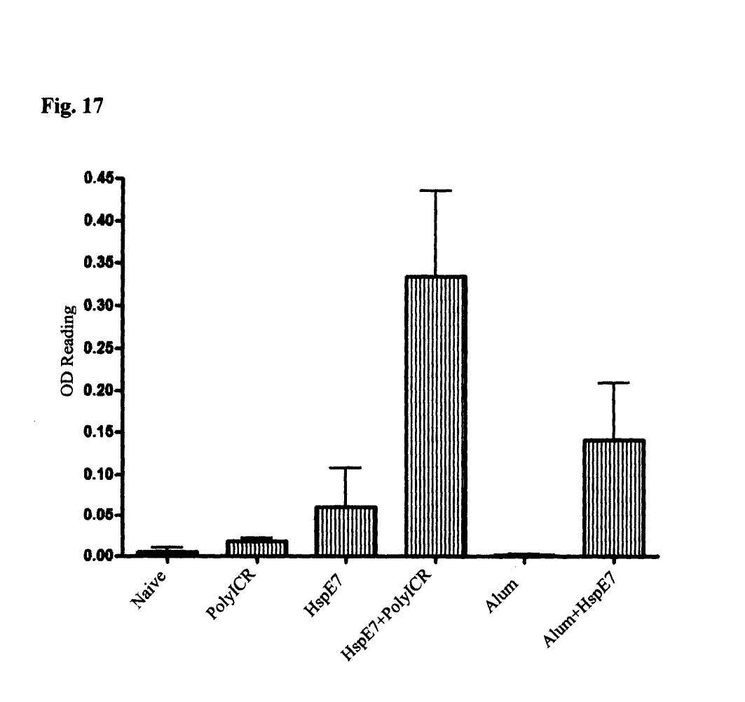

Mice were administered poly IC/R, HspE7 (400 ug) antigen alone or poly IC/R (100 ug)+antigen (HspE7) with or without alum (100 ul alum per dose). Two doses at day 0 and 21 were administered, and serum drawn on day 42. Serum IgG2c antibody response to HspE7 was measured (FIGS. 15, 17). Mice that did not receive antigen (naïve, polyICR or alum treatments) exhibited effectively background antigen-specific antibody production, although the polyICR treated mice had a slight response. HspE7 alone or in combination with alum yielded some level of antigen-specific antibody production, and inclusion of polyICR in combination with the HspE7 antigen increased the antigen-specific response. A surprising increase in the antigen-specific antibody response was observed in mice administered the HspE7+polyICR in combination with alum—approximately a 3-fold increase over the additive effect of ICR and alum individually.

FIG. 17 shows the data of FIG. 15, with the data for the HspE7+PolyIC/R+Alum omitted. A comparison of the antigen-specific stimulation of the two adjuvants individually (Alum and polyIC/R) clearly illustrates the superior immunostimulatory effect of poly IC/R (approximately twice that of alum).

Poly ICR demonstrates significant adjuvant activity and enhances the antigen-specific antibody response in a subject to whom it is administered. A further, synergistic adjuvant activity is observed when polyICR is combined with alum.

All citations are herein incorporated by reference.

One or more currently preferred embodiments have been described by way of example. It will be apparent to persons skilled in the art that a number of variations and modifications can be made without departing from the scope of the invention as defined in the claims.

What is claimed is:

1. An adjuvant composition comprising:
polyIC and a cationic polymer;
the cationic polymer comprising from about 100 to about 700 amino acid residues;
the mass:mass ratio of the polyIC:cationic polymer is from about 4:1.5 to about 4:3.

2. The composition of claim 1, wherein the polyIC has a average molecular mass from about 100 to about 5000 kDa.

3. The composition of claim 1, further comprising an immunogen.

4. The composition of claim 3 wherein the immunogen is present at an amount from about 0.1 ug to about 20 mg.

5. The composition of claim 3, wherein the immunogen comprises a bacterial, viral, tumor-derived or fungal immunogen.

6. The composition of claim 1 wherein the cationic polymer is polyarginine, polyornithine or polylysine.

7. The composition of claim 5 wherein the immunogen is purified HspE7.

8. The composition of claim 1, further comprising a second adjuvant composition.

9. The composition of claim 8, wherein the second adjuvant composition comprises carboxymethylcellulose, poly-L-lysine, aluminium hydroxide, alum, aluminum trihydrate or other aluminium-comprising salts, virosomes, nucleic acids comprising CpG motifs, squalene, oils, saponins, virus-like particles, monophosphoryl-lipidA/trehalose dicorynomycolate, toll-like receptor agonists or copolymers such as polyoxypropylene and polyoxyethylene.

10. A method of inducing dendritic cell maturation comprising:
administering to a cell a composition comprising polyIC and a cationic polymer;
the cationic polymer comprising from about 100 to about 700 amino acid residues;
the mass:mass ratio of the polyIC:cationic polymer is from about 4:1.5 to about 4:3.

11. The method of claim 10, wherein the polyIC has an average molecular mass from about 100 to about 5000 kDa.

12. The method of claim 11, the composition further comprising an immunogen.

13. The method of claim 12 wherein the immunogen is present at an amount from about 0.1 ug to about 20 mg.

14. The method of claim 10 wherein the cationic polymer is polyarginine, polyornithine or polylysine.

15. The method of claim 12, wherein the immunogen comprises a bacterial, viral, tumor-derived or fungal immunogen.

16. The method of claim 14 wherein the immunogen is purified HspE7.

17. The method of claim 10, wherein the composition further comprises a second adjuvant composition.

18. The composition of claim 17, wherein the second adjuvant composition comprises carboxymethylcellulose, poly-L-lysine, aluminium hydroxide, alum, aluminum trihydrate, or aluminium-comprising salts, virosomes, nucleic acids comprising CpG motifs, squalene, oils, saponins, virus-like particles, monophosphoryl-lipidA/trehalose dicorynomycolate, toll-like receptor agonists or copolymers such as polyoxypropylene and polyoxyethylene.

19. A TLR8 agonist comprising:
polyIC and a cationic polymer;
the cationic polymer comprising from about 100 to about 700 amino acid residues, and
the mass:mass ratio of the polyIC:cationic polymer is from about 4:1.5 to about 4:3.

20. A method of stabilizing a polyIC complex comprising combining polyIC with a cationic polymer comprising from about 100 to about 700 amino acid residues, to obtain a mass:mass ratio of the polyIC:cationic polymer of from about 4:1.5 to about 4:3.

21. A method of making an adjuvant composition comprising:
combining a solution of polyIC and a solution of a cationic polymer, in a mass:mass ratio of the polyIC:cationic polymer from about 4:1.5 to about 4:3; and wherein the cationic polymer comprising from about 100 to about 700 amino acid residues.

22. A method of decreasing the size or number, or size and number, of tumors or tumor cells in a subject, the method comprising:
administering to a subject a composition comprising polyIC and a cationic polymer;
the cationic polymer comprising from about 100 to about 700 amino acid residues;
the mass:mass ratio of the polyIC:cationic polymer is from about 4:1.5 to about 4:3.

23. The method of claim 22 wherein the composition further comprises an immunogen found in the tumor or tumor cells.

24. A method of inducing an immune cell comprising:
administering to a cell, a composition comprising polyIC and a cationic polymer;
the cationic polymer comprising from about 100 to about 700 amino acid residues;
the mass:mass ratio of the polyIC:cationic polymer is from about 4:1.5 to about 4:3.

25. A method of inducing cytokine production in a cell comprising:
administering to a cell a composition comprising polyIC and a cationic polymer;
the cationic polymer comprising from about 100 to about 700 amino acid residues;
the mass:mass ratio of the polyIC:cationic polymer is from about 4:1.5 to about 4:3.

26. The method of claim 25, wherein the cell is a peripheral blood mononuclear cell.

27. The method of claim 25, wherein the cytokine is selected from the group consisting of IL-1α, IL-1β, IL-2, 11-4, IL-5, IL-6, IL-7, IL-8, IL-9, IL-10, IL-12 (p70), IL-13, IL-15, IL-17, IL-18, IFNα (alpha), IFNβ (beta), IFNγ (gamma), GM-CSF, TNFα (alpha), G-CSF, MIP-1α (alpha), MIP-1β (beta), MCP-1, EOTAXIN, RANTES, FGF-basic and VEGF.

28. The method of claim 24, wherein the immune cell is a peripheral blood mononuclear cell, a dendritic cell or a CD8+ cell.

29. A method of inducing an immune response to an immunogen in a subject, comprising:
administering to a subject a composition comprising an immunogen, polyIC and a cationic polymer;
the cationic polymer comprising from about 100 to about 700 amino acid residues;
the mass:mass ratio of the polyIC:cationic polymer is from about 4:1.5 to about 4:3;
the immune response comprising one or more than one of a decrease in size, number or size and number of tumors in the subject, induction of an immune cell, induction of dendritic cell activation or induction of cytokine production.

30. A method of inducing an immunogen-specific antibody response in a subject, comprising:
administering to a subject a composition comprising polyIC, a cationic polymer, and immunogen;
the cationic polymer comprising from about 100 to about 700 amino acid residues;
the mass:mass ratio of the polyIC:cationic polymer is from about 4:1.5 to about 4:3.

31. The method of claim 29, wherein the composition further comprises a second adjuvant composition.

32. The method of claim 30, wherein the second adjuvant composition comprises carboxymethylcellulose, poly-L-lysine, aluminium hydroxide, alum, aluminum trihydrate or aluminium-comprising salts, virosomes, nucleic acids comprising CpG motifs, squalene, oils, saponins, virus-like particles, monophosphoryl-lipidA/trehalose dicorynomycolate, toll-like receptor agonists or copolymers such as polyoxypropylene and polyoxyethylene.

33. An adjuvant composition for topical administration, comprising:
polyIC and a cationic polymer, and a topical base;
the cationic polymer comprising from about 100 to about 700 amino acid residues;
the mass:mass ratio of the polyIC:cationic polymer is from about 4:1.5 to about 4:3.

34. A method of inducing a localized immune response at or near an epithelial surface comprising:
applying a composition comprising PolyIC and a cationic polymer, and a topical base to an epithelial surface of a subject;
the cationic polymer comprising from about 100 to about 700 amino acid residues;
the mass:mass ratio of the polyIC:cationic polymer is from about 4:1.5 to about 4:3.
